SEQUENCE LISTING (28 sequences)

SEQ ID NO: 1 - DNA, 50 bp, Mycobacteriophage Bxb1
  source 1..50: mol_type = genomic DNA
tcggccggcttgtcgacgacggcggtctccgtcgtcaggatcatccgggc

SEQ ID NO: 2 - DNA, 63 bp, Mycobacteriophage Bxb1
  source 1..63: mol_type = genomic DNA
gtcgtggtttgtctggtcaaccaccgcggtctcagtggtgtacggtacaaaccccgacggtaa

SEQ ID NO: 3 - DNA, 102 bp, synthetic construct
  misc_feature 1..102: note = Furin-T2A linker(pDeliver)
  source 1..102: mol_type = other DNA
ggcatcaggaggaagaggagcgtgagccacggaagcggaggaagcggagagggcaggggaagtcttctaacatgcggggacgtggaggaaaatcccggcccc

SEQ ID NO: 4 - DNA, 108 bp, synthetic construct
  misc_feature 1..108: note = Npu DnaE-C
  source 1..108: mol_type = other DNA
atgatcaagatcgccaccaggaagtacctgggcaagcagaacgtgtacgacatcggcgtggagagggaccacaacttcgccctgaagaacggcttcatcgccagcaac

SEQ ID NO: 5 - DNA, 306 bp, synthetic construct
  misc_feature 1..306: note = Npu DnaE-N
  source 1..306: mol_type = other DNA
tgcctgagctacgagaccgagatcctgaccgtggagtacggcctgctgcccatcggcaagatcgtggagaagaggatcgagtgcaccgtgtacagcgtggacaacaacggcaacatctacacccagcccgtggcccagtggcacgacaggggcgagcaggaggtgttcgagtactgcctggaggacggcagcctgatcagggccaccaaggaccacaagttcatgaccgtggacggccagatgctgcccatcgacgagatcttcgagagggagctggacctgatgagggtggacaacctgcccaac

SEQ ID NO: 6 - DNA, 117 bp, synthetic construct
  misc_feature 1..117: note = C-terminal BsrR
  source 1..117: mol_type = other DNA
tgtagggagttgatttcagactatgcaccagattgttttgtgttaatagaaatgaatggcaagttagtcaaaactacgattgaagaactcattccactcaaatatacccgaaattaa

SEQ ID NO: 7 - DNA, 306 bp, synthetic construct
  misc_feature 1..306: note = N-terminal BsrR
  source 1..306: mol_type = other DNA
atgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatg

SEQ ID NO: 8 - DNA, 587 bp, synthetic construct
  misc_feature 1..587: note = Internal ribosome entry site (IRES)
  source 1..587: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 9 - DNA, 720 bp, synthetic construct
  misc_feature 1..720: note = EGFP
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 10 - DNA, 386 bp, synthetic construct
  misc_feature 1..386: note = pPlatform landing pad - (BXB1 attB - Furin-T2A - Npu DnaE - BsrR(C-term)
  source 1..386: mol_type = other DNA
tgataatagtcggccggcttgtcgacgacggcggtctccgtcgtcaggatcatccgggcggcatcaggaggaagaggagcgtgagccacggaagcggaggaagcggagagggcaggggaagtcttctaacatgcggggacgtggaggaaaatcccggccccatgatcaagatcgccaccaggaagtacctgggcaagcagaacgtgtacgacatcggcgtggagagggaccacaacttcgccctgaagaacggcttcatcgccagcaactgtagggagttgatttcagactatgcaccagattgttttgtgttaatagaaatgaatggcaagttagtcaaaactacgattgaagaactcattccactcaaatatacccgaaattaa

SEQ ID NO: 11 - DNA, 768 bp, synthetic construct
  misc_feature 1..768: note = pDeliver retargeting sequence (N-terminal BsrR - Npu DnaE-N -Furin-T2A - BXB1 attP)
  source 1..768: mol_type = other DNA
atgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctgagctacgagaccgagatcctgaccgtggagtacggcctgctgcccatcggcaagatcgtggagaagaggatcgagtgcaccgtgtacagcgtggacaacaacggcaacatctacacccagcccgtggcccagtggcacgacaggggcgagcaggaggtgttcgagtactgcctggaggacggcagcctgatcagggccaccaaggaccacaagttcatgaccgtggacggccagatgctgcccatcgacgagatcttcgagagggagctggacctgatgagggtggacaacctgcccaacggcatcaggaggaagaggagcgtgagccacggaagcggagagggcaggggaagtcttctaacatgcggggacgtggaggaaaatcccggccccgtcgtggtttgtctggtcaaccaccgcggtctcagtggtgtacggtacaaaccccgacggtaa

SEQ ID NO: 12 - DNA, 711 bp, synthetic construct
  misc_feature 1..711: note = mCherry coding sequence
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 13 - DNA, 1503 bp, Mycobacteriophage Bxb1
  source 1..1503: mol_type = genomic DNA
atgagggccctggtggtgatcaggctgagcagggtgaccgacgccaccaccagccccgagaggcagctggagagctgccagcagctgtgcgcccagaggggctgggacgtggtgggcgtggccgaggacctggacgtgagcggcgccgtggaccccttcgacaggaagaggaggcccaacctggccaggtggctggccttcgaggagcagcccttcgacgtgatcgtggcctacagggtggacaggctgaccaggagcatcaggcacctgcagcagctggtgcactgggccgaggaccacaagaagctggtggtgagcgccaccgaggcccacttcgacaccaccacccccttcgccgccgtggtgatcgccctgatgggcaccgtggcccagatggagctggaggccatcaaggagaggaacaggagcgccgcccacttcaacatcagggccggcaagtacaggggcagcctgcccccctggggctacctgcccaccagggtggacggcgagtggaggctggtgcccgaccccgtgcagagggagaggatcctggaggtgtaccacagggtggtggacaaccacgagcccctgcacctggtggcccacgacctgaacaggaggggcgtgctgagccccaaggactacttcgcccagctgcagggcagggagccccagggcagggagtggagcgccaccgccctgaagaggagcatgatcagcgaggccatgctgggctacgccaccctgaacggcaagaccgtgagggacgacgacggcgcccccctggtgagggccgagcccatcctgaccagggagcagctggaggccctgagggccgagctggtgaagaccagcagggccaagcccgccgtgagcacccccagcctgctgctgagggtgctgttctgcgccgtgtgcggcgagcccgcctacaagttcgccggcggcggcaggaagcaccccaggtacaggtgcaggagcatgggcttccccaagcactgcggcaacggcaccgtggccatggccgagtgggacgccttctgcgaggagcaggtgctggacctgctgggcgacgccgagaggctggagaaggtgtgggtggccggcagcgacagcgccgtggagctggccgaggtgaacgccgagctggtggacctgaccagcctgatcggcagccccgcctacagggccggcagcccccagagggaggccctggacgccaggatcgccgccctggccgccaggcaggaggagctggagggcctggaggccaggcccagcggctgggagtggagggagaccggccagaggttcggcgactggtggagggagcaggacaccgccgccaagaacacctggctgaggagcatgaacgtgaggctgaccttcgacgtgaggggcggcctgaccaggaccatcgacttcggcgacctgcaggagtacgagcagcacctgaggctgggcagcgtggtggagaggctgcacaccggcatgagctaa

SEQ ID NO: 14 - DNA, 5887 bp, synthetic construct
  misc_feature 1..5887: note = pDeliver mCherry construct
  source 1..5887: mol_type = other DNA
caactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagcatccgtttgcgtattgggcgctcttccgctgatctgcgcagcaccatgttctacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactgtcgggatcaccgaattcaccggtgccgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctgagctacgagaccgagatcctgaccgtggagtacggcctgctgcccatcggcaagatcgtggagaagaggatcgagtgcaccgtgtacagcgtggacaacaacggcaacatctacacccagcccgtggcccagtggcacgacaggggcgagcaggaggtgttcgagtactgcctggaggacggcagcctgatcagggccaccaaggaccacaagttcatgaccgtggacggccagatgctgcccatcgacgagatcttcgagagggagctggacctgatgagggtggacaacctgcccaacggcatcaggaggaagaggagcgtgagccacggaagcggagagggcaggggaagtcttctaacatgcggggacgtggaggaaaatcccggccccgtcgtggtttgtctggtcaaccaccgcggtctcagtggtgtacggtacaaaccccgacggtaaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgccgcggccgc

SEQ ID NO: 15 - DNA, 7105 bp, synthetic construct
  misc_feature 1..7105: note = pPlatform EGFP landing pad
  source 1..7105: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgccagatttgtgcatacacagtgactcatactttcaccaatactttgcattttggataaatactagacaactttagaagtgaattatttatgaggttgtcttaaaattaaaaattacaaagtaataaatcacattgtaatgtattttgtgtgatacccagaggtttaaggcaacctattactcttatgctcctgaagtccacaattcacagtcctgaactataatcttatctttgtgattgctgagcaaatttgcagtataatttcagtgcttttaaattttgtcctgcttactattttccttttttatttgggtttgatatgcgtgcacagaatggggcttctattaaaatattccatggcttacatttttaatattttgttctcttaatatgttcaaagctactcaacttttattcccgaaaaatgtttactttaattattctaatttcttacataaagcattgaggtgctaacaattatatactatgtacaagatggcagactaaatcatatcataccatcaagtagaaacctggagtttggtgaactttgagttgtttatatgtctctcctttattgtcttctcaaaacctgtgattctgaagtcaaagggacacagctgtcacatgaaaagtgatcacttatcacctgtatgcataaaacaccttaccaagcagctaagaggagtaactcctagccactttgagaaacgtttttgaataaacagagcaaggctcttccccattctcccagagatatagcataaaactgagcgcatttttataaaacaaaaaaggaggaatgtgtggtttgatggccagaccctgaatttgtgttcagcatctgcttttccatattatagatgggtaccagtgattctgagccatgtctatttctcctgacttttcctctgttttcccacgcttgctgatatttacagccgtggtcatcacaatcacctttgttcctttcttccttcctccaactctgcattaaattccaggaacttgctttctgtgaagtctaggggcgggactctggggttcgaaatgaccgaccaagcgacgcccaacctgccatcagtggtttgtctggtcaaccaccgcggtctcagtggtgtacggtacaaacccacaagtttgtacaaaaaagcaggctgccacctgataatagctgataatagtcggccggcttgtcgacgacggcggtctccgtcgtcaggatcatccgggcggcatcaggaggaagaggagcgtgagccacggaagcggaggaagcggagagggcaggggaagtcttctaacatgcggggacgtggaggaaaatcccggccccatgatcaagatcgccaccaggaagtacctgggcaagcagaacgtgtacgacatcggcgtggagagggaccacaacttcgccctgaagaacggcttcatcgccagcaactgtagggagttgatttcagactatgcaccagattgttttgtgttaatagaaatgaatggcaagttagtcaaaactacgattgaagaactcattccactcaaatatacccgaaattaacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtggccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 16 - DNA, 1179 bp, Homo sapiens
  source 1..1179: mol_type = genomic DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 17 - DNA, 500 bp, Mus musculus
  source 1..500: mol_type = genomic DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 18 - DNA, 508 bp, Human betaherpesvirus 5 (CMV)
  source 1..508: mol_type = genomic DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 19 - skipped

SEQ ID NO: 20 - DNA, 19 bp, synthetic construct
  misc_feature 1..19: note = 2 x 3 stop codon
  source 1..19: mol_type = other DNA
tgataatagctgataatag

SEQ ID NO: 21 - protein, 361 aa, synthetic construct
  REGION 1..361: note = split BsrR including inteins (protein sequence from retargeted nucleic acid)
MKTFNISQQDLELVEVATEKITMLYEDNKHHVGAAIRTKTGEIISAVHIEAYIGRVTVCAEAIAIGSAVSNGQKDFDTIVAVRHPYSDEVDRSIRVVSPCGMCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGIRRKRSVSHGSGEGRGSLLTCGDVEENPGPVVVCLVNHRGLRRQDHPGGIRRKRSVSHGSGGSGEGRGSLLTCGDVEENPGPMIKIATRKYLGKQNVYDIGVERDHNFALKNGFIASNCRELISDYAPDCFVLIEMNGKLVKTTIEELIPLKYTRN

SEQ ID NO: 22 - protein, 16 aa, synthetic construct
  REGION 1..16: note = Furin-T2A linker protein sequence (pPlatform)
GIRRKRSVSHGSGGSG

SEQ ID NO: 23 - protein, 13 aa, synthetic construct
  REGION 1..13: note = Furin-T2A linker protein sequence (pDeliver)
GIRRKRSVSHGSG

SEQ ID NO: 24 - DNA, 344 bp, synthetic construct
  source 1..344: mol_type = other DNA
  misc_feature 1..344: note = SV40 promoter
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 25 - DNA, 774 bp, synthetic construct
  source 1..774: mol_type = other DNA
  misc_feature 1..774: note = FOLR1A coding sequence
atggcccagaggatgaccacccagctgctgctgctgctggtgtgggtggccgtggtgggcgaggcccagaccaggatcgcctgggccaggaccgagctgctgaacgtgtgcatgaacgccaagcaccacaaggagaagcccggccccgaggacaagctgcacgagcagtgcaggccctggaggaagaacgcctgctgcagcaccaacaccagccaggaggcccacaaggacgtgagctacctgtacaggttcaactggaaccactgcggcgagatggcccccgcctgcaagaggcacttcatccaggacacctgcctgtacgagtgcagccccaacctgggcccctggatccagcaggtggaccagagctggaggaaggagagggtgctgaacgtgcccctgtgcaaggaggactgcgagcagtggtgggaggactgcaggaccagctacacctgcaagagcaactggcacaagggctggaactggaccagcggcttcaacaagtgcgccgtgggcgccgcctgccagcccttccacttctacttccccacccccaccgtgctgtgcaacgagatctggacccacagctacaaggtgagcaactacagcaggggcagcggcaggtgcatccagatgtggttcgaccccgcccagggcaaccccaacgaggaggtggccaggttctacgccgccgccatgagcggcgccggcccctgggccgcctggcccttcctgctgagcctggccctgatgctgctgtggctgctgagctga

SEQ ID NO: 26 - DNA, 999 bp, synthetic construct
  source 1..999: mol_type = other DNA
  misc_feature 1..999: note = CD19 coding sequence
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagatga

SEQ ID NO: 27 - DNA, 5079 bp, synthetic construct
  source 1..5079: mol_type = other DNA
  misc_feature 1..5079: note = pDeliver FOLR1A construct
caactttgtatagaaaagttgggcctaactggccggtaccgagtttctagacggagtactgtcctccgagcggagtactgtcctccgactcgagcggagtactgtcctccgagcggagtactgtcctccgagcggagtactgtcctccgagcggagtactgtcctccgagcggagtactgtcctccgagcggagtactgtcctccgaggaattccggagtactgtcctccgaagacgctagcggggggctataaaagggggtgggggcgttcgtcctcactctagatctgcgatctaagtaagcttggcaatccggtactgttggtaaacaagtttgtacaaaaaagcaggctgccaccatggcccagaggatgaccacccagctgctgctgctgctggtgtgggtggccgtggtgggcgaggcccagaccaggatcgcctgggccaggaccgagctgctgaacgtgtgcatgaacgccaagcaccacaaggagaagcccggccccgaggacaagctgcacgagcagtgcaggccctggaggaagaacgcctgctgcagcaccaacaccagccaggaggcccacaaggacgtgagctacctgtacaggttcaactggaaccactgcggcgagatggcccccgcctgcaagaggcacttcatccaggacacctgcctgtacgagtgcagccccaacctgggcccctggatccagcaggtggaccagagctggaggaaggagagggtgctgaacgtgcccctgtgcaaggaggactgcgagcagtggtgggaggactgcaggaccagctacacctgcaagagcaactggcacaagggctggaactggaccagcggcttcaacaagtgcgccgtgggcgccgcctgccagcccttccacttctacttccccacccccaccgtgctgtgcaacgagatctggacccacagctacaaggtgagcaactacagcaggggcagcggcaggtgcatccagatgtggttcgaccccgcccagggcaaccccaacgaggaggtggccaggttctacgccgccgccatgagcggcgccggcccctgggccgcctggcccttcctgctgagcctggccctgatgctgctgtggctgctgagctgaggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagcatccgtttgcgtattgggcgctcttccgctgatctgcgcagcaccatgttctacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactgtcgggatcaccgaattcaccggtgccgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctgagctacgagaccgagatcctgaccgtggagtacggcctgctgcccatcggcaagatcgtggagaagaggatcgagtgcaccgtgtacagcgtggacaacaacggcaacatctacacccagcccgtggcccagtggcacgacaggggcgagcaggaggtgttcgagtactgcctggaggacggcagcctgatcagggccaccaaggaccacaagttcatgaccgtggacggccagatgctgcccatcgacgagatcttcgagagggagctggacctgatgagggtggacaacctgcccaacggcatcaggaggaagaggagcgtgagccacggaagcggagagggcaggggaagtcttctaacatgcggggacgtggaggaaaatcccggccccgtcgtggtttgtctggtcaaccaccgcggtctcagtggtgtacggtacaaaccccgacggtaaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgccgcggccgc

SEQ ID NO: 28 - DNA, 5304 bp, synthetic construct
  source 1..5304: mol_type = other DNA
  misc_feature 1..5304: note = pDeliver CD19 construct
caactttgtatagaaaagttgggcctaactggccggtaccgagtttctagacggagtactgtcctccgagcggagtactgtcctccgactcgagcggagtactgtcctccgagcggagtactgtcctccgagcggagtactgtcctccgagcggagtactgtcctccgagcggagtactgtcctccgagcggagtactgtcctccgaggaattccggagtactgtcctccgaagacgctagcggggggctataaaagggggtgggggcgttcgtcctcactctagatctgcgatctaagtaagcttggcaatccggtactgttggtaaacaagtttgtacaaaaaagcaggctgccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagatgaggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagcatccgtttgcgtattgggcgctcttccgctgatctgcgcagcaccatgttctacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactgtcgggatcaccgaattcaccggtgccgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctgagctacgagaccgagatcctgaccgtggagtacggcctgctgcccatcggcaagatcgtggagaagaggatcgagtgcaccgtgtacagcgtggacaacaacggcaacatctacacccagcccgtggcccagtggcacgacaggggcgagcaggaggtgttcgagtactgcctggaggacggcagcctgatcagggccaccaaggaccacaagttcatgaccgtggacggccagatgctgcccatcgacgagatcttcgagagggagctggacctgatgagggtggacaacctgcccaacggcatcaggaggaagaggagcgtgagccacggaagcggagagggcaggggaagtcttctaacatgcggggacgtggaggaaaatcccggccccgtcgtggtttgtctggtcaaccaccgcggtctcagtggtgtacggtacaaaccccgacggtaaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgccgcggccgc

(19) United States
(12) Patent Application Publication
(10) Pub. No.: US 2026/0092272 A1
(43) Pub. Date: Apr. 2, 2026

(54) Integration and Selection System

(71) Applicant: Antibody Analytics Limited, Motherwell (GB)

(72) Inventors: Timothy London, Motherwell (GB); Hannah Findlay, Motherwell (GB); Amy Graham, Motherwell (GB); Kieran Izzett, Motherwell (GB); Agapitos Patakas, Motherwell (GB)

(73) Assignee: Antibody Analytics Limited, Motherwell (GB)

(21) Appl. No.: 19/113,639

(22) PCT Filed: Sep. 20, 2023
(86) PCT No.: PCT/GB2023/052443

§ 371 Date: Mar. 20, 2025

(30) Foreign Application Priority Data
Sep. 21, 2022 (EP) 22386064.4

(51) Int. Cl. C12N 15/10; C40B 30/06; C40B 50/06

(52) U.S. Cl. CPC C12N 15/1082; C40B 30/06; C40B 50/06

(57) ABSTRACT

The present invention relates to a system comprising a landing pad, a delivery nucleic acid and an integrating enzyme allowing selection of a specific gene of interest. The invention further relates to cells comprising the system, a method for providing a gene of interest and selecting a cell with a gene of interest, a landing pad nucleic acid, a delivery nucleic acid and a kit for providing and selecting a cell with a gene of interest.

### FIELD OF THE INVENTION

The present invention relates to a system comprising a landing pad, a delivery nucleic acid and an integrating enzyme allowing selection of a specific gene of interest. The invention further relates to cells comprising the system, a method for providing a gene of interest and selecting a cell with a gene of interest, a landing pad nucleic acid, a delivery nucleic acid and a kit for providing and selecting a cell with a gene of interest.

### INTRODUCTION

Many of the current and emerging therapeutics are therapeutic proteins such as monoclonal antibodies, peptides and recombinant proteins. Production of such therapeutic proteins is particularly challenging as therapeutic proteins require complex post-translational modifications so they can function. In order to ensure the proper post-translational modifications are created during production, often mammalian cells are used for production of such proteins.

To facilitate continuous production of therapeutic proteins, stable producer cell lines are commonly generated. However, the process of generating a stable producer cell line is time consuming and expensive since a large number of clones need to be screened. Therefore, there is a need for a system for generating a stable producer cell line in a quick and cost-efficient manner for example by reducing the number of clones which need to be screened.

### SUMMARY OF THE INVENTION

In a first aspect of the invention, there is provided a system comprising:
a landing pad nucleic acid comprising in the 5′ to 3′ direction: an expression stop signal, a first site-specific recombination site, a first transcriptional or translational split mechanism, a first split intein and split selectable marker combination;
a delivery nucleic acid comprising in the 5′ to 3′ direction: a second split intein and split selectable marker combination, a second transcriptional or translational split mechanism and a second site-specific recombination site, wherein the delivery nucleic acid further comprises a first promoter operably linked to a gene of interest; and
an integrating enzyme, or a nucleic acid encoding an integrating enzyme, wherein the integrating enzyme is configured to catalyse site specific recombination between the first site-specific recombination site and the second site-specific recombination site.

In some embodiments, the first split intein and split selectable marker combination comprises in the 5′ to 3′ direction a N-terminal part of the split selectable marker and a N-terminal part of the split intein. In some embodiments, the second split intein and split selectable marker combination comprises in the 5′ to 3′ direction a C-terminal part of a split intein, and a C-terminal part of a split selectable marker. In some embodiments, when the first split intein and split selectable marker combination comprises in the 5′ to 3′ direction N-terminal part of the split selectable marker and a N-terminal part of the split intein, the second split intein and split selectable marker combination comprises in the 5′ to 3′ direction C-terminal part of a split intein, and a C-terminal part of a split selectable marker. In some embodiments, the system comprises:
a landing pad nucleic acid comprising in the 5′ to 3′ direction: an expression stop signal, a first site-specific recombination site, a first transcriptional or translational split mechanism, N-terminal part of the split selectable marker, and a N-terminal part of the split intein;
a delivery nucleic acid comprising in the 5′ to 3′ direction: a C-terminal part of a split intein, and a C-terminal part of a split selectable marker, a second transcriptional or translational split mechanism and a second site-specific recombination site, wherein the delivery nucleic acid further comprises a first promoter operably linked to a gene of interest; and
an integrating enzyme, or a nucleic acid encoding an integrating enzyme, wherein the integrating enzyme is configured to catalyse site specific recombination between the first site-specific recombination site and the second site-specific recombination site.

In some embodiments, the first split intein and split selectable marker combination comprises in the 5′ to 3′ direction a C-terminal part of a split intein, and a C-terminal part of a split selectable marker. In some embodiments, the second split intein and split selectable marker combination comprises in the 5′ to 3′ direction an N-terminal part of the split selectable marker, an N-terminal part of the split intein. In some preferred embodiments, when the first split intein and split selectable marker combination comprises in the 5′ to 3′ direction C-terminal part of a split intein and a C-terminal part of a split selectable marker, the second split intein and split selectable marker combination comprises in the 5′ to 3′ direction N-terminal part of the split selectable marker and a N-terminal part of the split intein. In some preferred embodiments, the system comprises:
a landing pad nucleic acid comprising in the 5′ to 3′ direction: an expression stop signal, a first site-specific recombination site, a first transcriptional or translational split mechanism, a C-terminal part of a split intein, and a C-terminal part of a split selectable marker;
a delivery nucleic acid comprising in the 5′ to 3′ direction: an N-terminal part of the split selectable marker, an N-terminal part of the split intein, a second transcriptional or translational split mechanism and a second site-specific recombination site, wherein the delivery nucleic acid further comprises a first promoter operably linked to a gene of interest; and
an integrating enzyme, or a nucleic acid encoding an integrating enzyme, wherein the integrating enzyme is configured to catalyse site specific recombination between the first site-specific recombination site and the second site-specific recombination site.

In some embodiments, the C-terminal part of the split intein and the N-terminal part of the split intein are configured to recombine the C-terminal part of the split selectable marker and the N-terminal part of the split selectable marker into a functional selectable marker.

The landing pad nucleic acid may be for integration in a genome of a cell. The landing pad nucleic acid may be configured to integrate in a genome of a cell. Once the landing pad nucleic acid has been integrated into the genome of the cell, a landing pad cell line is generated. The landing pad cell line can be used as a platform for subsequent integration of a delivery nucleic acid. For example, one landing pad cell line may be used for subsequent integration of different delivery nucleic acids with different genes of interest (e.g. a population of cells from the landing pad cell line may be used for integration of a delivery nucleic acid with a first gene of interest and another population of cells from the landing pad cell line may be used for integration of a different delivery nucleic acid with a second gene of interest). The system according to the present invention may be used to generate a landing pad cell line.

The system according to the present invention may be used to generate a cell line which expresses the gene of interest. The delivery nucleic acid may be for integration of a gene of interest into the landing pad nucleic acid. The delivery nucleic acid may be configured to integrate a gene of interest into the landing pad nucleic acid. In some embodiments, integration of the landing pad nucleic acid into the genome of interest and subsequent integration of the delivery nucleic acid into the landing pad nucleic acid results in reconstruction of the selectable marker following translation of the parts of the split selectable marker. One of the benefits of the present system is that in the absence of subsequent integration of the delivery nucleic acid into the landing pad nucleic acid, the selectable marker cannot be reconstructed as only the C-terminal part of a split selectable marker (as shown in FIG. 1A) or only the N-terminal part of a split selectable marker (as shown in FIG. 4A) would be present in the cell. In some embodiments, the expression stop signal within the landing pad nucleic acid ensure that the first split intein and split selectable marker combination (for example, C-terminal part of a split intein and the C-terminal part of a split selectable marker or N-terminal part of the split selectable marker, and a N-terminal part of the split intein) cannot be expressed from the landing pad nucleic acid alone (i.e., without integration of the delivery nucleic acid within the landing pad nucleic acid).

One of the benefits of the present system is that only integration of the delivery nucleic acid in the correct position and orientation within the landing pad nucleic acid will result in reconstruction of the selectable marker following translation of the parts of the split selectable marker. This in turn will reduce the number of clones which need to be screened as part of generating a stable cell line and will reduce the time and cost involved in generating a stable cell line.

In some embodiments, the second split intein and split selectable marker combination is operably linked to a second promoter. In some embodiments, the second split intein and split selectable marker combination is operably linked to the first promoter which controls expression of the gene of interest. In such embodiments, the second split intein and split selectable marker combination may be split from the gene of interest via a third transcriptional or translational split mechanism. The third transcriptional or translational split mechanism may be an internal ribosome entry site (IRES), furin cleavage site or a 2A peptide. In some preferred embodiments, the third transcriptional or translational split mechanism is an internal ribosome entry site (IRES).

In some embodiments, the C-terminal part of the split intein and the C-terminal part of the split selectable marker is operably linked to a second promoter. In some embodiments, the C-terminal part of the split intein and the C-terminal part of the split selectable marker is operably linked to the first promoter which controls expression of the gene of interest. In such embodiments, the C-terminal part of the split intein and the C-terminal part of the split selectable marker may be split from the gene of interest via a third transcriptional or translational split mechanism. The third transcriptional or translational split mechanism may be an internal ribosome entry site (IRES), furin cleavage site or a 2A peptide. In some preferred embodiments, the third transcriptional or translational split mechanism is an internal ribosome entry site (IRES).

In some embodiments, the N-terminal part of the split selectable marker and the N-terminal part of the split intein is operably linked to a second promoter. In some embodiments, the N-terminal part of the split selectable marker and the N-terminal part of the split intein is operably linked to the first promoter which controls expression of the gene of interest. In such embodiments, the N-terminal part of the split selectable marker and the N-terminal part of the split intein may be split from the gene of interest via a third transcriptional or translational split mechanism. The third transcriptional or translational split mechanism may be an internal ribosome entry site (IRES), furin cleavage site or a 2A peptide. In some preferred embodiments, the third transcriptional or translational split mechanism is an internal ribosome entry site (IRES).

In some embodiments, following the catalysation of site specific recombination between the first site-specific recombination site and the second site-specific recombination site by the integrating enzyme, the resultant nucleic acid (herein called retargeted nucleic acid) comprises in the 5′ to 3′ direction: an expression stop signal, a first promoter operably linked to a gene of interest, a second promoter operably linked to a N-terminal part of the split 20 selectable marker, a N-terminal part of the split intein, a second transcriptional or translational split mechanism, a first transcriptional or translational split mechanism, a C-terminal part of a split intein, and a C-terminal part of a split selectable marker. The retargeted nucleic acid may further comprise a first site-specific recombination site and/or a second site-specific recombination site or one or more nucleic acid sequences resulting from recombination of the first site-specific recombination site and the second site-specific recombination site. The retargeted nucleic acid may further comprise attL and attR (see below). In some embodiments, the retargeted nucleic acid comprises in the 5′ to 3′ direction: an expression stop signal, attL, a first promoter operably linked to a gene of interest, a second promoter operably linked to a N-terminal part of the split selectable marker, a N-terminal part of the split intein, a second transcriptional or translational split mechanism, attR, a first transcriptional or translational split mechanism, a C-terminal part of a split intein, and a C-terminal part of a split selectable marker. In some embodiments, the retargeted nucleic acid comprises or consists of SEQ ID NO: 11 or SEQ ID NO: 21 or a functional variant thereof.

In some embodiments, following the catalysation of site specific recombination between the first site-specific recombination site and the second site-specific recombination site by the integrating enzyme, the resultant nucleic acid (herein called retargeted nucleic acid) comprises in the 5′ to 3′ direction: an expression stop signal, a first promoter operably linked to a gene of interest, a second promoter operably linked to a C-terminal part of a split intein, and a C-terminal part of a split selectable marker, a first transcriptional or translational split mechanism, a second transcriptional or translational split mechanism, a N-terminal part of the split selectable marker, a N-terminal part of the split intein. The retargeted nucleic acid may further comprise a first site-specific recombination site and/or a second site-specific recombination site or one or more nucleic acid sequences resulting from recombination of the first site-specific recombination site and the second site-specific recombination site. The retargeted nucleic acid may further comprise attL and attR (see below). In some embodiments, the retargeted nucleic acid comprises in the 5′ to 3′ direction: an expression stop signal, attL, a first promoter operably linked to a gene of interest, a second promoter operably linked to a C-terminal part of a split intein, and a C-terminal part of a split selectable marker, a first transcriptional or translational split mechanism, attR, a second transcriptional or translational split mechanism, a N-terminal part of the split selectable marker, a N-terminal part of the split intein. In some embodiments, following the catalysation of site specific recombination between the first site-specific recombination site and the second site-specific recombination site by the integrating enzyme, the resultant nucleic acid (herein called retargeted nucleic acid) comprises in the 5′ to 3′ direction: an expression stop signal, a first promoter operably linked to a gene of interest, a third transcriptional or translational split mechanism, a N-terminal part of the split selectable marker, a N-terminal part of the split intein, a second transcriptional or translational split mechanism, a first transcriptional or translational split mechanism, a C-terminal part of a split intein, and a C-terminal part of a split selectable marker. The retargeted nucleic acid may further comprise a first site-specific recombination site and/or a second site-specific recombination site or one or more nucleic acid sequences resulting from recombination of the first site-specific recombination site and the second site-specific recombination site. The retargeted nucleic acid may further comprise attL and attR (see below). In some embodiments, the retargeted nucleic acid comprises in the 5′ to 3′ direction: an expression stop signal, attL, a first promoter operably linked to a gene of interest, a third transcriptional or translational split mechanism, N-terminal part of the split selectable marker, a N-terminal part of the split intein, a second transcriptional or translational split mechanism, attR, a first transcriptional or translational split mechanism, a C-terminal part of a split intein, and a C-terminal part of a split selectable marker.

In some embodiments, the integrating enzyme is configured to catalyse site specific recombination between the first site-specific recombination site and the second site-specific recombination site. In some embodiments, the integrating enzyme is configured to catalyse site specific recombination between the first site-specific recombination site and the second site-specific recombination site resulting in integration of the delivery nucleic acid in the landing pad nucleic acid. In some embodiments, the integrating enzyme is a unidirectional integrase. In some embodiments, the integrating enzyme is a serine integrase. In some embodiments, the integrase is a unidirectional serine integrase. Unidirectional serine integrases are phage-encoded recombinases which promote conservative recombination between two short DNA fragments-phage attachment site, attP and bacterial attachment site, attB. The product of the recombination between the attP and attB is integration of a nucleic acid of interest flanked by two new recombination sites-attL and attR. Each of attL and attR contain half sites derived from attP and attB. Unidirectional serine integrases result in unidirectional integration, that is to say irreversible integration. Unidirectional integration may be particularly preferred in generating a stable producer cell line expressing a gene of interest as it cannot be reversed by subsequent integration events.

The integrating enzyme may be selected from the group of unidirectional serine integrases consisting of: Bxb1, Wβ, BL3, R4, A118, TG1, MR11, φ370, SPBc, TP901-1, φRV, FC1, K38, φBT1 and φC31. The integrating enzyme may be selected from the group of unidirectional serine integrases consisting of: Bxb1, φC31, R4 and φBT1. In some preferred embodiments, the integrating enzyme is BxB1. BXB1 has been found to be functional in a broad range of mammalian cells and is typically considered to be the most efficient and most accurate out of the currently known unidirectional serine integrases. In some embodiments, the nucleic acid encoding the integrating enzyme comprises or consists of SEQ ID NO: 13 or a functional variant thereof.

In some embodiments, the first site-specific recombination site is the attB of the respective unidirectional serine integrase. For example, when the integrating enzyme is BXB1, the first site-specific recombination site is BXB1 attB. Suitably, the first site-specific recombination site may be selected from the group consisting of: Bxb1 attB, Wβ attB, BL3 attB, R4 attB, A118 attB, TG1 attB, MR11 attB, C370 attB, SPBc attB, TP901 attB, RV attB, FC1 attB, φK38 attB, φBT1 attB and C31 attB. The sequences of these recombination sites can be found in table 2 of Xu, Z., Thomas, L., Davies, B. et al. Accuracy and efficiency define Bxb1 integrase as the best of fifteen candidate serine recombinases for the integration of DNA into the human genome. BMC Biotechnol 13, 87 (2013). https://doi.org/10.1186/1472-6750-13-87 which is incorporated herein by reference. In some embodiments, the first site-specific recombination site is BXB1 attB. In some embodiments, the first site-specific recombination site comprises or consists of SEQ ID NO: 1 or a functional variant thereof.

In some embodiments, the second site-specific recombination site is the attP of the respective unidirectional serine integrase. For example, when the integrating enzyme is BXB1, the second site-specific recombination site is BXB1 attP. Suitably, the second site-specific recombination site is selected from the group consisting of: Bxb attP, Wβ attP, BL3 attP, R4 attP, A118 attP, TG1 attP, MR11 attP, φC370 attP, SPBc attP, TP901 attP, RV attP, FC1 attP, φK38 attP, φBT1 attP and C31 attP. In some embodiments, the second site-specific recombination site is Bxb attP. In some embodiments, the Bxb1 attP further comprises an in frame stop codon. The stop codon within the BxB1 attP may be added in order to terminate the translation from the N-terminal part or the C-terminal part of the split selectable marker in the delivery nucleic acid. In the retargeted construct, this stop codon moves outside of the coding sequences. In some embodiments, the second site-specific recombination site comprises or consists of SEQ ID NO: 2 or a functional variant thereof.

In some embodiments, the first and second site-specific recombination sites may be reversed, suitably such that the first site-specific recombination site may be the attB of the respective unidirectional serine integrase, and the second site-specific recombination site may be the attP of the respective unidirectional serine integrase. Suitably the attB and attP sites are as defined above.

The integrating enzyme may be a tyrosine recombinase. Suitably, the tyrosine recombinase may be Cre recombinase. Suitably, when the integrating enzyme is Cre recombinase, the first site-specific recombination site is LoxP. Suitably, when the integrating enzyme is Cre recombinase, the second site-specific recombination site is LoxP. Suitably, the tyrosine recombinase may be Flp. Suitably, when the integrating enzyme is Flp recombinase, the first site-specific recombination site is FRT. Suitably, when the integrating enzyme is Flp recombinase, the second site-specific recombination site is FRT.

The C-terminal part of the split intein may be the C-terminal part of NpuDnaE, SspDnaB or SspDnaE intein. In some embodiments, the C-terminal part of the split intein is the C-terminal part of the NpuDnaE intein. In some embodiments, the C-terminal part of the split intein comprises or consists of SEQ ID NO: 4 or a functional variant thereof.

The N-terminal part of a split intein may be the N-terminal part of NpuDnaE, SspDnaB or SspDnaE intein. In some embodiments, the N-terminal part of the split intein is the N-terminal part of the NpuDnaE intein. In some embodiments, the N-terminal part of the split intein comprises or consists of SEQ ID NO: 5 or a functional variant thereof.

The C-terminal part of the split intein and the N-terminal part of the split intein may be any pair of split inteins which are capable of enabling the ligation of flanking exteins into a new protein. The C-terminal part of the split intein and the N-terminal part of the split intein may be any pair of split inteins which are configured for protein splicing. The C-terminal part of the split intein and the N-terminal part of the split intein may be any pair of split inteins which are configured to recombine the flanking exteins into a new protein. The C-terminal part of the split intein and the N-terminal part of the split intein may be any pair of split inteins which are configured to recombine the C-terminal part of a split selectable marker and the N-terminal part of a split selectable marker into a functional selectable marker. The C-terminal part of the split intein may be the C-terminal part of SspDnaB intein and the N-terminal part of a split intein may be the N-terminal part of SspDnaB intein. The C-terminal part of the split intein may be the C-terminal part of SspDnaE intein and the N-terminal part of a split intein may be the N-terminal part of SspDnaE intein. In some embodiments, the C-terminal part of the split intein is the C-terminal part of NpuDnaE intein and the N-terminal part of a split intein is the N-terminal part of NpuDnaE intein. In some embodiments, the C-terminal part of the split intein comprises or consists of SEQ ID NO: 4 or a functional variant thereof and the N-terminal part of the split intein comprises or consists of SEQ ID NO: 5 or a functional variant thereof.

The first transcriptional or translational split mechanism may allow two separate proteins to be created from a single nucleic acid. The first transcriptional or translational split mechanism may allow two separate proteins to be created from a single nucleic acid via a transcriptional mechanism. For example, the nucleic acids encoding each of the proteins may be operably linked to separate promoters. The first transcriptional or translational split mechanism may allow two separate proteins to be created from a single nucleic acid via a translational mechanism. The translational mechanism may be during translation or post-translational. The first transcriptional or translational split mechanism may allow two separate proteins to be created from a single nucleic acid by allowing translation initiation in a cap-independent manner. The first transcriptional or translational split mechanism may allow two separate proteins to be created from a single nucleic acid by inducing ribosomal skipping during translation. The first transcriptional or translational split mechanism may be a DNA sequence, an RNA sequence or a protein sequence.

The first transcriptional or translational split mechanism may be an internal ribosome entry site (IRES), furin cleavage site or a 2A peptide. In some embodiments, the first transcriptional or translational split mechanism is an IRES. In some embodiments, the first transcriptional or translational split mechanism comprises or consists of SEQ ID NO: 8 or a functional variant thereof. In some embodiments, the first transcriptional or translational split mechanism is a cleavable peptide such as a 2A peptide. The first transcriptional or translational split mechanism may be a 2A peptide selected from the group consisting of: F2A, P2A, E2A, T2A, GF2A, GP2A, GE2A and GT2A. In some embodiments, the first transcriptional or translational split mechanism is a T2A peptide.

In some embodiments, the first transcriptional or translational split mechanism is a 2A peptide with a furin recognition site. A furin recognition site, also known as furin cleavage site, is a protein sequence which is predicted to be recognised and cleaved by the protease enzyme furin. A furin recognition site may be used to remove any remaining appended 2A sequence. A furin recognition site may also be used to supplement the efficiency of the 2A cleavage. In some preferred embodiments, the first transcriptional or translational split mechanism is a T2A peptide with a furin recognition site. In some embodiments, the first transcriptional or translational split mechanism comprises or consist of SEQ ID NO: 3, SEQ ID NO: 22 or a functional variant thereof.

The second transcriptional or translational split mechanism may allow two separate proteins to be created from a single nucleic acid. The second transcriptional or translational split mechanism may allow two separate proteins to be created from a single nucleic acid via a transcriptional mechanism. For example, the nucleic acids encoding each of the proteins may be operably linked to separate promoters. The second transcriptional or translational split mechanism may allow two separate proteins to be created from a single nucleic acid via translational mechanism. The second transcriptional or translational split mechanism may allow two separate proteins to be created from a single nucleic acid via a translational mechanism. The translational mechanism may be during translation or post-translational.

The second transcriptional or translational split mechanism may allow two separate proteins to be created from a single nucleic acid by allowing translation initiation in a cap-independent manner. The second transcriptional or translational split mechanism may allow two separate proteins to be created from a single nucleic acid by inducing ribosomal skipping during translation. The second transcriptional or translational split mechanism may be a DNA sequence, an RNA sequence or a protein sequence.

The second transcriptional or translational split mechanism may be an internal ribosome entry site (IRES), furin cleavage site or a 2A peptide. In some embodiments, the second transcriptional or translational split mechanism is an IRES. In some embodiments, the second transcriptional or translational split mechanism comprises or consists of SEQ ID NO: 8 or a functional variant thereof. In some embodiments, the second transcriptional or translational split mechanism is a cleavable peptide, such as a 2A peptide. The second transcriptional or translational split mechanism may be a 2A peptide selected from the group consisting of: F2A, P2A, E2A, T2A, GF2A, GP2A, GE2A and GT2A. In some embodiments, the first transcriptional or translational split mechanism is a T2A peptide.

In some embodiments, the second transcriptional or translational split mechanism is a 2A peptide with a furin recognition site. A furin recognition site may be used to remove any remaining appended 2A sequence. A furin recognition site may also be used to supplement the efficiency of the 2A cleavage. In some preferred embodiments, the second transcriptional or translational split mechanism is a T2A peptide with a furin recognition site. In some embodiments, the second transcriptional or translational split mechanism comprises or consist of SEQ ID NO: 3, SEQ ID NO: 23 or a functional variant thereof.

In some embodiments, the first transcriptional or translational split mechanism is a 2A peptide with a furin recognition site and the second transcriptional or translational split mechanism is a 2A peptide with a furin recognition site. In some embodiments, the first transcriptional or translational split mechanism is a T2A peptide with a furin recognition site and the second transcriptional or translational split mechanism is a T2A peptide with a furin recognition site. In some embodiments, the first transcriptional or translational split mechanism comprises or consist of SEQ ID NO: 3 or SEQ ID NO: 22 or a functional variant thereof and the second transcriptional or translational split mechanism comprises or consist of SEQ ID NO: 3 or SEQ ID NO: 23 or a functional variant thereof.

The expression stop signal may be any sequence which prevents expression of the first site-specific recombination site, the first transcriptional or translational split mechanism, the C-terminal part of a split intein, and the C-terminal part of a split selectable marker from the landing pad nucleic acid or which prevents expression of the first site-specific recombination site, the first transcriptional or translational split mechanism, N-terminal part of the split selectable marker, and a N-terminal part of the split intein from the landing pad nucleic acid.

The expression stop signal may be an insulator sequence. The expression stop signal may be a polyA sequence. In some preferred embodiments, the expression stop signal is one or more stop codons. The one or more stop codons may be, for example, 2, 3, 4, 5, 6, 7, 8, 9, or 10 stop codons. A stop codon is a sequence of three nucleotides in DNA or messenger RNA which signal an end to protein synthesis. UAA, UAG and UGA are stop codons in RNA. TAA, TAG and TGA are stop codons in DNA. In some embodiments, the one or more stop codons are two sets of three stop codons. In some embodiments, the one or more stop codons comprise or consist of SEQ ID NO: 19 or a functional variant thereof. In some embodiments, the one or more stop codons comprise or consist of SEQ ID NO: 20 or a functional variant thereof.

The split selectable marker may be any suitable marker gene which expresses a marker that is readily detectable, many such marker genes are well-known in the art. For example, the selectable marker gene may be an antibiotic resistance gene, a gene encoding a fluorescent protein, a gene encoding glutamine synthetase or a gene encoding a luminescent protein. The split selectable marker may be a gene encoding a fluorescent protein. A fluorescent protein allows selection based on the presence or absence of a fluorescent protein (e.g. by FACS). The split selectable marker may be a gene encoding glutamine synthetase. Glutamine synthetase allows selection based on the ability of a cell to produce glutamine which is an essential amino acid required for cell survival. Some mammalian cells are not able to produce glutamine. For other cells an inhibitor of glutamine synthase (MSX) may be used. The split selectable marker may be a gene encoding a luminescent protein. A luminescent protein allows selection based on the presence or absence of a luminescent protein.

In some embodiments, the split selectable marker is an antibiotic resistance gene. An antibiotic resistance gene allows selection based on the ability of a cell to defeat an antibiotic. Antibiotic resistance gene is a particularly preferred split selectable marker as it allows for an efficient selection process as cells are simply grown in a substrate which includes the antibiotic for the respective antibiotic resistance gene. Suitably the split selectable marker may be split at any position in the amino acid sequence thereof, suitably to create a C-terminal part and an N-terminal part.

The C-terminal part of the split selectable marker and the N-terminal part of the split selectable marker may be the C-terminal part and the N-terminal part of a hygromycin resistance gene (HygroR) split at amino acid position 52. The C-terminal part of the split selectable marker and the N-terminal part of the split selectable marker may be the C-terminal part and the N-terminal part of a hygromycin resistance gene (HygroR) split at amino acid position 69. The C-terminal part of the split selectable marker and the N-terminal part of the split selectable marker may be the C-terminal part and the N-terminal part of a hygromycin resistance gene (HygroR) split at amino acid position 89.

The C-terminal part of the split selectable marker and the N-terminal part of the split selectable marker may be the C-terminal part and the N-terminal part of a hygromycin resistance gene (HygroR) split at amino acid position 131. The C-terminal part of the split selectable marker and the N-terminal part of the split selectable marker may be the C-terminal part and the N-terminal part of a hygromycin resistance gene (HygroR) split at amino acid position 171. The C-terminal part of the split selectable marker and the N-terminal part of the split selectable marker may be the C-terminal part and the N-terminal part of a hygromycin resistance gene (HygroR) split at amino acid position 200.

The C-terminal part of the split selectable marker and the N-terminal part of the split selectable marker may be the C-terminal part and the N-terminal part of a hygromycin resistance gene (HygroR) split at amino acid position 240. The C-terminal part of the split selectable marker and the N-terminal part of the split selectable marker may be the C-terminal part and the N-terminal part of a hygromycin resistance gene (HygroR) split at amino acid position 292.

The C-terminal part of the split selectable marker and the N-terminal part of the split selectable marker may be the C-terminal part and the N-terminal part of a puromycin resistance gene (PuroR) split at amino acid position 32. The C-terminal part of the split selectable marker and the N-terminal part of the split selectable marker may be the C-terminal part and the N-terminal part of a puromycin resistance gene (PuroR) split at amino acid position 84. The C-terminal part of the split selectable marker and the N-terminal part of the split selectable marker may be the C-terminal part and the N-terminal part of a puromycin resistance gene (PuroR) split at amino acid position 100. The C-terminal part of the split selectable marker and the N-terminal part of the split selectable marker may be the C-terminal part and the N-terminal part of a puromycin resistance gene (PuroR) split at amino acid position 119.

The C-terminal part of the split selectable marker and the N-terminal part of the split selectable marker may be the C-terminal part and the N-terminal part of a neomycin resistance gene (Neo^{R}) split at amino acid position 133. The C-terminal part of the split selectable marker and the N-terminal part of the split selectable marker may be the C-terminal part and the N-terminal part of a neomycin resistance gene (Neo^{R}) split at amino acid position 195.

The C-terminal part of the split selectable marker and the N-terminal part of the split selectable marker may be the C-terminal part and the N-terminal part of a gene encoding mScarlet fluorescent protein split at amino acid position 46. The C-terminal part of the split selectable marker and the N-terminal part of the split selectable marker may be the C-terminal part and the N-terminal part of a gene encoding mScarlet fluorescent protein split at amino acid position 48. The C-terminal part of the split selectable marker and the N-terminal part of the split selectable marker may be the C-terminal part and the N-terminal part of a gene encoding mScarlet fluorescent protein split at amino acid position 51. The C-terminal part of the split selectable marker and the N-terminal part of the split selectable marker may be the C-terminal part and the N-terminal part of a gene encoding mScarlet fluorescent protein split at amino acid position 75. The C-terminal part of the split selectable marker and the N-terminal part of the split selectable marker may be the C-terminal part and the N-terminal part of a gene encoding mScarlet fluorescent protein split at amino acid position 122. The C-terminal part of the split selectable marker and the N-terminal part of the split selectable marker may be the C-terminal part and the N-terminal part of a gene encoding mScarlet fluorescent protein split at amino acid position 140. The C-terminal part of the split selectable marker and the N-terminal part of the split selectable marker may be the C-terminal part and the N-terminal part of a gene encoding mScarlet fluorescent protein split at amino acid position 163.

The C-terminal part of the split selectable marker and the N-terminal part of the split selectable marker may be the C-terminal part and the N-terminal part of a luciferase protein split at amino acid position 437.

The C-terminal part of the split selectable marker and the N-terminal part of the split selectable marker may be the C-terminal part and the N-terminal part of a split glutamine synthetase protein.

In some preferred embodiments, the C-terminal part of the split selectable marker and the N-terminal part of the split selectable marker may be the C-terminal part and the N-terminal part of a blasticidin resistance gene (BsrR) split at amino acid position 102.

In some preferred embodiments, the C-terminal part of the split selectable marker comprises or consist of SEQ ID NO: 6 or a functional variant thereof. In some preferred embodiments, the N-terminal part of a split selectable marker comprises or consist of SEQ ID NO: 7 or a functional variant thereof. In some embodiments, the C-terminal part of the split selectable marker comprises or consist of SEQ ID NO: 6 or a functional variant thereof and the N-terminal part of a split selectable marker comprises or consist of SEQ ID NO: 7 or a functional variant thereof.

In some embodiments, the landing pad nucleic acid further comprises a third promoter operably linked to a second selectable marker. A second selectable marker may be used to select for integration of the landing pad nucleic acid into the genome of a cell. However, the presence of a second selectable marker is not absolutely necessary since if the landing pad nucleic acid is not integrated into the genome of interest and the delivery nucleic acid subsequently integrates into the landing pad nucleic acid, the resulting construct will not be replicated during cell division. In embodiments wherein the system does not comprise a second selectable marker, the landing pad nucleic acid may be delivered to cells via lentivirus, dilution cloning may be performed to isolate clones, clones may be retargeted with the delivery nucleic acid and finally selected with the split selectable marker (such as blasticidin). Any clones which survive this selection will carry the landing pad nucleic acid in the genome.

The second selectable marker may be any suitable marker gene which expresses a marker that is readily detectable, any and many such marker genes are well-known in the art. For example, the second selectable marker gene may be an antibiotic resistance gene, a gene encoding a fluorescent protein, a gene encoding glutamine synthetase or a gene encoding a luminescent protein. Suitably, the second selectable marker is different to the first selectable marker.

The second selectable marker may be a gene encoding a fluorescent protein. A fluorescent protein allows selection based on the presence or absence of a fluorescent protein (e.g. by FACs). The second selectable marker may be a gene encoding glutamine synthetase. Glutamine synthetase allows selection based on the ability of a cell to produce glutamine which is an essential amino acid required for cell survival. Some mammalian cells are not able to produce glutamine. For other cells an inhibitor of glutamine synthase (MSX) may be used. The second selectable marker may be a gene encoding a luminescent protein. A luminescent protein allows selection based on the presence or absence of a luminescent protein.

In some embodiments, the second selectable marker gene is an antibiotic resistance gene. An antibiotic resistance gene allows selection based on the ability of a cell to defeat an antibiotic. Antibiotic resistance gene is a particularly preferred second selectable marker gene as it allows for an efficient selection process as cells are simply grown in a substrate which includes the antibiotic for the respective antibiotic resistance gene.

The second selectable marker may be selected from the group consisting of: kanamycin resistance gene, spectinomycin resistance gene, streptomycin resistance gene, ampicillin resistance gene, carbenicillin resistance gene, bleomycin resistance gene, erythromycin resistance gene, polymyxin B resistance gene, tetracycline resistance gene, chloramphenicol resistance gene, hygromycin resistance gene, puromycin resistance gene, neomycin resistance gene and blasticidin resistance gene.

In some embodiments, the second selectable marker is hygromycin.

In some embodiments, the landing pad nucleic acid further comprises an IRES followed by a further selectable marker. In one embodiment, the IRES followed by a further selectable marker is located 3′ from the C-terminal part of a split selectable marker.

The further selectable marker may be any suitable marker gene which expresses a marker that is readily detectable, any and many such marker genes are well-known in the art. For example, the further selectable marker gene may be an antibiotic resistance gene, a gene encoding a fluorescent protein, a gene encoding glutamine synthetase or a gene encoding a luminescent protein. Suitably, the further selectable marker is different from the first selectable marker and the second selectable marker. The further selectable marker may be used to detect successful creation of a retargeted nucleic acid. The further selectable marker is an optional feature of the system according to the first aspect of the present invention and is not required for the system to function.

The further selectable marker may be a gene encoding glutamine synthetase. Glutamine synthetase allows selection based on the ability of a cell to produce glutamine which is an essential amino acid required for cell survival. Some mammalian cells are not able to produce glutamine. For other cells an inhibitor of glutamine synthase (MSX) may be used. The further selectable marker may be a gene encoding a luminescent protein. A luminescent protein allows selection based on the presence or absence of a luminescent protein. The further selectable marker gene may be an antibiotic resistance gene. An antibiotic resistance gene allows selection based on the ability of a cell to defeat an antibiotic. The further selectable marker may be selected from the group consisting of: kanamycin resistance gene, spectinomycin resistance gene, streptomycin resistance gene, ampicillin resistance gene, carbenicillin resistance gene, bleomycin resistance gene, erythromycin resistance gene, polymyxin B resistance gene, tetracycline resistance gene, chloramphenicol resistance gene, hygromycin resistance gene, puromycin resistance gene, neomycin resistance gene and blasticidin resistance gene.

In some embodiments, the further selectable marker is a fluorescent protein. A fluorescent protein allows selection based on the presence or absence of a fluorescent protein (e.g. by FACs). A fluorescent protein allows quantification of cells in which the landing pad nucleic acid has been integrated into the genome of interest and subsequently the delivery nucleic acid has been integrated into the landing pad nucleic acid, resulting in reconstruction of the selectable marker following translation of the parts of the split selectable marker. The quantification may be done via FACs.

The further selectable marker may be selected from the group consisting of: EBFP, ECFP, EGFP, YFP, mHoneydew, mBanana, mOrange, tdTomato,mTangerine, mStrawberry, mCherry,mGrape1, mRaspberry, mGrape2 and mPlum.

In some embodiments, the further selectable marker is EGFP. In some embodiments, the further selectable marker comprises or consists of SEQ ID NO: 9 or a functional variant thereof.

In a second aspect, there is provided a cell comprising the system according to the first aspect or part of the system according to the first aspect. The cell may be any cell. The cell may be any cell suitable for production of a stable cell line. The cell may be an animal cell, more preferably a mammalian cell. The cell may be a human cell. In some embodiments, the cell is a HEK293 cell. HEK293 is a well-known immortalised cell line originally derived from a fetus. In some embodiments, the cell is a CHO-K1 cell. CHO-K1 cells are derived from a subclone of the parental CHO cell line. CHO cell line was derived from a biopsy of an ovary of an adult, female Chinese hamster.

In a third aspect, there is provided a cell comprising a landing pad nucleic acid, wherein the landing pad nucleic acid comprises in the 5′ to 3′ direction: an expression stop signal, a first site-specific recombination site, a first transcriptional or translational split mechanism, a first split intein and split selectable marker combination. In some embodiments, the landing pad nucleic acid comprises in the 5′ to 3′ direction: an expression stop signal, a first site-specific recombination site, a first transcriptional or translational split mechanism, a C-terminal part of a split intein, and a C-terminal part of a split selectable marker. In some embodiments, the landing pad nucleic acid comprising in the 5′ to 3′ direction: an expression stop signal, a first site-specific recombination site, a first transcriptional or translational split mechanism, N-terminal part of the split selectable marker, and a N-terminal part of the split intein. The cell according to this aspect is suitable for generation of a stable cell line via delivery of a delivery nucleic acid comprising in the 5′ to 3′ direction: a second split intein and split selectable marker combination (for example, N-terminal part of the split selectable marker and a N-terminal part of the split intein or C-terminal part of a split intein and a C-terminal part of a split selectable marker), a second transcriptional or translational split mechanism and a second site-specific recombination site and an integrating enzyme or a nucleic acid encoding an integrating enzyme into the cell. The delivery nucleic acid may further comprise a first promoter operably linked to a gene of interest. The second split intein and split selectable marker combination may be operably linked to the second promoter or to the first promoter. In embodiments wherein the second split intein and split selectable marker combination is operably linked to the first promoter, the second split intein and split selectable marker combination is separated from the gene of interest via a third transcriptional or translational split mechanism, suitably by an IRES.

In some embodiments, the landing pad nucleic acid is stably integrated into the genome of the cell.

In some embodiments, the cell further comprises a delivery nucleic acid comprising in the 5′ to 3′ direction: a second split intein and split selectable marker combination (for example, N-terminal part of the split selectable marker and a N-terminal part of the split intein or C-terminal part of a split intein and a C-terminal part of a split selectable marker), a second transcriptional or translational split mechanism and a second site-specific recombination site. The delivery nucleic acid may further comprise a first promoter operably linked to a gene of interest. The second split intein and split selectable marker combination may be operably linked to the second promoter or to the first promoter. In embodiments wherein the second split intein and split selectable marker combination is operably linked to the first promoter, the second split intein and split selectable marker combination is separated from the gene of interest via a third transcriptional or translational split mechanism, suitably by an IRES.

In some embodiments, the cell further comprises an integrating enzyme, or a nucleic acid encoding an integrating enzyme. The integrating enzyme may be configured to catalyse site specific recombination between the first site-specific recombination site and the second site-specific recombination site.

In some embodiments, the cell further comprises:
a delivery nucleic acid comprising in the 5′ to 3′ direction: a second split intein and split selectable marker combination (for example, N-terminal part of the split selectable marker and a N-terminal part of the split intein or C-terminal part of a split intein and a C-terminal part of a split selectable marker), a second transcriptional or translational split mechanism and a second site-specific recombination site, wherein the delivery nucleic acid further comprises a first promoter operably linked to the gene of interest; and
an integrating enzyme, or a nucleic acid encoding an integrating enzyme, wherein the integrating enzyme is configured to catalyse site specific recombination between the first site-specific recombination site and the second site-specific recombination site.

In some embodiments, the second split intein and split selectable marker combination may be operably linked to the second promoter or to the first promoter. In embodiments wherein the second split intein and split selectable marker combination is operably linked to the first promoter, the second split intein and split selectable marker combination is separated from the gene of interest via a third transcriptional or translational split mechanism, suitably by an IRES.

In a fourth aspect, there is provided a method for selecting a cell comprising a gene of interest, the method comprising the following steps:
providing a cell with a landing pad nucleic acid comprising in the 5′ to 3′ direction: an expression stop signal, a first site-specific recombination site, a first transcriptional or translational split mechanism, a first split intein and split selectable marker combination (for example, C-terminal part of a split intein and a C-terminal part of a split selectable marker or N-terminal part of the split selectable marker and, a N-terminal part of the split intein);
providing the cell with (i) a delivery nucleic acid comprising in the 5′ to 3′ direction: a second split intein and split selectable marker combination (for example, N-terminal part of the split selectable marker and a N-terminal part of the split intein or C-terminal part of a split intein and a C-terminal part of a split selectable marker), a second transcriptional or translational split mechanism and a second site-specific recombination site, wherein the delivery nucleic acid further comprises a first promoter operably linked to the gene of interest and (ii) an integrating enzyme, or a nucleic acid encoding an integrating enzyme, wherein the integrating enzyme is configured to catalyse site specific recombination between the first site-specific recombination site and the second site-specific recombination site; and
selecting a cell which expresses the selectable marker gene.

In some embodiments, the second split intein and split selectable marker combination may be operably linked to the second promoter or to the first promoter. In embodiments wherein the second split intein and split selectable marker combination is operably linked to the first promoter, the second split intein and split selectable marker combination is separated from the gene of interest via a third transcriptional or translational split mechanism, suitably by an IRES.

In some preferred embodiments, the steps of the method are performed in the specified order.

The landing pad nucleic acid may be provided to the cell via any suitable method. By way of non-limiting example, the landing pad nucleic acid may be transfected, transduced, or conjugated into the cell. Suitably the landing pad nucleic acid may be provided to the cell via electroporation, microinjection, gene gun, impalefection, hydrostatic pressure, continuous infusion, sonication, calcium phosphate-based transfection, cationic polymers-based transfection, lipofection-based transfection, fugene-based transfection or viral delivery.

In some embodiments, the landing pad nucleic acid is provided into the cell via viral delivery. In some embodiments, the landing pad nucleic acid is provided into the cell via lentiviral delivery.

The delivery nucleic acid may be provided to the cell via any suitable method. By way of non-limiting example, the delivery nucleic acid may be transfected, transduced, or conjugated into the cell. Suitably the delivery nucleic acid may be provided to the cell via electroporation, microinjection, gene gun, impalefection, hydrostatic pressure, continuous infusion, sonication, calcium phosphate-based transfection, cationic polymers-based transfection, lipofection-based transfection, fugene-based transfection or viral delivery (preferably via a non-integrating virus such as Adenovirus and/or Sendai virus vectors).

In some embodiments, the delivery nucleic acid is provided into the cell via lipofection-based transfection.

In some embodiments, the method further comprise culturing the cell under conditions to express the integrating enzyme, the C-terminal part of a split intein and the C-terminal part of a split selectable marker and the N-terminal part of the split selectable marker and a N-terminal part of the split intein.

In some embodiments, following providing the cell with (i) the delivery nucleic acid and (ii) the integrating enzyme, or the nucleic acid encoding an integrating enzyme, the integrating enzyme catalyses integration of the delivery nucleic acid into the landing pad nucleic acid, thereby the selectable marker is reconstructed following translation of the parts of the split selectable marker prior to selecting the cell which expresses the selectable marker gene.

In some embodiments, the split selectable marker is re-joined to form a functional selectable marker when the gene of interest is successfully integrated.

In some embodiments, expression of the selectable marker gene indicates that the gene of interest has been integrated into the landing pad nucleic acid.

In a fifth aspect, there is provided a landing pad nucleic acid comprising in the 5′ to 3′ direction: an expression stop signal, a site-specific recombination site, a transcriptional or translational split mechanism, a part of a split intein, and a part of a split selectable marker.

In a sixth aspect, there is provided a delivery nucleic acid comprising in the 5′ to 3′ direction: a part of a split selectable marker, a part of a split intein, a transcriptional or translational split mechanism and a site-specific recombination site, wherein the delivery nucleic acid further comprises a first promoter operably linked to a gene of interest. In some embodiments, the part of the split selectable marker and the part of the split intein is operably linked to the second promoter or to the first promoter. In embodiments wherein the second split intein and split selectable marker combination is operably linked to the first promoter, the second split intein and split selectable marker combination is separated from the gene of interest via a third transcriptional or translational split mechanism, suitably by an IRES.

In a seventh aspect, there is provided a kit for providing and selecting a cell with a gene of interest, the kit comprising:
a landing pad nucleic acid comprising in the 5′ to 3′ direction: an expression stop signal, a first site-specific recombination site, a first transcriptional or translational split mechanism, a first split intein and split selectable marker combination (for example, C-terminal part of a split intein and a C-terminal part of a split selectable marker or N-terminal part of the split selectable marker and a N-terminal part of the split intein);
a delivery nucleic acid comprising in the 5′ to 3′ direction: a second split intein and split selectable marker combination (for example, N-terminal part of the split selectable marker and a N-terminal part of the split intein or C-terminal part of a split intein and a C-terminal part of a split selectable marker), a second transcriptional or translational split mechanism and a second site-specific recombination site, wherein the delivery nucleic acid further comprises a first promoter operably linked to a gene of interest; and
an integrating enzyme, or a nucleic acid encoding an integrating enzyme, wherein the integrating enzyme is configured to catalyse site specific recombination between the first site-specific recombination site and the second site-specific recombination site.

In some embodiments, the second split intein and split selectable marker combination is operably linked to the second promoter or to the first promoter. In embodiments wherein the second split intein and split selectable marker combination is operably linked to the first promoter, the second split intein and split selectable marker combination is separated from the gene of interest via a third transcriptional or translational split mechanism, suitably by an IRES.

The kit may be suitable for providing a cell with a gene of interest. The kit may be suitable for selecting cells in which a gene of interest has been stably integrated. The kit may be suitable for providing a cell with a gene of interest and selecting cells in which a gene of interest has been stably integrated.

In some embodiments, the kit further comprises packaging and instructions for use. Suitably the instructions are for use in performing the methods of the invention.

In an eighth aspect, there is provided a method of generating a cell according to the third aspect, the method comprising the following steps:
providing a cell with a landing pad nucleic acid comprising in the 5′ to 3′ direction: an expression stop signal, a first site-specific recombination site, a first transcriptional or translational split mechanism, a first split intein and split selectable marker combination (for example, C-terminal part of a split intein and a C-terminal part of a split selectable marker and N-terminal part of the split selectable marker and a N-terminal part of the split intein).

Suitably in any of the fifth, sixth, seventh or eighth aspects, the landing pad nucleic acid and delivery nucleic acid may comprise any of the features as defined herein in relation to a landing pad nucleic acid and delivery nucleic acid. Suitably in the eighth aspect, the cell and means of providing the cell with the nucleic acid may comprise any of the features as defined herein in relation to a cell and means of providing the cell with a nucleic acid.

### BRIEF DESCRIPTION OF THE FIGURES

FIG. 1A shows a schematic representation of the parts of the targeted integration and selection system according to one preferred embodiment of the present invention before integration. The landing pad nucleic acid (herein called pPlatform-EGFP) comprises in 5′ to 3′ direction: 3 stop codons, attB site-specific recombination site, furin-T2A linker, C terminal Npu DnaE split intein (Int_{C}) attached to the C-terminal part of the split blasticidin selectable marker (r^{R}). For convenience, the construct further comprises an internal ribosome entry site and GFP which allows visualisation and quantification of cells in which two successful integration events have resulted in the construct shown in FIG. 1B. Similarly, the construct also comprises a promoter operably linked to hygromycin to make selection of cells in which pPlatform-EGFP has been integrated into the genome easier. The delivery nucleic acid (pDeliver-mCherry) comprises in 5′ to 3′ direction: a constitutive promoter operably linked to N-terminal part of the split blasticidin selectable marker (Bs) attached to N terminal Npu DnaE split intein (Int_{N}), furin-T2A linker and attP site-specific recombination site. The construct further comprises a promoter operably linked to mCherry which allows visualisation and quantification of cells in which pDeliver-mCherry is present.

FIG. 1B shows a schematic representation of pDeliver-mCherry integrated into the pPlatform-EGFP construct which is in turn integrated into a genome. The post recombination coding sequence is cleaved into three separate proteins by the action of the T2A and furin cleavage motifs leaving the NpuDnaE split intein elements (Int_{C} and Int_{N}) free to splice together the N-terminal and C-terminal portions of the Bsr^{R} protein (Bs and r^{R}).

FIG. 2A shows a schematic representation of the pDeliver mCherry plasmid (retargeting plasmid)

FIG. 2B shows a schematic representation of the pIntegrase plasmid (BXB1 expression plasmid)

FIG. 2C shows a schematic representation of the pPlatform EGFP (landing pad) lentivirus

FIG. 2D shows a schematic representation of the integration of phage DNA into the host bacterial DNA by means of recombination between attachment sites (attP and attB) catalysed by phage integrase enzyme such as BXB1.

FIG. 3A shows flow cytometry data from CHO-K1 pPlatform EGFP (landing pad) cells retargeted with pDeliver mCherry plasmid (retargeting plasmid). pPlatform cells are CHO-K1 cells which were transduced at low MOI with the pPlatform EGFP construct and selected using hygromycin. Retargeted cells are pPlatform cells which were co-transfected with the mCherry pDeliver retargeting and BXB1 expression plasmids and grown in blasticidin selection media. Both pPlatform cells and retargeted cells were analysed for mCherry and EGFP expression by flow cytometry.

FIG. 3B shows flow cytometry data from HEK293 pPlatform EGFP (landing pad) cells retargeted with pDeliver mCherry plasmid (retargeting plasmid). pPlatform cells are HEK293 cells which were transduced at low MOI with the pPlatform EGFP construct and selected using hygromycin. Retargeted cells are pPlatform cells which were co-transfected with the mCherry pDeliver retargeting and BXB1 expression plasmids and grown in blasticidin selection media. Both pPlatform cells and retargeted cells were analysed for mCherry and EGFP expression by flow cytometry.

FIG. 4A shows a schematic representation of parts of the targeted integration and selection system according to another embodiment of the present invention before integration of the delivery nucleic acid into a cell line in which the landing pad nucleic acid is integrated. The landing pad nucleic acid comprises in 5′ to 3′ direction: 3 stop codons, attB site-specific recombination site, furin-T2A linker, N-terminal part of the split blasticidin selectable marker (Bs) attached to N terminal Npu DnaE split intein (Int_{N}). For convenience, the construct further comprises an internal ribosome entry site and GFP which allows visualisation and quantification of cells in which two successful integration events have resulted in the construct shown in FIG. 4B. Similarly, the construct also comprises a promoter operably linked to hygromycin to make selection of cells in which pPlatform-EGFP has been integrated into the genome easier. The delivery nucleic acid (pDeliver-mCherry) comprises in 5′ to 3′ direction: a constitutive promoter operably linked to C terminal Npu DnaE split intein (Int_{C}) attached to the C-terminal part of the split blasticidin selectable marker (r^{R}), furin-T2A linker and attP site-specific recombination site. The construct further comprises a promoter operably linked to mCherry which allows visualisation and quantification of cells in which pDeliver-mCherry is present.

FIG. 4B shows a schematic representation of pDeliver-mCherry integrated into the pPlatform-EGFP construct which is in turn integrated into a genome. The post recombination coding sequence is cleaved into three separate proteins by the action of the T2A and furin cleavage motifs leaving the NpuDnaE split intein elements (Int_{C} and Int_{N}) free to splice together the N-terminal and C-terminal portions of the Bsr^{R} protein (Bs and r^{R}).

FIG. 5A shows a schematic representation of parts of the targeted integration and selection system according to yet another embodiment of the present invention before integration of the delivery nucleic acid into a cell line in which the landing pad nucleic acid is integrated. The landing pad nucleic acid (herein called pPlatform-EGFP) comprises in 5′ to 3′ direction: 3 stop codons, attB site-specific recombination site, furin-T2A linker, C terminal Npu DnaE split intein (Int_{C}) attached to the C-terminal part of the split blasticidin selectable marker (r^{R}). For convenience, the construct further comprises an internal ribosome entry site and GFP which allows visualisation and quantification of cells in which two successful integration events have resulted in the construct shown in FIG. 5B. Similarly, the construct also comprises a promoter operably linked to hygromycin to make selection of cells in which pPlatform-EGFP has been integrated into the genome easier. The delivery nucleic acid (pDeliver-mCherry) comprises in 5′ to 3′ direction: a constitutive promoter operably linked to mCherry (which allows visualisation and quantification of cells in which pDeliver-mCherry is present), IRES, N-terminal part of the split blasticidin selectable marker (Bs) attached to N terminal Npu DnaE split intein (Int_{N}), furin-T2A linker and attP site-specific recombination site.

FIG. 5B shows a schematic representation of pDeliver-mCherry integrated into the pPlatform-EGFP construct which is in turn integrated into a genome. The post recombination coding sequence is cleaved into three separate proteins by the action of the T2A and furin cleavage motifs leaving the NpuDnaE split intein elements (Int_{C} and Int_{N}) free to splice together the N-terminal and C-terminal portions of the Bsr^{R} protein (Bs and r^{R}).

FIG. 6A shows flow cytometry data from CHO-K1 pPlatform EGFP (landing pad) cells retargeted with pDeliver CD19 plasmid (retargeting plasmid). pPlatform cells are CHO-K1 cells which were transduced at low MOI with the pPlatform EGFP construct and selected using hygromycin. Retargeted cells are pPlatform cells which were co-transfected with the pDeliver-CD19 retargeting and BXB1 expression plasmids and grown in blasticidin selection media. Both pPlatform cells and retargeted cells were analysed for CD19 and EGFP expression by flow cytometry.

FIG. 6B shows shows flow cytometry data from CHO-K1 pPlatform EGFP (landing pad) cells retargeted with pDeliver FOLR1A plasmid (retargeting plasmid). pPlatform cells are CHO-K1 cells which were transduced at low MOI with the pPlatform EGFP construct and selected using hygromycin. Retargeted cells are pPlatform cells which were co-transfected with the pDeliver-FOLR1A retargeting and BXB1 expression plasmids and grown in blasticidin selection media. Both pPlatform cells and retargeted cells were analysed for FOLR1A and EGFP expression by flow cytometry.

### DETAILED DESCRIPTION OF EMBODIMENTS OF THE INVENTION AND EXAMPLES

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. The invention will now be further described with reference to the following headed sections. Any of the features described in any of the sections may be applied to any of the aspects of the invention in any workable combination.

### System

The landing pad nucleic acid is for integration in a genome of a cell. The landing pad nucleic acid is configured to integrate into a genome of a cell.

The delivery nucleic acid is for integration of a gene of interest into the landing pad nucleic acid. The delivery nucleic acid is configured to integrate a gene of interest into the landing pad nucleic acid.

In some embodiments, the landing pad nucleic acid comprises or consists of SEQ ID NO: 10 or a functional variant thereof. In some embodiments, the landing pad nucleic acid comprises or consists of SEQ ID NO: 15 or a functional variant thereof.

In some embodiments, the delivery nucleic acid comprises or consists of SEQ ID NO: 14 or a functional variant thereof.

### Integrating Enzyme

The integrating enzyme may be configured to catalyse site specific recombination between the first site-specific recombination site and the second site-specific recombination site. In some embodiments, the integrating enzyme may be provided as a nucleic acid encoding an integrating enzyme. In some embodiments, the integrating enzyme may be provided as an integrating enzyme protein.

In some embodiments, the integrating enzyme is a unidirectional serine integrase. Unidirectional integration may be particularly preferred in generating a stable cell line expressing a gene of interest as it cannot be reversed by subsequent integration events. Without wishing to be bound to theory, the unidirectional serine integrase binds to the attachment sites, attP and attB, which are then brought together by protein-protein interactions to form a synaptic tetramer. Concerted double strand breaks are formed in both of the DNA substrates prior to subunit rotation and recombination.

In some preferred embodiments, the integrating enzyme is BxB1. BxB1 serine recombinase catalyses highly efficient unidirectional recombination between short heterologous attP and attB target sites resulting in the integration or deletion of DNA depending on the orientation and location of attP and attB sites. Recombination between attP and attB sites creates attL and attR sites, which are hybrid sites between attP and attB sites. BXB1 has been found to be functional in different mammalian cells and is the most efficient and most accurate out of the unidirectional serine integrases. BXB1 has been found to be most efficient and most accurate out of the unidirectional serine integrases in both mouse and human. In some embodiments, the nucleic acid encoding the integrating enzyme comprises or consists of SEQ ID NO: 13 or a functional variant thereof. In some embodiments, the nucleic acid encoding the integrating enzyme is operably linked to a fourth promoter.

The fourth promoter may be any promoter which is suitable for expression of a gene of interest in any cells. The fourth promoter may be any promoter which is suitable for expression of a gene of interest in mammalian cells. The fourth promoter may be a constitutive promoter, that is a promoter which results in continuously expression of the gene of interest. The fourth promoter may be an inducible promoter, that is a promoter which results in expression of the gene of interest when the respective inducer is added. In some embodiments, the fourth promoter is EF-1a promoter. In some embodiments the fourth promoter comprises or consists of SEQ ID NO: 16 or a functional variant thereof.

The integrating enzyme may be a recombinase. The integrating enzyme may be a tyrosine recombinase. Suitably, the tyrosine recombinase may be Cre recombinase. Suitably, the tyrosine recombinase may be Flp. Suitably, the integrating enzyme may be a CRISPR recombinase.

### First Site-Specific Recombination Site and Second Site-Specific Recombination Site

In some embodiments, the first site-specific recombination site is BXB1 attB. In some embodiments, the first site-specific recombination site comprises or consists of SEQ ID NO: 1 or a functional variant thereof. In some embodiments, the second site-specific recombination site is Bxb attP. In some embodiments, the second site-specific recombination site comprises or consists of SEQ ID NO: 2 or a functional variant thereof. In some embodiments, the first and second site-specific recombination sites may be reversed.

Suitably, when the integrating enzyme is Cre recombinase, the first site-specific recombination site is LoxP. Suitably, when the integrating enzyme is Cre recombinase, the second site-specific recombination site is LoxP. Suitably, when the integrating enzyme is Flp recombinase, the first site-specific recombination site is FRT. Suitably, when the integrating enzyme is Flp recombinase, the second site-specific recombination site is FRT.

### C-Terminal Part of a Split Intein and N-Terminal Part of a Split Intein

In some embodiments, the split marker gene is split into two segments—a C-terminal part of a split selectable marker and a N-terminal part of the split selectable marker. In some embodiments, the C-terminal part of a split intein is linked to a C-terminal part of a split selectable marker. In some embodiments, the C-terminal part of a split intein is fused to a C-terminal part of a split selectable marker. In some embodiments, the N-terminal part of the split selectable marker is linked to a N-terminal part of the split intein. In some embodiments, the N-terminal part of the split selectable marker is fused to a N-terminal part of the split intein.

The C-terminal part of a split selectable marker and the N-terminal part of the split selectable marker can be re-joined by protein trans splicing. During protein splicing, an intervening sequence (C-terminal part of a split intein and/or N-terminal part of a split intein) auto-catalytically excises itself from the precursor protein, and concomitantly ligates the two flanking sequences (C-terminal part of a split selectable marker and/or N-terminal part of a split selectable marker) with a peptide bond. The two flanking sequences are also called exteins. A split intein can catalyze protein ligation in trans, ligating the two exteins in the two polypeptide chains into one polypeptide chain.

In some embodiments, the C-terminal part of the split intein is the C-terminal part of the NpuDnaE intein. In some embodiments, the C-terminal part of the split intein comprises or consists of SEQ ID NO: 4 or a functional variant thereof. In some embodiments, the N-terminal part of the split intein is the N-terminal part of the NpuDnaE intein. In some embodiments, the N-terminal part of the split intein comprises or consists of SEQ ID NO: 5 or a functional variant thereof. In some embodiments, the C-terminal part of the split intein comprises or consists of SEQ ID NO: 4 and the N-terminal part of the split intein comprises or consists of SEQ ID NO: 5 or a functional variant thereof.

### Transcriptional or Translational Split Mechanism

The first transcriptional or translational split mechanism may be an internal ribosome entry site (IRES) or a 2A peptide. Internal ribosome entry site is an RNA element that allows for translation initiation in cap-independent manner. 2A peptides are approximately 20 amino acids long and self-cleavage occurs between the last 2 amino acids, glycine and proline. Choosing the first transcriptional or translational split mechanism allows control over the relative expression of the nucleic acid downstream and upstream of the second transcriptional or translational split mechanism. For example, in embodiments wherein the first transcriptional or translational split mechanism is IRES, it is expected that there will be less transcription of the downstream nucleic acid compared to the upstream nucleic acid. For example, in embodiments wherein the first transcriptional or translational split mechanism is 2A peptide, 1:1 ratio of the upstream and downstream nucleic acids is expected. In some embodiments, 2A peptide may be preferred as 1:1 ratio of the N-terminal part and C-terminal part of the split selectable markers may ensure that the system works more efficiently.

The mechanism of action of IRES and 2A peptides for co-expression of multiple genes in one transcript is also different. In embodiments wherein the first transcriptional or translational split mechanism is IRES, the gene directly downstream of promoter is translated by canonical cap-dependent mechanism while the gene downstream of IRES is translated by cap-independent mechanism and has lower translation efficiency. In embodiments wherein the first transcriptional or translational split mechanism is 2A peptide, the 2A linked genes are translated in one open reading frame and self-cleavage occurs post-translationally to give equal amounts of co-expressed protein.

The first transcriptional or translational split mechanism may be a self-cleaving peptide such as a 2A peptide selected from the group consisting of: F2A, P2A, E2A, T2A, GF2A, GP2A, GE2A and GT2A. In some embodiments, the first transcriptional or translational split mechanism is a T2A peptide. T2A peptide has the highest efficiency out of the group of 2A peptides.

In some embodiments, the first transcriptional or translational split mechanism is a 2A peptide with a furin recognition site. The consensus sequence of a furin cleavage site or a furin recognition site is RXXR, wherein X is any amino acid. In some embodiments, the first transcriptional or translational split mechanism is a T2A peptide with a furin recognition site. In some embodiments, the first transcriptional or translational split mechanism comprises or consist of SEQ ID NO: 3 or SEQ ID NO: 22 or a functional variant thereof.

The second transcriptional or translational split mechanism may be an internal ribosome entry site (IRES) or a 2A peptide. Internal ribosome entry site is an RNA element that allows for translation initiation in cap-independent manner. 2A peptides are approximately 20 amino acids long and self-cleavage occurs between the last 2 amino acids, glycine and proline. Choosing the second transcriptional or translational split mechanism allows control over the relative expression of the nucleic acid downstream and upstream of the second transcriptional or translational split mechanism. For example, in embodiments wherein the second transcriptional or translational split mechanism is IRES, it is expected that there will be less transcription of the downstream nucleic acid compared to the upstream nucleic acid. For example, in embodiments wherein the second transcriptional or translational split mechanism is 2A peptide, 1:1 ratio of the upstream and downstream nucleic acids is expected. In some embodiments, 2A peptide may be preferred as 1:1 ratio of the N-terminal part and C-terminal part of the split selectable markers may ensure that the system works more efficiently.

The mechanism of action of IRES and 2A peptides for co-expression of multiple genes in one transcript is also different. In embodiments wherein the first transcriptional or translational split mechanism is IRES, the gene directly downstream of promoter is translated by canonical cap-dependent mechanism while the gene downstream of IRES is translated by cap-independent mechanism and has lower translation efficiency. In embodiments wherein the first transcriptional or translational split mechanism is 2A peptide, the 2A linked genes are translated in one open reading frame and self-cleavage occurs post-translationally to give equal amounts of co-expressed protein.

In some embodiments, the first transcriptional or translational split mechanism is a T2A peptide. T2A peptide has the highest efficiency out of the group of 2A peptides.

In some embodiments, the second transcriptional or translational split mechanism is a 2A peptide with a furin recognition site. In some embodiments, the second transcriptional or translational split mechanism is a T2A peptide with a furin recognition site. In some embodiments, the second transcriptional or translational split mechanism comprises or consist of SEQ ID NO: 3 or SEQ ID NO: 23 or a functional variant thereof.

In some embodiments, the first transcriptional or translational split mechanism is a 2A peptide with a furin recognition site and the second transcriptional or translational split mechanism is a 2A peptide with a furin recognition site. In some embodiments, the first transcriptional or translational split mechanism is a T2A peptide with a furin recognition site and the second transcriptional or translational split mechanism is a T2A peptide with a furin recognition site. In some embodiments, the first transcriptional or translational split mechanism comprises or consist of SEQ ID NO: 3 or SEQ ID NO: 22 or a functional variant thereof and the second transcriptional or translational split mechanism comprises or consist of SEQ ID NO: 3 or SEQ ID NO: 23 or a functional variant thereof.

### Split Selectable Marker

In some embodiments, the N-terminal part of the split selectable marker and the C-terminal part split selectable marker are configured to be re-joined to form a functional selectable marker.

In some embodiments, a gene encoding a selectable marker is split into two segments to provide the N-terminal split selectable marker and C-terminal split selectable marker. In some embodiments, a gene encoding a selectable marker is split into three segments to provide the N-terminal split selectable marker, the C-terminal split selectable marker and a further fragment. In some embodiments, a gene encoding a selectable marker is split into four segments to provide the N-terminal split selectable marker, the C-terminal split selectable marker and further two fragments. In some embodiments, a gene encoding a selectable marker is split into five segments to provide the N-terminal split selectable marker, the C-terminal split selectable marker and further three fragment.

In some embodiments, the split selectable marker is an antibiotic resistance gene. An antibiotic resistance gene allows selection based on the ability of a cell to defeat an antibiotic. Antibiotic resistance gene is a particularly preferred split selectable marker as it allows for an efficient selection process as cells are simply grown in a substrate which includes the antibiotic for the respective antibiotic resistance gene.

Suitably the split selectable marker may be split at any position in the amino acid sequence thereof, suitably to create a C-terminal part and an N-terminal part.

In some embodiments, the C-terminal part of the split selectable marker and the N-terminal part of the split selectable marker may be the C-terminal part and the N-terminal part of a blasticidin resistance gene. In one embodiment, the blasticidin resistance gene (BsrR) split at amino acid position 102.

In some embodiments, the C-terminal part of the split selectable marker comprises or consist of SEQ ID NO: 6 or a functional variant thereof. In some embodiments, the N-terminal part of a split selectable marker comprises or consist of SEQ ID NO: 7 or a functional variant thereof. In some embodiments, the C-terminal part of the split selectable marker comprises or consist of SEQ ID NO: 6 or a functional variant thereof and the N-terminal part of a split selectable marker comprises or consist of SEQ ID NO: 7 or a functional variant thereof.

In some embodiments, the second split intein and split selectable marker combination is operably linked to the second promoter.

In some embodiments, the N-terminal part of a split selectable marker is operably linked to a second promoter. The second promoter may be any promoter which is suitable for expression of a gene of interest in any cells. The second promoter may be any promoter which is suitable for expression of a gene of interest in mammalian cells. The second promoter may be a constitutive promoter, that is a promoter which results in continuously expression of the gene of interest. The second promoter may be an inducible promoter, that is a promoter which results in expression of the gene of interest when the respective inducer is added. In some embodiments, the second promoter is CMV enhancer and promoter. In some embodiments, the second promoter comprises or consists of SEQ ID NO: 18 or a functional variant thereof. In some embodiments, the second promoter is SV40. In some embodiments, the second promoter comprises or consists of SEQ ID NO: 24 or a functional variant thereof. In some embodiments, the second promoter is PGK. In some embodiments, the second promoter comprises or consists of SEQ ID NO: 17 or a functional variant thereof.

In some embodiments, the second split intein and split selectable marker combination gene is operably linked to the first promoter. The first promoter may be any promoter which is suitable for expression of a gene of interest in mammalian cells. In some preferred embodiments, the first promoter is a constitutive promoter. In some embodiments, the first promoter is an inducible promoter, that is a promoter which results in expression of the gene of interest when the respective inducer is added. In some embodiments, the delivery nucleic acid comprises in the 5′ to 3′ direction: the first promoter, IRES and the second split intein and split selectable marker combination.

### Second Selectable Marker

The second selectable marker may be used for selection of cells in which the landing pad nucleic acid has been stably integrated. The second selectable marker may be used for selection of cells in which the landing pad nucleic acid has been stably integrated into the genome.

In some embodiments, the landing pad nucleic acid further comprises a third promoter operably linked to a second selectable marker. The third promoter may be any promoter which is suitable for expression of a gene of interest in any cells. The third promoter may be any promoter which is suitable for expression of a gene of interest in mammalian cells. The third promoter may be a constitutive promoter, that is a promoter which results in continuously expression of the gene of interest. The third promoter may be an inducible promoter, that is a promoter which results in expression of the gene of interest when the respective inducer is added. In some embodiments, the third promoter is PGK promoter. In some embodiments, the third promoter comprises or consists of SEQ ID NO: 17 or a functional variant thereof.

A second selectable marker may be used to select for integration of the landing pad nucleic acid into the genome of a cell. However, the presence of a second selectable marker is not necessary since if the landing pad nucleic acid is not integrated into the genome of interest and the delivery nucleic acid subsequently integrates into the landing pad nucleic acid, the resulting construct will not be replicated during cell division.

In some embodiments, the second selectable marker gene is an antibiotic resistance gene. An antibiotic resistance gene allows selection based on the ability of a cell to defeat an antibiotic. Antibiotic resistance gene is a particularly preferred second selectable marker gene as it allows for an efficient selection process as cells are simply grown in a substrate which includes the antibiotic for the respective antibiotic resistance gene. In some embodiments, the second selectable marker is hygromycin.

### An Expression Stop Signal

In some embodiments, the expression stop signal prevents any basal expression of the C-terminal split selectable marker in the landing pad nucleic acid. In the absence of the expression stop signal, some basal expression is possible even if there is no upstream promoter which is operably linked to the C-terminal split selectable marker in the landing pad nucleic acid. In the retargeted construct, the expression stop signal is positioned upstream of the first promoter operably linked to a gene of interest and no longer prevents the expression of the C-terminal split selectable marker.

In some preferred embodiments, the expression stop signal is one or more stop codons. The one or more stop codons may be, for example, 2, 3, 4, 5, 6, 7, 8, 9, or 10 stop codons. In some embodiments, the one or more stop codons are two sets of three stop codons. In some embodiments, the one or more stop codons comprise or consist of SEQ ID NO: 19 or a functional variant thereof. In some embodiments, the one or more stop codons comprise or consist of SEQ ID NO: 20 or a functional variant thereof.

### Gene of Interest

The gene of interest may encode for a protein of interest. The protein of interest may be selected from the group consisting of: antibodies (such as monoclonal antibodies), FC fusion proteins, anticoagulants, blood factors, enzymes, growth factors, hormones, interferons, interleukins, thrombolytics, Fc receptors, T cell receptors, cell surface receptors, and tumor-associated antigens (TAAs). In some embodiments, the protein of interest may be a monoclonal antibody.

In some embodiments, the gene of interest is operably liked to a first promoter. The first promoter may be any promoter which is suitable for expression of a gene of interest in any cells. The first promoter may be any promoter which is suitable for expression of a gene of interest in mammalian cells. The first promoter may be a constitutive promoter, that is a promoter which results in continuously expression of the gene of interest. The first promoter may be an inducible promoter, that is a promoter which results in expression of the gene of interest when the respective inducer is added. In some embodiments, the first promoter is EF-1a. In some embodiments, the first promoter comprises or consists of SEQ ID NO: 16 or a functional variant thereof.

The gene of interest operably liked to a first promoter may be positioned 5′ or 3′ with respect to the second split intein and split selectable marker combination (for example, the N-terminal part of the split selectable marker and the N-terminal part of the split intein or C-terminal part of a split intein, and a C-terminal part of a split selectable marker), the second transcriptional or translational split mechanism and the second site-specific recombination site in the delivery nucleic acid. The gene of interest operably liked to a first promoter may be positioned on either strand of a double stranded delivery nucleic acid. In embodiments wherein the second split intein and split selectable marker combination is operably linked to the first promoter, the gene of interest operably liked to a first promoter is preferably positioned 5′ with respect to the second split intein and split selectable marker combination, the second transcriptional or translational split mechanism and the second site-specific recombination site in the delivery nucleic acid.

In some embodiments, the gene of interest may be mCherry. In some embodiments, the gene of interest comprises or consists of SEQ ID NO: 12 or a functional variant thereof.

In some embodiments, the gene of interest may be FOLR1A. In some embodiments the gene of interest comprises or consists of SEQ ID NO: 25 or a functional variant thereof.

In some embodiments, the gene of interest may be CD19. In some embodiments the gene of interest comprises or consists of SEQ ID NO: 26 or a functional variant thereof.

### Definitions

As used herein, “gene of interest” refers to a nucleic acid sequence encoding a product of interest. The product of interest may be a protein of interest, e.g. a recombinant protein of interest.

“EFGP” refers to Enhanced Green Fluorescent Protein.

As used herein, “landing pad cell line” refers to a cell which comprises the landing pad nucleic acid. The landing pad nucleic acid may be stably integrated into the genome of the cell.

As used herein, “landing pad nucleic acid” refers to a nucleic acid which comprises in the 5′ to 3′ direction: an expression stop signal, a first site-specific recombination site, a first transcriptional or translational split mechanism, a C-terminal part of a split intein, and a C-terminal part of a split selectable marker.

As used herein, “stop codon” is a sequence of three nucleotides in DNA or messenger RNA which signal an end to protein synthesis.

As used herein, “delivery nucleic acid” refers to a nucleic acid which comprises in the 5′ to 3′ direction: a first promoter operably linked to a gene of interest, a second split intein and split selectable marker combination (for example, a N-terminal part of the split selectable marker, a N-terminal part of the split intein), a second transcriptional or translational split mechanism and a second site-specific recombination site. The second split intein and split selectable marker combination may be operably linked to the second promoter. The second split intein and split selectable marker combination may be operably linked to the first promoter.

As used herein, “site-specific recombination site” refers to short, specific nucleic acid sequence which are recognized and bound by site-specific recombinases. The skilled person will be familiar with various site-specific recombinases and corresponding site-specific recombination sites that may be used in embodiments of the invention. Non-limiting examples of such site-specific recombination sites include Bxb1 attB, Wβ attB, BL3 attB, R4 attB, A118 attB, TG1 attB, MR11 attB, φC370 attB, SPBc attB, TP901 attB, RV attB, FC1 attB, φK38 attB, φBT1 attB and C31 attB.

By “operably linked” as used herein, it is meant that the indicated elements are functionally related to each other, and are also generally physically related. Thus, the term “operably linked” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably linked with a nucleotide sequence if the promoter effects the transcription or expression of said nucleotide sequence. Those skilled in the art will appreciate that the control sequences (e.g., promoter) need not be contiguous with the nucleotide sequence to which it is operably linked, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence. DNA operably linked to a promoter is under transcriptional initiation regulation of the promoter or in functional combination therewith.

As used herein, the term “stably introduced” or “stably transformed” means that the nucleic acid sequence is stably incorporated into the genome of the cell, and thus the cell is stably transformed with the constructs. When a construct is stably transformed and therefore integrated into a cell, the integrated nucleic acid of the construct is capable of being inherited by the progeny thereof, more particularly, by the progeny of multiple successive generations.

As used herein, the term “transcriptional or translational split mechanism” refers to nucleic acid sequence or a protein sequence which allows two separate proteins to be created from a single nucleic acid. The first transcriptional or translational split mechanism may allow two separate proteins to be created from a single nucleic acid via a transcriptional mechanism. For example, the nucleic acids encoding each of the proteins may be operably linked to separate promoters resulting in two different transcripts. The first transcriptional or translational split mechanism may allow two separate proteins to be created from a single nucleic acid via a translational mechanism. The translational mechanism may be during translation or post-translational. For example, the transcriptional or translational split mechanism may be a 2A peptide.

As used herein, the term “split intein” refers to protein segments that are capable of enabling the ligation of flanking exteins into a new protein, in a process called protein splicing. In this process, the inteins are removed from a precursor protein with ligation of C-terminal and N-terminal external proteins (called exteins) on both sides. A “split intein” in the present context refers to an intein portion which can combine with a corresponding “split intein” to catalyse protein trans-splicing leading to the formation of a complete protein, e.g. selectable marker.

As used herein, the term “retargeted nucleic acid” refers to the resultant nucleic acid from the catalysation of site specific recombination between the first site-specific recombination site and the second site-specific recombination site by the integrating enzyme. The retargeted nucleic acid may comprise in the 5′ to 3′ direction:
an expression stop signal, attL, a first promoter operably linked to a gene of interest, a second promoter operably linked to a N-terminal part of the split selectable marker, a N-terminal part of the split intein, a second transcriptional or translational split mechanism, attR, a first transcriptional or translational split mechanism, a C-terminal part of a split intein, and a C-terminal part of a split selectable marker;
an expression stop signal, attL, a first promoter operably linked to a gene of interest, a second promoter operably linked to a C-terminal part of a split intein, and a C-terminal part of a split selectable marker, a first transcriptional or translational split mechanism, attR, a second transcriptional or translational split mechanism, a N-terminal part of the split selectable marker, a N-terminal part of the split intein; or
an expression stop signal, attL, a first promoter operably linked to a gene of interest, a third transcriptional or translational split mechanism, N-terminal part of the split selectable marker, a N-terminal part of the split intein, a second transcriptional or translational split mechanism, attR, a first transcriptional or translational split mechanism, a C-terminal part of a split intein, and a C-terminal part of a split selectable marker.

As used herein, the term “unidirectional serine integrase” refers to phage-encoded recombinase which promotes conservative recombination between two short DNA fragments—phage attachment site, attP and bacterial attachment site, attB. One of the features of these integrases is that their action is unidirectional, that is to say irreversible.

As used herein, the term “system” refers to a set of nucleic acids and optionally proteins working together as part or all of a mechanism for delivery of a gene of interest and selection of cells which comprise the gene of interest.

As used herein, the term “integrating enzyme” refers to an enzyme which is configured to integrate one nucleic acid (such as the delivery nucleic acid) into another nucleic acid (such as the landing pad nucleic acid). The integrating enzyme may be configured to catalyse site-specific recombination between the first site-specific recombination site and the second site-specific recombination site. As a result of this recombination, the delivery nucleic acid may be integrated into the landing pad nucleic acid. One example of an integrating enzyme is a unidirectional serine integrase. Further examples include Cre recombinase or Flp recombinase.

As used herein, the term “expression stop signal” refers to a sequence which prevents expression of the nucleic acids located 3′ with respect to the expression stop signal. In the landing pad nucleic acid, the expression stop signal prevents expression of the first site-specific recombination site, the first transcriptional or translational split mechanism, the C-terminal part of a split intein, and the C-terminal part of a split selectable markerfrom the landing pad nucleic acid. For example, the expression stop signal may be one or more stop codons.

As used herein, the term “functional variant” of a sequence (e.g. nucleic acid sequence or protein sequence) is a variant of a reference sequence that retains the ability to function in the same way as the reference sequence. For example, a functional variant of SEQ ID NO: 13 (BXB1 enzyme) may be a sequence which substantially retains enzyme activity. Alternative terms for such functional variants include “biological equivalents” or “equivalents”. Levels of sequence identity between a functional variant and the reference sequence can be an indicator of retained functionality. In some embodiments, the functional variant of a reference sequence comprises a sequence which has at least 70%, 80%, 90%, 95%, 96%, 97%, 98% or 99% identity to the reference sequence. For example, the functional variant of SEQ ID NO: 13 may comprise a sequence which has at least 70%, 80%, 90%, 95%, 96%, 97%, 98% or 99% identity to SEQ ID NO: 13.

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215:403-10), such as the “Blast 2 sequences” algorithm described by Tatusova and Madden 1999 (FEMS Microbiol Lett 174:247-250). Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10. The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn; Align Sequence Nucleotide BLAST) program may be employed using the default parameters. Nucleic acid sequences with even greater similarity to the reference sequences will show increasing percentage identity when assessed by this method. Typically, the percentage sequence identity is calculated over the entire length of the sequence. For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA RNA hybrid molecules, specifically including hnRNA, pre-mRNA, mRNA, cDNA, genomic DNA, amplification products, oligonucleotides, and synthetic (e.g., chemically synthesised) DNA, RNA or DNA RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

As used herein, “tumor-associated antigens” or “TAAs” refers to proteins or peptides directly or indirectly associated with the pathology of cancer (e.g. such as peptides presented by MHC class I or II molecules on the surface of tumour cells).

As used herein, “the second split intein and split selectable marker combination” and “the first split intein and split selectable marker combination” refer to a part of a split intein and a part of a split selectable marker. The part of a split intein may be 5′ or 3′ with respect to the part of a split selectable marker. The first split intein and split selectable marker combination is different to the second split intein and split selectable marker combination. In some embodiments, when the first split intein and split selectable marker combination comprises in the 5′ to 3′ direction N-terminal part of the split selectable marker and a N-terminal part of the split intein, the second split intein and split selectable marker combination comprises in the 5′ to 3′ direction C-terminal part of a split intein, and a C-terminal part of a split selectable marker. In some preferred embodiments, when the first split intein and split selectable marker combination comprises in the 5′ to 3′ direction C-terminal part of a split intein and a C-terminal part of a split selectable marker, the second split intein and split selectable marker combination comprises in the 5′ to 3′ direction N-terminal part of the split selectable marker and a N-terminal part of the split intein.

As used herein, “a part” refers to a portion of a nucleic acid sequence or amino acid sequence of a sufficient length to have the desired function. For example, a C-terminal “part” of a split selectable marker comprises sufficient amino acids to form a functional selectable marker when recombined with a N-terminal “part” of the split selectable marker. In another instance, a C-terminal “part” of a split intein comprises sufficient amino acids to form a functional intein when recombined with an N terminal part of the split intein.

### EXAMPLES

### Example 1—Sequences

BXB1 attB
(SEQ ID NO: 1)
TCGGCCGGCTTGTCGACGACGGCGGTCTCCGTCGTCAGGATCATCCGGGC
BXB1 attP (+ in frame stop codon)
(SEQ ID NO: 2)
GTCGTGGTTTGTCTGGTCAACCACCGCGGTCTCAGTGGTGTACGGTACAAACCCCGACGGTAA
Underlined is a stop codon and two nucleotides to ensure it is in frame
Furin-T2A linker (pDeliver)
(SEQ ID NO: 3)
GGCATCAGGAGGAAGAGGAGCGTGAGCCACGGAAGCGGAGGAAGCGGAGAGGGCA
GGGGAAGTCTTCTAACATGCGGGGACGTGGAGGAAAATCCCGGCCCC
Bold is furin cleavage motif
Underlined is Glycine-Serine-Glycine linker
Npu DnaE-C)
(SEQ ID NO: 4
ATGATCAAGATCGCCACCAGGAAGTACCTGGGCAAGCAGAACGTGTACGACATCGGCG
TGGAGAGGGACCACAACTTCGCCCTGAAGAACGGCTTCATCGCCAGCAAC
Npu DnaE-N
(SEQ ID NO: 5)
TGCCTGAGCTACGAGACCGAGATCCTGACCGTGGAGTACGGCCTGCTGCCCATCGGC
AAGATCGTGGAGAAGAGGATCGAGTGCACCGTGTACAGCGTGGACAACAACGGCAAC
ATCTACACCCAGCCCGTGGCCCAGTGGCACGACAGGGGCGAGCAGGAGGTGTTCGAG
TACTGCCTGGAGGACGGCAGCCTGATCAGGGCCACCAAGGACCACAAGTTCATGACC
GTGGACGGCCAGATGCTGCCCATCGACGAGATCTTCGAGAGGGAGCTGGACCTGATG
AGGGTGGACAACCTGCCCAAC
C-terminal Bsr^{R}
(SEQ ID NO: 6)
TGTAGGGAGTTGATTTCAGACTATGCACCAGATTGTTTTGTGTTAATAGAAATGAATGGC
AAGTTAGTCAAAACTACGATTGAAGAACTCATTCCACTCAAATATACCCGAAATTAA
N-terminal Bsr^{R}
(SEQ ID NO: 7)
ATGAAAACATTTAACATTTCTCAACAGGATCTAGAATTAGTAGAAGTAGCGACAGAGAAG
ATTACAATGCTTTATGAGGATAATAAACATCATGTGGGAGCGGCAATTCGTACGAAAAC
AGGAGAAATCATTTCGGCAGTACATATTGAAGCGTATATAGGACGAGTAACTGTTTGTG
CAGAAGCCATTGCGATTGGTAGTGCAGTTTCGAATGGACAAAAGGATTTTGACACGATT
GTAGCTGTTAGACACCCTTATTCTGACGAAGTAGATAGAAGTATTCGAGTGGTAAGTCC
TTGTGGTATG
Internal ribosome entry site (IRES)
(SEQ ID NO: 8)
CCCCTCTCCCTCCCCCCCCCCTAACGTTACTGGCCGAAGCCGCTTGGAATAAGGCCGG
TGTGCGTTTGTCTATATGTTATTTTCCACCATATTGCCGTCTTTTGGCAATGTGAGGGCC
CGGAAACCTGGCCCTGTCTTCTTGACGAGCATTCCTAGGGGTCTTTCCCCTCTCGCCA
AAGGAATGCAAGGTCTGTTGAATGTCGTGAAGGAAGCAGTTCCTCTGGAAGCTTCTTGA
AGACAAACAACGTCTGTAGCGACCCTTTGCAGGCAGCGGAACCCCCCACCTGGCGACA
GGTGCCTCTGCGGCCAAAAGCCACGTGTATAAGATACACCTGCAAAGGCGGCACAACC
CCAGTGCCACGTTGTGAGTTGGATAGTTGTGGAAAGAGTCAAATGGCTCTCCTCAAGC
GTATTCAACAAGGGGCTGAAGGATGCCCAGAAGGTACCCCATTGTATGGGATCTGATC
TGGGGCCTCGGTACACATGCTTTACATGTGTTTAGTCGAGGTTAAAAAAACGTCTAGGC
CCCCCGAACCACGGGGACGTGGTTTTCCTTTGAAAAACACGATGATAATATGGCCACAACC
EGFP
(SEQ ID NO: 9)
ATGGTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGATGC
CACCTACGGCAAGCTGACCCTGAAGTTCATCTGCACCACCGGCAAGCTGCCCGTGCCC
TGGCCCACCCTCGTGACCACCCTGACCTACGGCGTGCAGTGCTTCAGCCGCTACCCC
GACCACATGAAGCAGCACGACTTCTTCAAGTCCGCCATGCCCGAAGGCTACGTCCAGG
AGCGCACCATCTTCTTCAAGGACGACGGCAACTACAAGACCCGCGCCGAGGTGAAGTT
CGAGGGCGACACCCTGGTGAACCGCATCGAGCTGAAGGGCATCGACTTCAAGGAGGA
CGGCAACATCCTGGGGCACAAGCTGGAGTACAACTACAACAGCCACAACGTCTATATC
ATGGCCGACAAGCAGAAGAACGGCATCAAGGTGAACTTCAAGATCCGCCACAACATCG
AGGACGGCAGCGTGCAGCTCGCCGACCACTACCAGCAGAACACCCCCATCGGCGACG
GCCCCGTGCTGCTGCCCGACAACCACTACCTGAGCACCCAGTCCGCCCTGAGCAAAG
ACCCCAACGAGAAGCGCGATCACATGGTCCTGCTGGAGTTCGTGACCGCCGCCGGGA
TCACTCTCGGCATGGACGAGCTGTACAAGTAA
pPlatform landing pad-(BXB1 attB-Furin-T2A-Npu DnaE-BsrR (C-term)
(SEQ ID NO: 10)
TGATAATAGTCGGCCGGCTTGTCGACGACGGCGGTCTCCGTCGTCAGGATCATCCGG
GCGGCATCAGGAGGAAGAGGAGCGTGAGCCACGGAAGCGGAGGAAGCGGAGAGG
GCAGGGGAAGTCTTCTAACATGCGGGGACGTGGAGGAAAATCCCGGCCCCATGATC
AAGATCGCCACCAGGAAGTACCTGGGCAAGCAGAACGTGTACGACATCGGCGTGGAG
AGGGACCACAACTTCGCCCTGAAGAACGGCTTCATCGCCAGCAACTGTAGGGAGTTGA
TTTCAGACTATGCACCAGATTGTTTTGTGTTAATAGAAATGAATGGCAAGTTAGTCAA
AACTACGATTGAAGAACTCATTCCACTCAAATATACCCGAAATTAA
Underlined sequence indicates BXB1 attB
Bold sequence indicates Furin-T2A
Underlined and italicized sequence indicates Npu DnaE
Bold and italicized sequence indicates BsrR (C-term)
pDeliver retargeting sequence (N-terminal BsrR-Npu DnaE-N-Furin-T2A-BXB1 attP)
(SEQ ID NO: 11)
ATGAAAACATTTAACATTTCTCAACAGGATCTAGAATTAGTAGAAGTAGCGACAGAGA
AGATTACAATGCTTTATGAGGATAATAAACATCATGTGGGAGCGGCAATTCGTACGAA
AACAGGAGAAATCATTTCGGCAGTACATATTGAAGCGTATATAGGACGAGTAACTGTT
TGTGCAGAAGCCATTGCGATTGGTAGTGCAGTTTCGAATGGACAAAAGGATTTTGAC
ACGATTGTAGCTGTTAGACACCCTTATTCTGACGAAGTAGATAGAAGTATTCGAGTGG
TAAGTCCTTGTGGTATGTGCCTGAGCTACGAGACCGAGATCCTGACCGTGGAGTACGG
CCTGCTGCCCATCGGCAAGATCGTGGAGAAGAGGATCGAGTGCACCGTGTACAGCGT
GGACAACAACGGCAACATCTACACCCAGCCCGTGGCCCAGTGGCACGACAGGGGCGA
GCAGGAGGTGTTCGAGTACTGCCTGGAGGACGGCAGCCTGATCAGGGCCACCAAGGA
CCACAAGTTCATGACCGTGGACGGCCAGATGCTGCCCATCGACGAGATCTTCGAGAGG
GAGCTGGACCTGATGAGGGTGGACAACCTGCCCAACGGCATCAGGAGGAAGAGGAGC
GTGAGCCACGGAAGCGGAGAGGGCAGGGGAAGTCTTCTAACATGCGGGGACGTGGA
GGAAAATCCCGGCCCCGTCGTGGTTTGTCTGGTCAACCACCGCGGTCTCAGTGGTGTA
CGGTACAAACCCCGACGGTAA
Bold sequence indicates N-terminal Bsr^{R}
Underlined sequence indicates Npu DnaE-N
mCherry coding sequence
(SEQ ID NO: 12)
ATGGTGAGCAAGGGCGAGGAGGATAACATGGCCATCATCAAGGAGTTCATGCGCTTCA
AGGTGCACATGGAGGGCTCCGTGAACGGCCACGAGTTCGAGATCGAGGGCGAGGGC
GAGGGCCGCCCCTACGAGGGCACCCAGACCGCCAAGCTGAAGGTGACCAAGGGTGG
CCCCCTGCCCTTCGCCTGGGACATCCTGTCCCCTCAGTTCATGTACGGCTCCAAGGCC
TACGTGAAGCACCCCGCCGACATCCCCGACTACTTGAAGCTGTCCTTCCCCGAGGGCT
TCAAGTGGGAGCGCGTGATGAACTTCGAGGACGGCGGCGTGGTGACCGTGACCCAGG
ACTCCTCCCTGCAGGACGGCGAGTTCATCTACAAGGTGAAGCTGCGCGGCACCAACTT
CCCCTCCGACGGCCCCGTAATGCAGAAGAAGACCATGGGCTGGGAGGCCTCCTCCGA
GCGGATGTACCCCGAGGACGGCGCCCTGAAGGGCGAGATCAAGCAGAGGCTGAAGCT
GAAGGACGGCGGCCACTACGACGCTGAGGTCAAGACCACCTACAAGGCCAAGAAGCC
CGTGCAGCTGCCCGGCGCCTACAACGTCAACATCAAGTTGGACATCACCTCCCACAAC
GAGGACTACACCATCGTGGAACAGTACGAACGCGCCGAGGGCCGCCACTCCACCGGC
GGCATGGACGAGCTGTACAAGTAA
BXB1 coding sequence
(SEQ ID NO: 13)
ATGAGGGCCCTGGTGGTGATCAGGCTGAGCAGGGTGACCGACGCCACCACCAGCCCC
GAGAGGCAGCTGGAGAGCTGCCAGCAGCTGTGCGCCCAGAGGGGCTGGGACGTGGT
GGGCGTGGCCGAGGACCTGGACGTGAGCGGCGCCGTGGACCCCTTCGACAGGAAGA
GGAGGCCCAACCTGGCCAGGTGGCTGGCCTTCGAGGAGCAGCCCTTCGACGTGATCG
TGGCCTACAGGGTGGACAGGCTGACCAGGAGCATCAGGCACCTGCAGCAGCTGGTGC
ACTGGGCCGAGGACCACAAGAAGCTGGTGGTGAGCGCCACCGAGGCCCACTTCGACA
CCACCACCCCCTTCGCCGCCGTGGTGATCGCCCTGATGGGCACCGTGGCCCAGATGG
AGCTGGAGGCCATCAAGGAGAGGAACAGGAGCGCCGCCCACTTCAACATCAGGGCCG
GCAAGTACAGGGGCAGCCTGCCCCCCTGGGGCTACCTGCCCACCAGGGTGGACGGC
GAGTGGAGGCTGGTGCCCGACCCCGTGCAGAGGGAGAGGATCCTGGAGGTGTACCAC
AGGGTGGTGGACAACCACGAGCCCCTGCACCTGGTGGCCCACGACCTGAACAGGAGG
GGCGTGCTGAGCCCCAAGGACTACTTCGCCCAGCTGCAGGGCAGGGAGCCCCAGGG
CAGGGAGTGGAGCGCCACCGCCCTGAAGAGGAGCATGATCAGCGAGGCCATGCTGG
GCTACGCCACCCTGAACGGCAAGACCGTGAGGGACGACGACGGCGCCCCCCTGGTGA
GGGCCGAGCCCATCCTGACCAGGGAGCAGCTGGAGGCCCTGAGGGCCGAGCTGGTG
AAGACCAGCAGGGCCAAGCCCGCCGTGAGCACCCCCAGCCTGCTGCTGAGGGTGCTG
TTCTGCGCCGTGTGCGGCGAGCCCGCCTACAAGTTCGCCGGCGGCGGCAGGAAGCAC
CCCAGGTACAGGTGCAGGAGCATGGGCTTCCCCAAGCACTGCGGCAACGGCACCGTG
GCCATGGCCGAGTGGGACGCCTTCTGCGAGGAGCAGGTGCTGGACCTGCTGGGCGA
CGCCGAGAGGCTGGAGAAGGTGTGGGTGGCCGGCAGCGACAGCGCCGTGGAGCTGG
CCGAGGTGAACGCCGAGCTGGTGGACCTGACCAGCCTGATCGGCAGCCCCGCCTACA
GGGCCGGCAGCCCCCAGAGGGAGGCCCTGGACGCCAGGATCGCCGCCCTGGCCGCC
AGGCAGGAGGAGCTGGAGGGCCTGGAGGCCAGGCCCAGCGGCTGGGAGTGGAGGG
AGACCGGCCAGAGGTTCGGCGACTGGTGGAGGGAGCAGGACACCGCCGCCAAGAAC
ACCTGGCTGAGGAGCATGAACGTGAGGCTGACCTTCGACGTGAGGGGCGGCCTGACC
AGGACCATCGACTTCGGCGACCTGCAGGAGTACGAGCAGCACCTGAGGCTGGGCAGC
GTGGTGGAGAGGCTGCACACCGGCATGAGCTAA
pDeliver mCherry construct
(SEQ ID NO: 14)
caactttgtatagaaaagttgggctccggtgcccgtcagtgggcagagcgcacatcgcccacagtccccgagaagttgggggg
aggggtcggcaattgaaccggtgcctagagaaggtggcgcggggtaaactgggaaagtgatgtcgtgtactggctccgccttttt
cccgaggggggggagaaccgtatataagtgcagtagtcgccgtgaacgttctttttcgcaacgggtttgccgccagaacacag
gtaagtgccgtgtgtggttcccgcgggcctggcctctttacgggttatggcccttgcgtgccttgaattacttccacctggctgcagta
cgtgattcttgatcccgagcttcgggttggaagtgggtgggagagttcgaggccttgcgcttaaggagccccttcgcctcgtgcttg
agttgaggcctggcctgggcgctggggccgccgcgtgcgaatctggtggcaccttcgcgcctgtctcgctgctttcgataagtctct
agccatttaaaatttttgatgacctgctgcgacgctttttttctggcaagatagtcttgtaaatgcgggccaagatctgcacactggtatt
tcggtttttggggccgcgggcggcgacggggcccgtgcgtcccagcgcacatgttcggcgaggcggggcctgcgagcgcggc
caccgagaatcggacgggggtagtctcaagctggccggcctgctctggtgcctggtctcgcgccgccgtgtatcgccccgccct
gggcggcaaggctggcccggtcggcaccagttgcgtgagcggaaagatggccgcttcccggccctgctgcagggagctcaa
aatggaggacgcggcgctcgggagagcggggggtgagtcacccacacaaaggaaaagggcctttccgtcctcagccgtcg
cttcatgtgactccacggagtaccgggcgccgtccaggcacctcgattagttctcgagcttttggagtacgtcgtctttaggttgggg
ggaggggttttatgcgatggagtttccccacactgagtgggtggagactgaagttaggccagcttggcacttgatgtaattctccttg
gaatttgccctttttgagtttggatcttggttcattctcaagcctcagacagtggttcaaagtttttttcttccatttcaggtgtcgtgacaagt
ttgtacaaaaaagcaggctgccaccatggtgagcaagggcgaggaggataacatggccatcatcaaggagttcatgcgcttca
aggtgcacatggagggctccgtgaacggccacgagttcgagatcgagggcgagggcgagggccgcccctacgagggcacc
cagaccgccaagctgaaggtgaccaagggtggccccctgcccttcgcctgggacatcctgtcccctcagttcatgtacggctcca
aggcctacgtgaagcaccccgccgacatccccgactacttgaagctgtccttccccgagggcttcaagtgggagcgcgtgatga
acttcgaggacggcggcgtggtgaccgtgacccaggactcctccctgcaggacggcgagttcatctacaaggtgaagctgcgc
ggcaccaacttcccctccgacggccccgtaatgcagaagaagaccatgggctgggaggcctcctccgagcggatgtaccccg
aggacggcgccctgaagggcgagatcaagcagaggctgaagctgaaggacggcggccactacgacgctgaggtcaagac
cacctacaaggccaagaagcccgtgcagctgcccggcgcctacaacgtcaacatcaagttggacatcacctcccacaacga
ggactacaccatcgtggaacagtacgaacgcgccgagggccgccactccaccggcggcatggacgagctgtacaagtaag
gccgcgactctagagtcggggcggccggccgcttcgagcagacatgataagatacattgatgagtttggacaaaccacaacta
gaatgcagtgaaaaaaatgctttatttgtgaaatttgtgatgctattgctttatttgtaaccattataagctgcaataaacaagttaaca
acaacaattgcattcattttatgtttcaggttcagggggaggtgtgggaggttttttaaagcaagtaaaacctctacaaatgtggtaa
aatcgataagcatccgtttgcgtattgggcgctcttccgctgatctgcgcagcaccatgttctacccgttacataacttacggtaaatg
gcccgcctggctgaccgcccaacgacccccgcccattgacgtcaataatgacgtatgttcccatagtaacgccaatagggacttt
ccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctacgtat
tagtcatcgctattaccatggtgatgcggttttggcagtacatcaatgggcgtggatagcggtttgactcacggggatttccaagtctc
caccccattgacgtcaatgggagtttgttttggcaccaaaatcaacgggactttccaaaatgtcgtaacaactccgccccattgac
gcaaatgggcggtaggcgtgtacggtgggaggtctatataagcagagctctctggctaactgtcgggatcaccgaattcaccggt
gccgccaccatgaaaacatttaacatttctcaacaggatctagaattagtagaagtagcgacagagaagattacaatgctttatg
aggataataaacatcatgtgggagcggcaattcgtacgaaaacaggagaaatcatttcggcagtacatattgaagcgtatatag
gacgagtaactgtttgtgcagaagccattgcgattggtagtgcagtttcgaatggacaaaaggattttgacacgattgtagctgtta
gacacccttattctgacgaagtagatagaagtattcgagtggtaagtccttgtggtatgtgcctgagctacgagaccgagatcctg
accgtggagtacggcctgctgcccatcggcaagatcgtggagaagaggatcgagtgcaccgtgtacagcgtggacaacaac
ggcaacatctacacccagcccgtggcccagtggcacgacaggggcgagcaggaggtgttcgagtactgcctggaggacggc
agcctgatcagggccaccaaggaccacaagttcatgaccgtggacggccagatgctgcccatcgacgagatcttcgagaggg
agctggacctgatgagggtggacaacctgcccaacggcatcaggaggaagaggagcgtgagccacggaagcggagagg
gcaggggaagtcttctaacatgcggggacgtggaggaaaatcccggccccgtcgtggtttgtctggtcaaccaccgcggtctca
gtggtgtacggtacaaaccccgacggtaaacccagctttcttgtacaaagtggtgatggccggccgcttcgagcagacatgata
agatacattgatgagtttggacaaaccacaactagaatgcagtgaaaaaaatgctttatttgtgaaatttgtgatgctattgctttattt
gtaaccattataagctgcaataaacaagttaacaacaacaattgcattcattttatgtttcaggttcagggggaggtgtgggaggttt
tttaaagcaagtaaaacctctacaaatgtggtagcggccgcggcgctcttccgcttcctcgctcactgactcgctgcgctcggtcgtt
cggctgcggcgagcggtatcagctcactcaaaggcggtaatacggttatccacagaatcaggggataacgcaggaaagaac
atgtgagcaaaaggccagcaaaaggccaggaaccgtaaaaaggccgcgttgctggcgtttttccataggctccgcccccctga
cgagcatcacaaaaatcgacgctcaagtcagaggtggcgaaacccgacaggactataaagataccaggcgtttccccctgga
agctccctcgtgcgctctcctgttccgaccctgccgcttaccggatacctgtccgcctttctctcttcgggaagcgtggcgctttctcat
agctcacgctgtaggtatctcagttcggtgtaggtcgttcgctccaagctgggctgtgtgcacgaaccccccgttcagcccgaccg
ctgcgccttatccggtaactatcgtcttgagtccaacccggtaagacacgacttatcgccactggcagcagccactggtaacagg
attagcagagcgaggtatgtaggcggtgctacagagttcttgaagtggtggcctaactacggctacactagaagaacagtatttg
gtatctgcgctctgctgaagccagttaccttcggaaaaagagttggtagctcttgatccggcaaacaaaccaccgctggtagcgg
tggtttttttgtttgcaagcagcagattacgcgcagaaaaaaaggatctcaagaagatcctttgatcttttctacggggtctgacgctc
agtggaacgaaaactcacgttaagggattttggtcatgagattatcaaaaaggatcttcacctagatccttttaaattaaaaatgaa
gttttaaatcaatctaaagtatatatgagtaaacttggtctgacagttaccaatgcttaatcagtgaggcacctatctcagcgatctgt
ctatttcgttcatccatagttgcctgactccccgtcgtgtagataactacgatacgggagggcttaccatctggccccagtgctgcaa
tgataccgcgagacccacgctcaccggctccagatttatcagcaataaaccagccagccggaagggccgagcgcagaagtg
gtcctgcaactttatccgcctccatccagtctattaattgttgccgggaagctagagtaagtagttcgccagttaatagtttgcgcaac
gttgttgccattgctacaggcatcgtggtgtcacgctcgtcgtttggtatggcttcattcagctccggttcccaacgatcaaggcgagtt
acatgatcccccatgttgtgcaaaaaagcggttagctccttcggtcctccgatcgttgtcagaagtaagttggccgcagtgttatcac
tcatggttatggcagcactgcataattctcttactgtcatgccatccgtaagatgcttttctgtgactggtgagtactcaaccaagtcatt
ctgagaatagtgtatgcggcgaccgagttgctcttgcccggcgtcaatacgggataataccgcgccacatagcagaactttaaa
agtgctcatcattggaaaacgttcttcggggcgaaaactctcaaggatcttaccgctgttgagatccagttcgatgtaacccactcgt
gcacccaactgatcttcagcatcttttactttcaccagcgtttctgggtgagcaaaaacaggaaggcaaaatgccgcaaaaaag
ggaataagggcgacacggaaatgttgaatactcatactcttcctttttcaatattattgaagcatttatcagggttattgtctcatgagc
ggatacatatttgaatgtatttagaaaaataaacaaataggggttccgcgcacatttccccgaaaagtgccacctgacgtctaag
aaaccattattatcatgacattaacctataaaaataggcgtatcacgaggccctttcgtcggcgcgccgcggccgc
mCherry coding sequence is underlined.
pPlatform EGFP landing pad
(SEQ ID NO: 15)
gggtctctctggttagaccagatctgagcctgggagctctctggctaactagggaacccactgcttaagcctcaataaagcttgcct
tgagtgcttcaagtagtgtgtgcccgtctgttgtgtgactctggtaactagagatccctcagacccttttagtcagtgtggaaaatctct
agcagtggcgcccgaacagggacttgaaagcgaaagggaaaccagaggagctctctcgacgcaggactcggcttgctgaa
gcgcgcacggcaagaggcgaggggcggcgactggtgagtacgccaaaaattttgactagcggaggctagaaggagagag
atgggtgcgagagcgtcagtattaagcgggggagaattagatcgcgatgggaaaaaattcggttaaggccagggggaaaga
aaaaatataaattaaaacatatagtatgggcaagcagggagctagaacgattcgcagttaatcctggcctgttagaaacatcag
aaggctgtagacaaatactgggacagctacaaccatcccttcagacaggatcagaagaacttagatcattatataatacagtag
caaccctctattgtgtgcatcaaaggatagagataaaagacaccaaggaagctttagacaagatagaggaagagcaaaaca
aaagtaagaccaccgcacagcaagcggccgctgatcttcagacctggaggaggagatatgagggacaattggagaagtga
attatataaatataaagtagtaaaaattgaaccattaggagtagcacccaccaaggcaaagagaagagtggtgcagagagaa
aaaagagcagtgggaataggagctttgttccttgggttcttgggagcagcaggaagcactatgggcgcagcgtcaatgacgctg
acggtacaggccagacaattattgtctggtatagtgcagcagcagaacaatttgctgagggctattgaggcgcaacagcatctgt
tgcaactcacagtctggggcatcaagcagctccaggcaagaatcctggctgtggaaagatacctaaaggatcaacagctcctg
gggatttggggttgctctggaaaactcatttgcaccactgctgtgccttggaatgctagttggagtaataaatctctggaacagatttg
gaatcacacgacctggatggagtgggacagagaaattaacaattacacaagcttaatacactccttaattgaagaatcgcaaa
accagcaagaaaagaatgaacaagaattattggaattagataaatgggcaagtttgtggaattggtttaacataacaaattggct
gtggtatataaaattattcataatgatagtaggaggcttggtaggtttaagaatagtttttgctgtactttctatagtgaatagagttagg
cagggatattcaccattatcgtttcagacccacctcccaaccccgaggggacccgacaggcccgaaggaatagaagaagaa
ggtggagagagagacagagacagatccattcgattagtgaacggatctcgacggtatcgctagcttttaaaagaaaaggggg
gattggggggtacagtgcaggggaaagaatagtagacataatagcaacagacatacaaactaaagaattacaaaaacaaat
tacaaaaattcaaaattttactagtgattatcggatcaactttgtatagaaaagttgccagatttgtgcatacacagtgactcatacttt
caccaatactttgcattttggataaatactagacaactttagaagtgaattatttatgaggttgtcttaaaattaaaaattacaaagtaa
taaatcacattgtaatgtattttgtgtgatacccagaggtttaaggcaacctattactcttatgctcctgaagtccacaattcacagtcct
gaactataatcttatctttgtgattgctgagcaaatttgcagtataatttcagtgcttttaaattttgtcctgcttactattttccttttttatttggg
tttgatatgcgtgcacagaatggggcttctattaaaatattccatggcttacatttttaatattttgttctcttaatatgttcaaagctactca
acttttattcccgaaaaatgtttactttaattattctaatttcttacataaagcattgaggtgctaacaattatatactatgtacaagatggc
agactaaatcatatcataccatcaagtagaaacctggagtttggtgaactttgagttgtttatatgtctctcctttattgtcttctcaaaac
ctgtgattctgaagtcaaagggacacagctgtcacatgaaaagtgatcacttatcacctgtatgcataaaacaccttaccaagca
gctaagaggagtaactcctagccactttgagaaacgtttttgaataaacagagcaaggctcttccccattctcccagagatatagc
ataaaactgagcgcatttttataaaacaaaaaaggaggaatgtgtggtttgatggccagaccctgaatttgtgttcagcatctgctttt
ccatattatagatgggtaccagtgattctgagccatgtctatttctcctgacttttcctctgttttcccacgcttgctgatatttacagccgtg
gtcatcacaatcacctttgttcctttcttccttcctccaactctgcattaaattccaggaacttgctttctgtgaagtctaggggcgggact
ctggggttcgaaatgaccgaccaagcgacgcccaacctgccatcagtggtttgtctggtcaaccaccgcggtctcagtggtgtac
ggtacaaacccacaagtttgtacaaaaaagcaggctgccacctgataatagctgataatagtcggccggcttgtcgacgacgg
cggtctccgtcgtcaggatcatccgggcggcatcaggaggaagaggagcgtgagccacggaagcggaggaagcggagag
ggcaggggaagtcttctaacatgcggggacgtggaggaaaatcccggccccatgatcaagatcgccaccaggaagtacctg
ggcaagcagaacgtgtacgacatcggcgtggagagggaccacaacttcgccctgaagaacggcttcatcgccagcaactgta
gggagttgatttcagactatgcaccagattgttttgtgttaatagaaatgaatggcaagttagtcaaaactacgattgaagaactcat
tccactcaaatatacccgaaattaacccctctccctcccccccccctaacgttactggccgaagccgcttggaataaggccggtgt
gcgtttgtctatatgttattttccaccatattgccgtcttttggcaatgtgagggcccggaaacctggccctgtcttcttgacgagcattcc
taggggtctttcccctctcgccaaaggaatgcaaggtctgttgaatgtcgtgaaggaagcagttcctctggaagcttcttgaagaca
aacaacgtctgtagcgaccctttgcaggcagcggaaccccccacctggcgacaggtgcctctgcggccaaaagccacgtgtat
aagatacacctgcaaaggcggcacaaccccagtgccacgttgtgagttggatagttgtggaaagagtcaaatggctctcctcaa
gcgtattcaacaaggggctgaaggatgcccagaaggtaccccattgtatgggatctgatctggggcctcggtacacatgctttac
atgtgtttagtcgaggttaaaaaaacgtctaggccccccgaaccacggggacgtggttttcctttgaaaaacacgatgataatatg
gccacaaccatggtgagcaagggcgaggagctgttcaccggggtggtgcccatcctggtcgagctggacggcgacgtaaac
ggccacaagttcagcgtgtccggcgagggcgagggcgatgccacctacggcaagctgaccctgaagttcatctgcaccaccg
gcaagctgcccgtgccctggcccaccctcgtgaccaccctgacctacggcgtgcagtgcttcagccgctaccccgaccacatga
agcagcacgacttcttcaagtccgccatgcccgaaggctacgtccaggagcgcaccatcttcttcaaggacgacggcaactac
aagacccgcgccgaggtgaagttcgagggcgacaccctggtgaaccgcatcgagctgaagggcatcgacttcaaggagga
cggcaacatcctggggcacaagctggagtacaactacaacagccacaacgtctatatcatggccgacaagcagaagaacgg
catcaaggtgaacttcaagatccgccacaacatcgaggacggcagcgtgcagctcgccgaccactaccagcagaacacccc
catcggcgacggccccgtgctgctgcccgacaaccactacctgagcacccagtccgccctgagcaaagaccccaacgagaa
gcgcgatcacatggtcctgctggagttcgtgaccgccgccgggatcactctcggcatggacgagctgtacaagtaaacccagct
ttcttgtacaaagtggtgataatcgaattccgataatcaacctctggattacaaaatttgtgaaagattgactggtattcttaactatgtt
gctccttttacgctatgtggatacgctgctttaatgcctttgtatcatgctattgcttcccgtatggctttcattttctcctccttgtataaatcct
ggttgctgtctctttatgaggagttgtggcccgttgtcaggcaacgtggcgtggtgtgcactgtgtttgctgacgcaacccccactggt
tggggcattgccaccacctgtcagctcctttccgggactttcgctttccccctccctattgccacggcggaactcatcgccgcctgcct
tgcccgctgctggacaggggctcggctgttgggcactgacaattccgtggtgttgtcggggaagctgacgtcctttccatggctgct
cgcctgtgttgccacctggattctgcgcgggacgtccttctgctacgtcccttcggccctcaatccagcggaccttccttcccgcggc
ctgctgccggctctgcggcctcttccgcgtcttcgccttcgccctcagacgagtcggatctccctttgggccgcctccccgcatcggg
aattcccgcggttcgaattctaccgggtaggggaggcgcttttcccaaggcagtctggagcatgcgctttagcagccccgctggg
cacttggcgctacacaagtggcctctggcctcgcacacattccacatccaccggtaggcgccaaccggctccgttctttggtggcc
ccttcgcgccaccttctactcctcccctagtcaggaagttcccccccgccccgcagctcgcgtcgtgcaggacgtgacaaatgga
agtagcacgtctcactagtctcgtgcagatggacagcaccgctgagcaatggaagcgggtaggcctttggggcagcggccaat
agcagctttgctccttcgctttctgggctcagaggctgggaaggggtgggtccggggggggctcaggggcgggctcaggggc
ggggcgggcgcccgaaggtcctccggaggcccggcattctgcacgcttcaaaagcgcacgtctgccgcgctgttctcctcttcct
catctccgggcctttcgacctcacgtggccaccatgaaaaagcctgaactcaccgcgacgtctgtcgagaagtttctgatcgaaa
agttcgacagcgtctccgacctgatgcagctctcggagggcgaagaatctcgtgctttcagcttcgatgtaggagggcgtggatat
gtcctgcgggtaaatagctgcgccgatggtttctacaaagatcgttatgtttatcggcactttgcatcggccgcgctcccgattccgg
aagtgcttgacattggggaatttagcgagagcctgacctattgcatctcccgccgtgcacagggtgtcacgttgcaagacctgcct
gaaaccgaactgcccgctgttctgcagccggtcgcggaggccatggatgcgatcgctgcggccgatcttagccagacgagcg
ggttcggcccattcggaccgcaaggaatcggtcaatacactacatggcgtgatttcatatgcgcgattgctgatccccatgtgtatc
actggcaaactgtgatggacgacaccgtcagtgcgtccgtcgcgcaggctctcgatgagctgatgctttgggccgaggactgcc
ccgaagtccggcacctcgtgcacgcggatttcggctccaacaatgtcctgacggacaatggccgcataacagcggtcattgact
ggagcgaggcgatgttcggggattcccaatacgaggtcgccaacatcttcttctggaggccgtggttggcttgtatggagcagca
gacgcgctacttcgagcggaggcatccggagcttgcaggatcgccgcggctccgggcgtatatgctccgcattggtcttgacca
actctatcagagcttggttgacggcaatttcgatgatgcagcttgggcgcagggtcgatgcgacgcaatcgtccgatccggagcc
gggactgtcgggcgtacacaaatcgcccgcagaagcgcggccgtctggaccgatggctgtgtagaagtactcgccgatagtg
gaaaccgacgccccagcactcgtccgagggcaaaggaatagggtacctttaagaccaatgacttacaaggcagctgtagatc
ttagccactttttaaaagaaaaggggggactggaagggctaattcactcccaacgaagacaagatctgctttttgcttgtactgggt
ctctctggttagaccagatctgagcctgggagctctctggctaactagggaacccactgcttaagcctcaataaagcttgccttgag
tgcttcaagtagtgtgtgcccgtctgttgtgtgactctggtaactagagatccctcagacccttttagtcagtgtggaaaatctctagca
EF-1a promoter
(SEQ ID NO: 16)
Ggctccggtgcccgtcagtgggcagagcgcacatcgcccacagtccccgagaagttggggggaggggtcggcaattgaacc
ggtgcctagagaaggtggcgcggggtaaactgggaaagtgatgtcgtgtactggctccgcctttttcccgaggggggggagaa
ccgtatataagtgcagtagtcgccgtgaacgttctttttcgcaacgggtttgccgccagaacacaggtaagtgccgtgtgtggttccc
gcgggcctggcctctttacgggttatggcccttgcgtgccttgaattacttccacctggctgcagtacgtgattcttgatcccgagcttc
gggttggaagtggggggagagttcgaggccttgcgcttaaggagccccttcgcctcgtgcttgagttgaggcctggcctgggcg
ctggggccgccgcgtgcgaatctggtggcaccttcgcgcctgtctcgctgctttcgataagtctctagccatttaaaatttttgatgac
ctgctgcgacgctttttttctggcaagatagtcttgtaaatgcgggccaagatctgcacactggtatttcggtttttggggccgcgggc
ggcgacggggcccgtgcgtcccagcgcacatgttcggcgaggcggggcctgcgagcgcggccaccgagaatcggacggg
ggtagtctcaagctggccggcctgctctggtgcctggtctcgcgccgccgtgtatcgccccgccctgggcggcaaggctggcccg
gtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagta
ccgggcgccgtccaggcacctcgattagttctcgagcttttggagtacgtcgtctttaggttggggggaggggttttatgcgatggag
tttccccacactgagtgggtggagactgaagttaggccagcttggcacttgatgtaattctccttggaatttgccctttttgagtttggat
cttggttcattctcaagcctcagacagtggttcaaagtttttttcttccatttcaggtgtcgtga
PGK promoter
(SEQ ID NO: 17)
Gggtaggggaggcgcttttcccaaggcagtctggagcatgcgctttagcagccccgctgggcacttggcgctacacaagtggc
ctctggcctcgcacacattccacatccaccggtaggcgccaaccggctccgttctttggtggccccttcgcgccaccttctactcctc
ccctagtcaggaagttcccccccgccccgcagctcgcgtcgtgcaggacgtgacaaatggaagtagcacgtctcactagtctcg
tgcagatggacagcaccgctgagcaatggaagcgggtaggcctttggggcagcggccaatagcagctttgctccttcgctttctg
ggctcagaggctgggaaggggtgggtccggggggggctcaggggcgggctcaggggggggcgggcgcccgaaggtcc
tccggaggcccggcattctgcacgcttcaaaagcgcacgtctgccgcgctgttctcctcttcctcatctccgggcctttcg
CMV enhancer and promoter
(SEQ ID NO: 18)
Cgttacataacttacggtaaatggcccgcctggctgaccgcccaacgacccccgcccattgacgtcaataatgacgtatgttccc
atagtaacgccaatagggactttccattgacgtcaatgggtggagtatttacggtaaactgcccacttggcagtacatcaagtgtat
catatgccaagtacgccccctattgacgtcaatgacggtaaatggcccgcctggcattatgcccagtacatgaccttatgggacttt
cctacttggcagtacatctacgtattagtcatcgctattaccatggtgatgcggttttggcagtacatcaatgggcgtggatagcggttt
gactcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagagct
3xstop codon
(SEQ ID NO: 19)
Tgataatag
2 x 3 stop codon
(SEQ ID NO: 20)
Tgataatagctgataatag
split BsrR including inteins (protein sequence from retargeted nucleic acid)
(SEQ ID NO: 21)
MKTFNISQQDLELVEVATEKITMLYEDNKHHVGAAIRTKTGEIISAVHIEAYIGRVTVCAEAIAIGSAVS
NGQKDFDTIVAVRHPYSDEVDRSIRVVSPCGMCLSYETEILTVEYGLLPIGKIVEKRIECTVYSVDNNG
NIYTQPVAQWHDRGEQEVFEYCLEDGSLIRATKDHKFMTVDGQMLPIDEIFERELDLMRVDNLPNGIR
RKRSVSHGSGEGRGSLLTCGDVEENPGPVVVCLVNHRGLRRQDHPGGIRRKRSVSHGSGGSGEG
RGSLLTCGDVEENPGPMIKIATRKYLGKQNVYDIGVERDHNFALKNGFIASNCRELISDYAPDCFVLIE
MNGKLVKTTIEELIPLKYTRN
The N-terminal part of the split selectable marker and the C-terminal part of the split
selectable marker are highlighted in bold.
Furin-T2A linker protein sequence (pPlatform)
(SEQ ID NO: 22)
GIRRKRSVSHGSGGSG
Furin-T2A linker protein sequence (pDeliver)
(SEQ ID NO: 23)
GIRRKRSVSHGSG
SV40 promoter
(SEQ ID NO: 24)
CTGTGGAATGTGTGTCAGTTAGGGTGTGGAAAGTCCCCAGGCTCCCCAGCAGGCAGAA
GTATGCAAAGCATGCATCTCAATTAGTCAGCAACCAGGTGTGGAAAGTCCCCAGGCTC
CCCAGCAGGCAGAAGTATGCAAAGCATGCATCTCAATTAGTCAGCAACCATAGTCCCG
CCCCTAACTCCGCCCATCCCGCCCCTAACTCCGCCCAGTTCCGCCCATTCTCCGCCCC
ATGGCTGACTAATTTTTTTTATTTATGCAGAGGCCGAGGCCGCCTCTGCCTCTGAGCTA
TTCCAGAAGTAGTGAGGAGGCTTTTTTGGAGGCCTAGGCTTTTGCAAAAAGCT
FOLR1A coding sequence
(SEQ ID NO: 25)
ATGGCCCAGAGGATGACCACCCAGCTGCTGCTGCTGCTGGTGTGGGTGGCCGTGGTG
GGCGAGGCCCAGACCAGGATCGCCTGGGCCAGGACCGAGCTGCTGAACGTGTGCATG
AACGCCAAGCACCACAAGGAGAAGCCCGGCCCCGAGGACAAGCTGCACGAGCAGTGC
AGGCCCTGGAGGAAGAACGCCTGCTGCAGCACCAACACCAGCCAGGAGGCCCACAAG
GACGTGAGCTACCTGTACAGGTTCAACTGGAACCACTGCGGCGAGATGGCCCCCGCC
TGCAAGAGGCACTTCATCCAGGACACCTGCCTGTACGAGTGCAGCCCCAACCTGGGCC
CCTGGATCCAGCAGGTGGACCAGAGCTGGAGGAAGGAGAGGGTGCTGAACGTGCCCC
TGTGCAAGGAGGACTGCGAGCAGTGGTGGGAGGACTGCAGGACCAGCTACACCTGCA
AGAGCAACTGGCACAAGGGCTGGAACTGGACCAGCGGCTTCAACAAGTGCGCCGTGG
GCGCCGCCTGCCAGCCCTTCCACTTCTACTTCCCCACCCCCACCGTGCTGTGCAACGA
GATCTGGACCCACAGCTACAAGGTGAGCAACTACAGCAGGGGCAGCGGCAGGTGCAT
CCAGATGTGGTTCGACCCCGCCCAGGGCAACCCCAACGAGGAGGTGGCCAGGTTCTA
CGCCGCCGCCATGAGCGGCGCCGGCCCCTGGGCCGCCTGGCCCTTCCTGCTGAGCC
TGGCCCTGATGCTGCTGTGGCTGCTGAGCTGA
CD19 coding sequence
(SEQ ID NO: 26)
ATGCCACCTCCTCGCCTCCTCTTCTTCCTCCTCTTCCTCACCCCCATGGAAGTCAGGCC
CGAGGAACCTCTAGTGGTGAAGGTGGAAGAGGGAGATAACGCTGTGCTGCAGTGCCT
CAAGGGGACCTCAGATGGCCCCACTCAGCAGCTGACCTGGTCTCGGGAGTCCCCGCT
TAAACCCTTCTTAAAACTCAGCCTGGGGCTGCCAGGCCTGGGAATCCACATGAGGCCC
CTGGCCATCTGGCTTTTCATCTTCAACGTCTCTCAACAGATGGGGGGCTTCTACCTGTG
CCAGCCGGGGCCCCCCTCTGAGAAGGCCTGGCAGCCTGGCTGGACAGTCAATGTGGA
GGGCAGCGGGGAGCTGTTCCGGTGGAATGTTTCGGACCTAGGTGGCCTGGGCTGTGG
CCTGAAGAACAGGTCCTCAGAGGGCCCCAGCTCCCCTTCCGGGAAGCTCATGAGCCC
CAAGCTGTATGTGTGGGCCAAAGACCGCCCTGAGATCTGGGAGGGAGAGCCTCCGTG
TCTCCCACCGAGGGACAGCCTGAACCAGAGCCTCAGCCAGGACCTCACCATGGCCCC
TGGCTCCACACTCTGGCTGTCCTGTGGGGTACCCCCTGACTCTGTGTCCAGGGGCCCC
CTCTCCTGGACCCATGTGCACCCCAAGGGGCCTAAGTCATTGCTGAGCCTAGAGCTGA
AGGACGATCGCCCGGCCAGAGATATGTGGGTAATGGAGACGGGTCTGTTGTTGCCCC
GGGCCACAGCTCAAGACGCTGGAAAGTATTATTGTCACCGTGGCAACCTGACCATGTC
ATTCCACCTGGAGATCACTGCTCGGCCAGTACTATGGCACTGGCTGCTGAGGACTGGT
GGCTGGAAGGTCTCAGCTGTGACTTTGGCTTATCTGATCTTCTGCCTGTGTTCCCTTGT
GGGCATTCTTCATCTTCAAAGAGCCCTGGTCCTGAGGAGGAAAAGAAAGCGAATGACT
GACCCCACCAGGAGATGA
pDeliver FOLR1A construct
(SEQ ID NO: 27)
caactttgtatagaaaagttgggcctaactggccggtaccgagtttctagacggagtactgtcctccgagcggagtactgtcctccg
actcgagcggagtactgtcctccgagcggagtactgtcctccgagcggagtactgtcctccgagcggagtactgtcctccgagcg
gagtactgtcctccgagcggagtactgtcctccgaggaattccggagtactgtcctccgaagacgctagcggggggctataaaa
gggggtgggggcgttcgtcctcactctagatctgcgatctaagtaagcttggcaatccggtactgttggtaaacaagtttgtacaaa
aaagcaggctgccaccatggcccagaggatgaccacccagctgctgctgctgctggtgtgggtggccgtggtgggcgaggcc
cagaccaggatcgcctgggccaggaccgagctgctgaacgtgtgcatgaacgccaagcaccacaaggagaagcccggcc
ccgaggacaagctgcacgagcagtgcaggccctggaggaagaacgcctgctgcagcaccaacaccagccaggaggccc
acaaggacgtgagctacctgtacaggttcaactggaaccactgcggcgagatggcccccgcctgcaagaggcacttcatcca
ggacacctgcctgtacgagtgcagccccaacctgggcccctggatccagcaggtggaccagagctggaggaaggagagggt
gctgaacgtgcccctgtgcaaggaggactgcgagcagtggtgggaggactgcaggaccagctacacctgcaagagcaactg
gcacaagggctggaactggaccagcggcttcaacaagtgcgccgtgggcgccgcctgccagcccttccacttctacttccccac
ccccaccgtgctgtgcaacgagatctggacccacagctacaaggtgagcaactacagcaggggcagcggcaggtgcatcca
gatgtggttcgaccccgcccagggcaaccccaacgaggaggtggccaggttctacgccgccgccatgagcggcgccggccc
ctgggccgcctggcccttcctgctgagcctggccctgatgctgctgtggctgctgagctgaggccgcgactctagagtcggggcg
gccggccgcttcgagcagacatgataagatacattgatgagtttggacaaaccacaactagaatgcagtgaaaaaaatgcttta
tttgtgaaatttgtgatgctattgctttatttgtaaccattataagctgcaataaacaagttaacaacaacaattgcattcattttatgtttca
ggttcagggggaggtgtgggaggttttttaaagcaagtaaaacctctacaaatgtggtaaaatcgataagcatccgtttgcgtattg
ggcgctcttccgctgatctgcgcagcaccatgttctacccgttacataacttacggtaaatggcccgcctggctgaccgcccaacg
acccccgcccattgacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacggtaaatggccc
gcctggcattatgcccagtacatgaccttatgggactttcctacttggcagtacatctacgtattagtcatcgctattaccatggtgatg
cggttttggcagtacatcaatgggcgtggatagcggtttgactcacggggatttccaagtctccaccccattgacgtcaatgggagt
ttgttttggcaccaaaatcaacgggactttccaaaatgtcgtaacaactccgccccattgacgcaaatgggcggtaggcgtgtac
ggtgggaggtctatataagcagagctctctggctaactgtcgggatcaccgaattcaccggtgccgccaccatgaaaacatttaa
catttctcaacaggatctagaattagtagaagtagcgacagagaagattacaatgctttatgaggataataaacatcatgtggga
gcggcaattcgtacgaaaacaggagaaatcatttcggcagtacatattgaagcgtatataggacgagtaactgtttgtgcagaag
ccattgcgattggtagtgcagtttcgaatggacaaaaggattttgacacgattgtagctgttagacacccttattctgacgaagtaga
tagaagtattcgagtggtaagtccttgtggtatgtgcctgagctacgagaccgagatcctgaccgtggagtacggcctgctgccca
tcggcaagatcgtggagaagaggatcgagtgcaccgtgtacagcgtggacaacaacggcaacatctacacccagcccgtgg
cccagtggcacgacaggggcgagcaggaggtgttcgagtactgcctggaggacggcagcctgatcagggccaccaaggac
cacaagttcatgaccgtggacggccagatgctgcccatcgacgagatcttcgagagggagctggacctgatgagggtggaca
acctgcccaacggcatcaggaggaagaggagcgtgagccacggaagcggagagggcaggggaagtcttctaacatgcgg
ggacgtggaggaaaatcccggccccgtcgtggtttgtctggtcaaccaccgcggtctcagtggtgtacggtacaaaccccgacg
gtaaacccagctttcttgtacaaagtggtgatggccggccgcttcgagcagacatgataagatacattgatgagtttggacaaacc
acaactagaatgcagtgaaaaaaatgctttatttgtgaaatttgtgatgctattgctttatttgtaaccattataagctgcaataaacaa
gttaacaacaacaattgcattcattttatgtttcaggttcagggggaggtgtgggaggttttttaaagcaagtaaaacctctacaaat
gtggtagcggccgcggcgctcttccgcttcctcgctcactgactcgctgcgctcggtcgttcggctgcggcgagcggtatcagctc
actcaaaggcggtaatacggttatccacagaatcaggggataacgcaggaaagaacatgtgagcaaaaggccagcaaaag
gccaggaaccgtaaaaaggccgcgttgctggcgtttttccataggctccgcccccctgacgagcatcacaaaaatcgacgctca
agtcagaggtggcgaaacccgacaggactataaagataccaggcgtttccccctggaagctccctcgtgcgctctcctgttccga
ccctgccgcttaccggatacctgtccgcctttctctcttcgggaagcgtggcgctttctcatagctcacgctgtaggtatctcagttcgg
tgtaggtcgttcgctccaagctgggctgtgtgcacgaaccccccgttcagcccgaccgctgcgccttatccggtaactatcgtcttg
agtccaacccggtaagacacgacttatcgccactggcagcagccactggtaacaggattagcagagcgaggtatgtaggcgg
tgctacagagttcttgaagtggtggcctaactacggctacactagaagaacagtatttggtatctgcgctctgctgaagccagttac
cttcggaaaaagagttggtagctcttgatccggcaaacaaaccaccgctggtagcggtggtttttttgtttgcaagcagcagattac
gcgcagaaaaaaaggatctcaagaagatcctttgatcttttctacggggtctgacgctcagtggaacgaaaactcacgttaagg
gattttggtcatgagattatcaaaaaggatcttcacctagatccttttaaattaaaaatgaagttttaaatcaatctaaagtatatatga
gtaaacttggtctgacagttaccaatgcttaatcagtgaggcacctatctcagcgatctgtctatttcgttcatccatagttgcctgactc
cccgtcgtgtagataactacgatacgggagggcttaccatctggccccagtgctgcaatgataccgcgagacccacgctcaccg
gctccagatttatcagcaataaaccagccagccggaagggccgagcgcagaagtggtcctgcaactttatccgcctccatccag
tctattaattgttgccgggaagctagagtaagtagttcgccagttaatagtttgcgcaacgttgttgccattgctacaggcatcgtggt
gtcacgctcgtcgtttggtatggcttcattcagctccggttcccaacgatcaaggcgagttacatgatcccccatgttgtgcaaaaaa
gcggttagctccttcggtcctccgatcgttgtcagaagtaagttggccgcagtgttatcactcatggttatggcagcactgcataattct
cttactgtcatgccatccgtaagatgcttttctgtgactggtgagtactcaaccaagtcattctgagaatagtgtatgcggcgaccga
gttgctcttgcccggcgtcaatacgggataataccgcgccacatagcagaactttaaaagtgctcatcattggaaaacgttcttcgg
ggcgaaaactctcaaggatcttaccgctgttgagatccagttcgatgtaacccactcgtgcacccaactgatcttcagcatcttttac
tttcaccagcgtttctgggtgagcaaaaacaggaaggcaaaatgccgcaaaaaagggaataagggcgacacggaaatgttg
aatactcatactcttcctttttcaatattattgaagcatttatcagggttattgtctcatgagcggatacatatttgaatgtatttagaaaaat
aaacaaataggggttccgcgcacatttccccgaaaagtgccacctgacgtctaagaaaccattattatcatgacattaacctata
aaaataggcgtatcacgaggccctttcgtcggcgcgccgcggccgc
FOLR1A coding sequence is underlined.
pDeliver CD19 construct
(SEQ ID NO: 28)
caactttgtatagaaaagttgggcctaactggccggtaccgagtttctagacggagtactgtcctccgagcggagtactgtcctccg
actcgagcggagtactgtcctccgagcggagtactgtcctccgagcggagtactgtcctccgagcggagtactgtcctccgagcg
gagtactgtcctccgagcggagtactgtcctccgaggaattccggagtactgtcctccgaagacgctagcggggggctataaaa
gggggtgggggcgttcgtcctcactctagatctgcgatctaagtaagcttggcaatccggtactgttggtaaacaagtttgtacaaa
aaagcaggctgccaccatgccacctcctcgcctcctcttcttcctcctcttcctcacccccatggaagtcaggcccgaggaacctct
agtggtgaaggtggaagagggagataacgctgtgctgcagtgcctcaaggggacctcagatggccccactcagcagctgacc
tggtctcgggagtccccgcttaaacccttcttaaaactcagcctggggctgccaggcctgggaatccacatgaggcccctggcca
tctggcttttcatcttcaacgtctctcaacagatggggggcttctacctgtgccagccggggcccccctctgagaaggcctggcagc
ctggctggacagtcaatgtggagggcagcggggagctgttccggtggaatgtttcggacctaggtggcctgggctgtggcctga
agaacaggtcctcagagggccccagctccccttccgggaagctcatgagccccaagctgtatgtgtgggccaaagaccgccct
gagatctgggagggagagcctccgtgtctcccaccgagggacagcctgaaccagagcctcagccaggacctcaccatggcc
cctggctccacactctggctgtcctgtggggtaccccctgactctgtgtccaggggccccctctcctggacccatgtgcaccccaa
ggggcctaagtcattgctgagcctagagctgaaggacgatcgcccggccagagatatgtgggtaatggagacgggtctgttgtt
gccccgggccacagctcaagacgctggaaagtattattgtcaccgtggcaacctgaccatgtcattccacctggagatcactgct
cggccagtactatggcactggctgctgaggactggtggctggaaggtctcagctgtgactttggcttatctgatcttctgcctgtgttcc
cttgtgggcattcttcatcttcaaagagccctggtcctgaggaggaaaagaaagcgaatgactgaccccaccaggagatgagg
ccgcgactctagagtcggggcggccggccgcttcgagcagacatgataagatacattgatgagtttggacaaaccacaactag
aatgcagtgaaaaaaatgctttatttgtgaaatttgtgatgctattgctttatttgtaaccattataagctgcaataaacaagttaacaa
caacaattgcattcattttatgtttcaggttcagggggaggtgtgggaggttttttaaagcaagtaaaacctctacaaatgtggtaaa
atcgataagcatccgtttgcgtattgggcgctcttccgctgatctgcgcagcaccatgttctacccgttacataacttacggtaaatgg
cccgcctggctgaccgcccaacgacccccgcccattgacgtcaataatgacgtatgttcccatagtaacgccaatagggactttc
cattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctacgtat
tagtcatcgctattaccatggtgatgcggttttggcagtacatcaatgggcgtggatagcggtttgactcacggggatttccaagtctc
caccccattgacgtcaatgggagtttgttttggcaccaaaatcaacgggactttccaaaatgtcgtaacaactccgccccattgac
gcaaatgggcggtaggcgtgtacggtgggaggtctatataagcagagctctctggctaactgtcgggatcaccgaattcaccggt
gccgccaccatgaaaacatttaacatttctcaacaggatctagaattagtagaagtagcgacagagaagattacaatgctttatg
aggataataaacatcatgtgggagcggcaattcgtacgaaaacaggagaaatcatttcggcagtacatattgaagcgtatatag
gacgagtaactgtttgtgcagaagccattgcgattggtagtgcagtttcgaatggacaaaaggattttgacacgattgtagctgtta
gacacccttattctgacgaagtagatagaagtattcgagtggtaagtccttgtggtatgtgcctgagctacgagaccgagatcctg
accgtggagtacggcctgctgcccatcggcaagatcgtggagaagaggatcgagtgcaccgtgtacagcgtggacaacaac
ggcaacatctacacccagcccgtggcccagtggcacgacaggggcgagcaggaggtgttcgagtactgcctggaggacggc
agcctgatcagggccaccaaggaccacaagttcatgaccgtggacggccagatgctgcccatcgacgagatcttcgagaggg
agctggacctgatgagggtggacaacctgcccaacggcatcaggaggaagaggagcgtgagccacggaagcggagagg
gcaggggaagtcttctaacatgcggggacgtggaggaaaatcccggccccgtcgtggtttgtctggtcaaccaccgcggtctca
gtggtgtacggtacaaaccccgacggtaaacccagctttcttgtacaaagtggtgatggccggccgcttcgagcagacatgata
agatacattgatgagtttggacaaaccacaactagaatgcagtgaaaaaaatgctttatttgtgaaatttgtgatgctattgctttattt
gtaaccattataagctgcaataaacaagttaacaacaacaattgcattcattttatgtttcaggttcagggggaggtgtgggaggttt
tttaaagcaagtaaaacctctacaaatgtggtagcggccgcggcgctcttccgcttcctcgctcactgactcgctgcgctcggtcgtt
cggctgcggcgagcggtatcagctcactcaaaggcggtaatacggttatccacagaatcaggggataacgcaggaaagaac
atgtgagcaaaaggccagcaaaaggccaggaaccgtaaaaaggccgcgttgctggcgtttttccataggctccgcccccctga
cgagcatcacaaaaatcgacgctcaagtcagaggtggcgaaacccgacaggactataaagataccaggcgtttccccctgga
agctccctcgtgcgctctcctgttccgaccctgccgcttaccggatacctgtccgcctttctctcttcgggaagcgtggcgctttctcat
agctcacgctgtaggtatctcagttcggtgtaggtcgttcgctccaagctgggctgtgtgcacgaaccccccgttcagcccgaccg
ctgcgccttatccggtaactatcgtcttgagtccaacccggtaagacacgacttatcgccactggcagcagccactggtaacagg
attagcagagcgaggtatgtaggcggtgctacagagttcttgaagtggtggcctaactacggctacactagaagaacagtatttg
gtatctgcgctctgctgaagccagttaccttcggaaaaagagttggtagctcttgatccggcaaacaaaccaccgctggtagcgg
tggtttttttgtttgcaagcagcagattacgcgcagaaaaaaaggatctcaagaagatcctttgatcttttctacggggtctgacgctc
agtggaacgaaaactcacgttaagggattttggtcatgagattatcaaaaaggatcttcacctagatccttttaaattaaaaatgaa
gttttaaatcaatctaaagtatatatgagtaaacttggtctgacagttaccaatgcttaatcagtgaggcacctatctcagcgatctgt
ctatttcgttcatccatagttgcctgactccccgtcgtgtagataactacgatacgggagggcttaccatctggccccagtgctgcaa
tgataccgcgagacccacgctcaccggctccagatttatcagcaataaaccagccagccggaagggccgagcgcagaagtg
gtcctgcaactttatccgcctccatccagtctattaattgttgccgggaagctagagtaagtagttcgccagttaatagtttgcgcaac
gttgttgccattgctacaggcatcgtggtgtcacgctcgtcgtttggtatggcttcattcagctccggttcccaacgatcaaggcgagtt
acatgatcccccatgttgtgcaaaaaagcggttagctccttcggtcctccgatcgttgtcagaagtaagttggccgcagtgttatcac
tcatggttatggcagcactgcataattctcttactgtcatgccatccgtaagatgcttttctgtgactggtgagtactcaaccaagtcatt
ctgagaatagtgtatgcggcgaccgagttgctcttgcccggcgtcaatacgggataataccgcgccacatagcagaactttaaa
agtgctcatcattggaaaacgttcttcggggcgaaaactctcaaggatcttaccgctgttgagatccagttcgatgtaacccactcgt
gcacccaactgatcttcagcatcttttactttcaccagcgtttctgggtgagcaaaaacaggaaggcaaaatgccgcaaaaaag
ggaataagggcgacacggaaatgttgaatactcatactcttcctttttcaatattattgaagcatttatcagggttattgtctcatgagc
ggatacatatttgaatgtatttagaaaaataaacaaataggggttccgcgcacatttccccgaaaagtgccacctgacgtctaag
aaaccattattatcatgacattaacctataaaaataggcgtatcacgaggccctttcgtcggcgcgccgcggccgc
CD19 coding sequence is underlined.

### Example 2—Experimental Data

### Construct Design

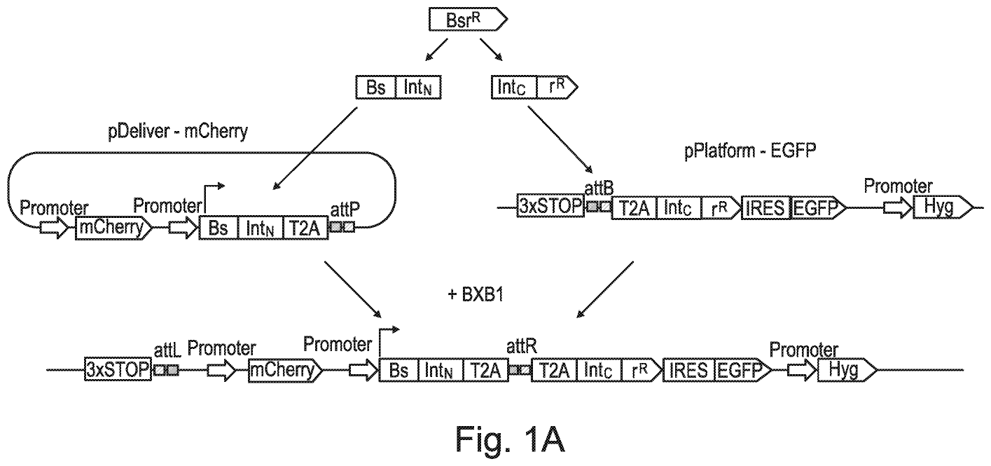

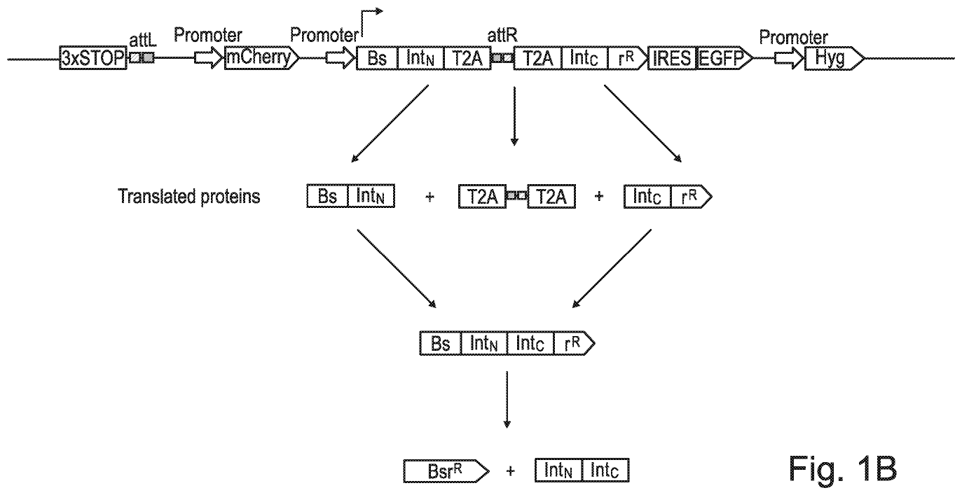

The targeted integration and selection system according to one preferred embodiment of the present invention is shown in FIG. 1A and FIG. 1B.

In order to select for retargeting events at the landing pad, the system was designed so that the blasticidin S deaminase (Bsr^{R}) sequence is split in two, with half in the landing pad (pPlatform vector) and half in the retargeting (pDeliver) plasmid. The blasticidin S deaminase (Bsr^{R}) is split between amino acids 102 and 103. Only a successful recombination event (integration at the landing pad in a specific orientation and position) would result in the translation of a full Bsr^{R} protein.

The platform system makes use of the NpuDnaE split intein system from Nostoc punctiforme which is capable of performing highly efficient protein trans-splicing. Trans-splicing is a special form of protein processing where two different primary proteins are joined end to end. Using the NpuDnaE split intein system means that there is no need to make insertions in the coding sequence of Bsr^{R}.

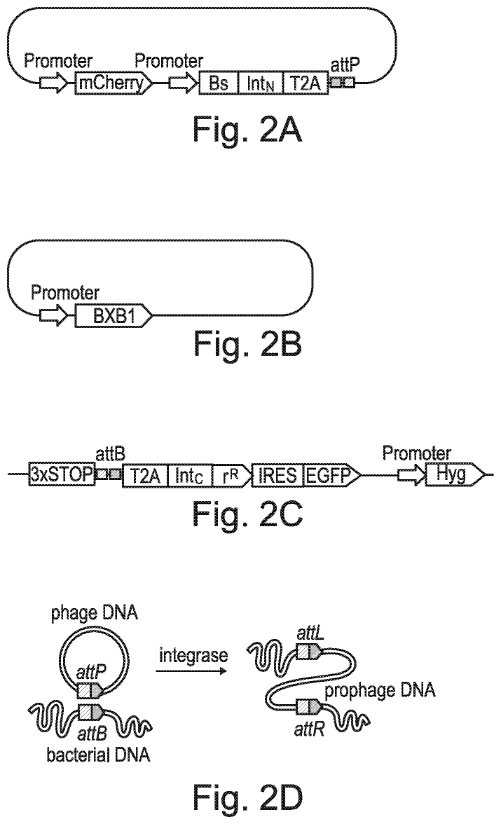

The platform system further makes use of BXB1 which is a phage derived serine recombinase which catalyses site specific recombination between attP (phage attachment) and attB (bacterial attachment) sites. The BXB1 integrase is capable of mediating highly efficient and accurate site-specific recombination in mammalian cells. By inserting a BXB1 recombination motif (attB in this example) into the genome of a cell line e.g. by lentiviral delivery, it is possible to create a platform cell line with a ‘landing pad’ which can be subsequently retargeted by transfecting the cell line with a plasmid bearing the corresponding recombination motif (attP) and a plasmid expressing BXB1. A schematic representation of the integration of phage DNA into host bacterial DNA by means of recombination between attachment sites catalysed by the phage integrase enzyme is shown in FIG. 2D.

The N-terminal portion (Bs) is attached to the N-terminal portion of the NpuDnaE split intein (Int_{N}) and incorporated into the pDeliver retargeting plasmid under a constitutive promoter. The Int_{N} coding sequence is followed in-frame by an optimized furin cleavage site/T2A peptide sequence, then by the BXB1 attP recombination motif. The C-terminal portion of Bsr^{R} (r^{R}) is attached downstream of the NpuDnaE split intein C-terminal portion (Int_{C}) and incorporated into the pPlatform construct, downstream of the BXB1 attB motif and another optimized furin cleavage site/T2A peptide sequence. Optimized T2A self-cleaving peptide sequences combined with optimized furin cleavage site motifs ensure that the two halves of the system are translated as free proteins.

To avoid transcription and translation of the C-terminal half of the Bsr^{R} present in the platform integration site in the absence of retargeting, three stop codons were placed upstream of the coding sequence. Retargeting at the BXB1 attB motif causes these stop codons to be moved upstream of the promoter for the cargo of the pDeliver plasmid (mCherry).

Transcription and translation of the C-terminal portion are prevented by in-frame stop codons upstream of the coding sequences. Upon BXB1 mediated recombination, the two ‘halves’ of the Bsr^{R} are recombined as one single coding sequence, including the intein sequences and translation of the recombined BXB1 attR site.

### Materials and Methods

### Cell Culture

All cell culture reagents and antibiotics were obtained from Thermo Fisher Scientific, Carlsbad, CA (e.g. Gibco and Invitrogen brands) unless otherwise specified. HEK293 cells were cultured in Dulbecco's modified Eagle's medium (DMEM) (Gibco) with 10% FBS (Gibco) and 1× penicillin/streptomycin (Pen/Strep; Gibco). CHO-K1 cells were cultured in RPMI 1640 with 10% FBS, 1× glutamate (Gibco), and 1× Pen/Strep.

Integration of pPlatform ‘Landing Pad’ Cassette

Lentiviral delivery of the pPlatform ‘landing pad’ construct was used. The construct was additionally designed with a hygromycin resistance gene to allow selection of stably transduced cells. HEK293 and CHO-K1 cells were seeded in 6 well plates and incubated for a period of 24 hrs before lentivirus containing the pPlatform ‘landing pad’ cassette was added at an MOI (ratio of the number of transducing lentiviral particles to the number of cell) of 15 in the presence of 8 μg/ml polybrene (Sigma-Aldrich: St. Louis, MO). Following a further incubation period of 48 hours, media containing hygromycin was added and the cells cultured for a period of 1 week in the selection medium. Following selection, the cells were stored at −180° C. for use as a base for further experiments. Cells in which the pPlatform ‘landing pad’ construct was stably integrated are expected to survive in the selection media due to the presence of hygromycin resistance gene in the construct.

### Co-Transfection of Retargeting Construct and BXB1 Expression Vector

HEK293 and CHO-K1 cells which have been selected for stable integration of pPlatform ‘landing pad’ construct as described above were seeded in 6 well plates and incubated for a period of 24 hrs before being transfected with a 1:4 ratio of pDeliver retargeting plasmid to the pIntegrase BXB1 expression plasmid, 2.5 μg total DNA, using Lipofectamine LTX transfection reagent (Invitrogen). Following a 48-hour incubation period, media containing blasticidin was added to the cells and the cells cultured for a period of 1 week in selection medium. Following selection, the cells were analysed for the expression of EGFP and mCherry via flow cytometry. Cells in which the pDeliver retargeting plasmid has been stably integrated in the correct position and orientation in the pPlatform ‘landing pad’ construct are expected to survive in the selection media due to the reconstruction of blasticidin resistance protein from:
the N-terminal portion of the blasticidin gene (Bs in FIGS. 1A and 1B) attached to the N-terminal portion of the NpuDnaE split intein (Int_{N} in FIGS. 1A and 1B) from the pDeliver retargeting construct and
the C-terminal portion of the blasticidin gene (r^{R} in FIGS. 1A and 1B) attached downstream of the NpuDnaE split intein C-terminal portion (Int_{C} in FIGS. 1A and 1B) from the pPlatform ‘landing pad’ construct.

BXB1 is driven by EF-1a promoter.

### Experimental Data

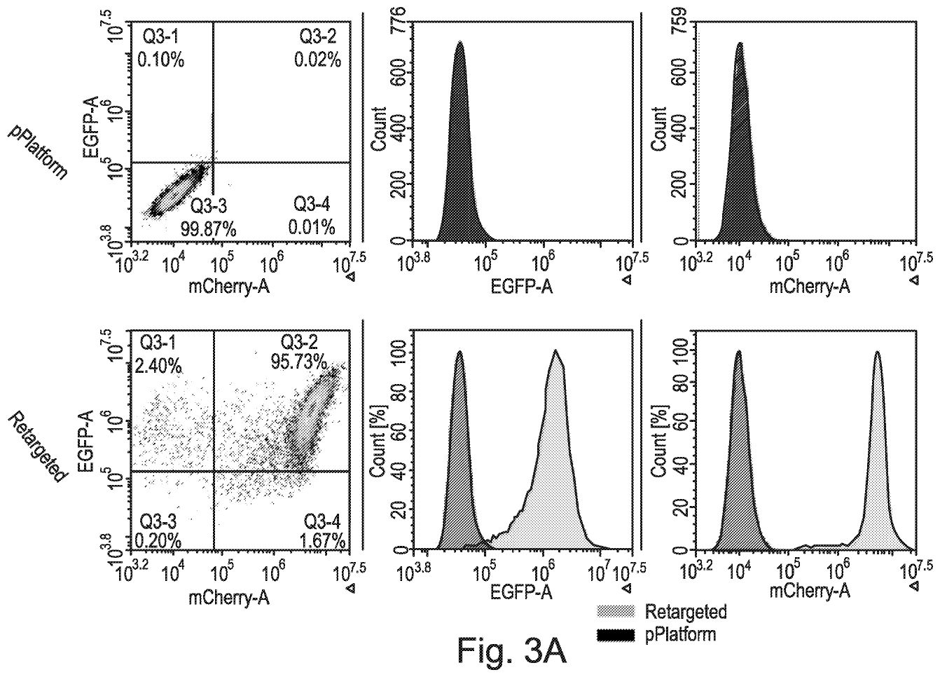

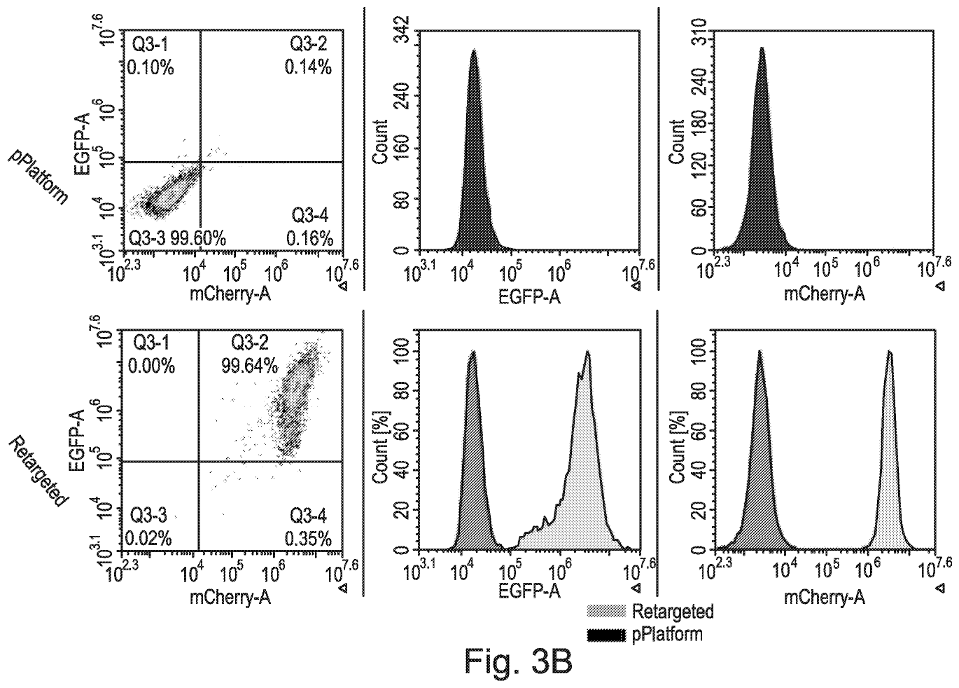

Following successful growth through both selection media described above, the retargeted cells were analysed by flow cytometry for mCherry and EGFP expression. The retargeted cells were compared to the parental platform pools which have undergone only the first media selection described above. The results of this experiment are shown in FIG. 3A and FIG. 3B for CHO-K1 cells and HEK293 cells, respectively.

The pPlatform ‘landing pad’ construct comprises EGFP which is downstream of an internal ribosome entry site downstream of the C-terminal portion of the blasticidin gene (r^{R} in FIGS. 1A and 1B) and the NpuDnaE split intein C-terminal portion (Int_{C} in FIGS. 1A and 1B). EGFP is not expected to be expressed unless the construct in FIG. 1B has been created following successful integration.

The pDeliver retargeting plasmid comprises mCherry operably linked to a promoter so is expected to be expressed provided that the pDeliver retargeting plasmid is present or integrated in the cell.

Cells which have been successfully retargeted are expected to express both mCherry and EGFP. As can clearly be seen in FIGS. 3A and 3B, the platform system can be successfully retargeted in both HEK293 and CHO-K1 cells. The EGFP expression confirms that successful integration of the pDeliver retargeting plasmid has taken place at the platform site within the pPlatform ‘landing pad’ construct as EGFP is only expressed in the case of successful retargeting. The expression profile of mCherry is that of a narrow peak suggesting that all the cells which have grown through the blasticidin selection have the same level of expression.

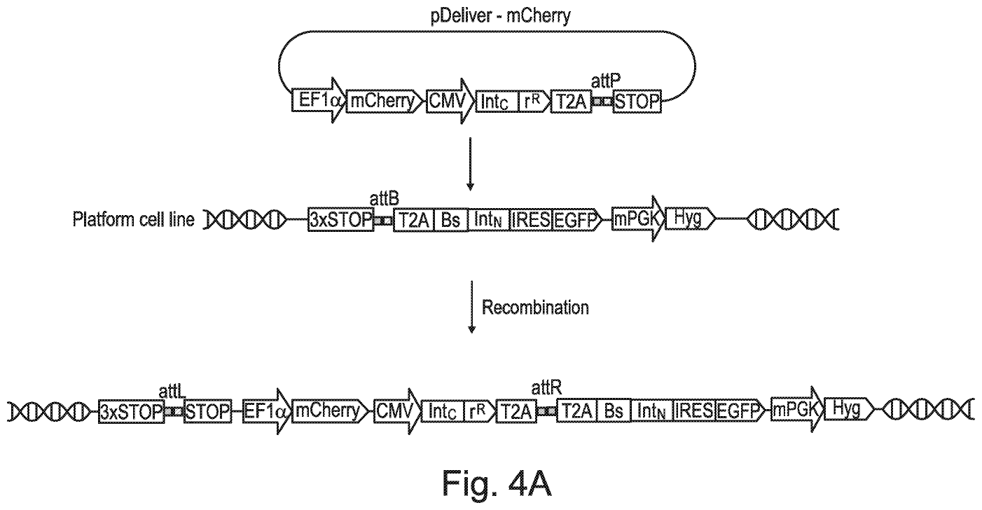

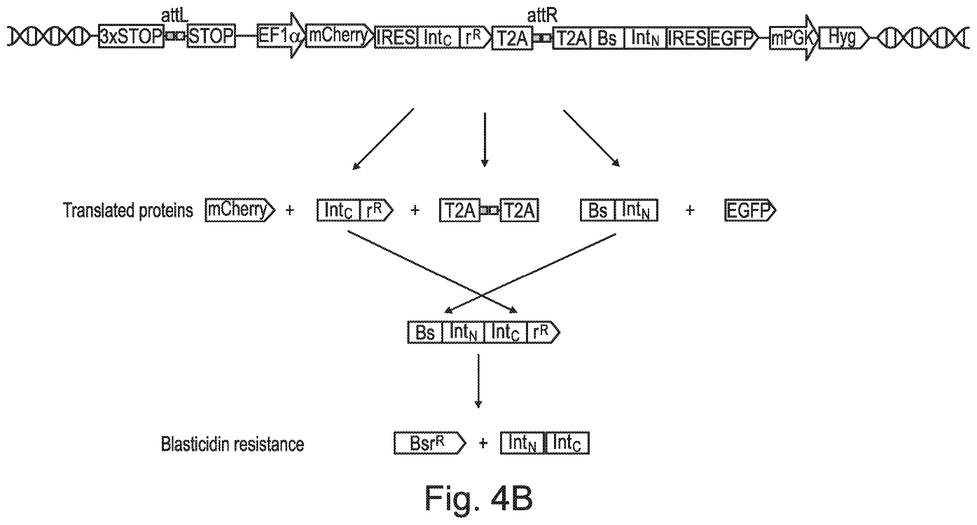

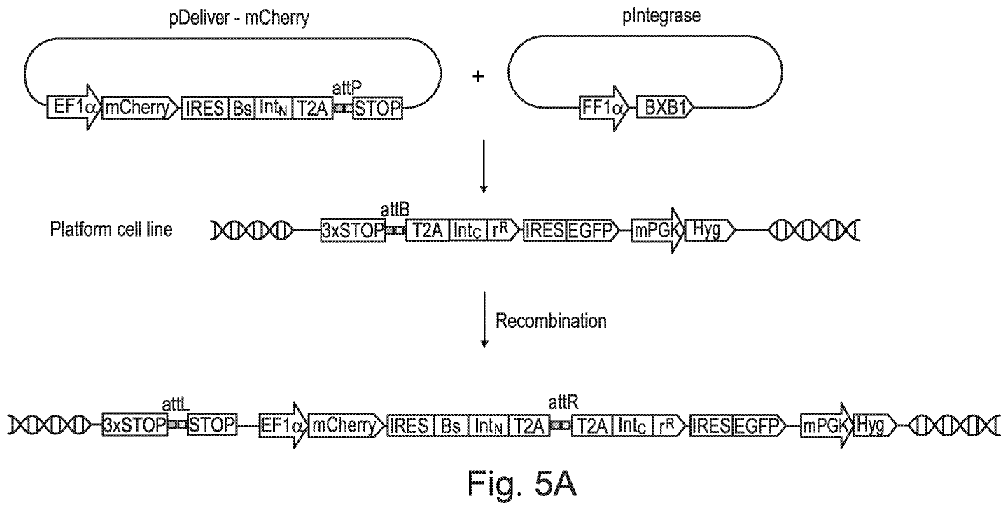

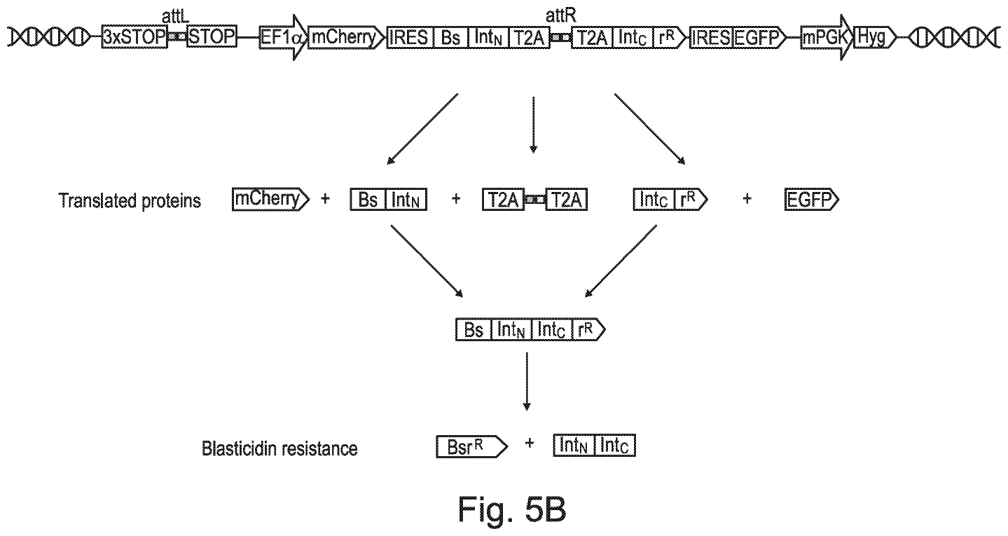

The system is expected to work in the same manner if the first split intein and split selectable marker combination comprises in the 5′ to 3′ direction an N-terminal part of the split selectable marker and a N-terminal part of the split intein and the second split intein and split selectable marker combination comprises in the 5′ to 3′ direction a C-terminal part of a split intein and a C-terminal part of a split selectable marker as detailed in FIGS. 4A and 4B.

### Example 3

The platform system described in Example 2 was further tested to assess its efficacy in retargeting cells to express other cargo proteins.

### Materials and Methods

### Cell Culture

CHO-K1 pPlatform cells which had been selected for stable integration of pPlatform ‘landing pad’ construct as described in Example 2 were cultured in RPMI 1640 (Gibco) with 10% FBS, 1× glutamate, 800 μg/ml hygromycin (Gibco) and 1× Pen/Strep. Hygromycin was included in the cell culture media to select for cells in which the pPlatform ‘landing pad’ construct.

### CD19 and FOLR1A Retargeting Constructs

Nucleotide sequences encoding CD19 or FOLR1A were integrated into the pDeliver retargeting plasmid in place of mCherry (the mCherry sequence which was replaced is underlined above in SEQ ID NO: 14).

The delivery nucleic acid pDeliver-CD19 comprises in 5′ to 3′ direction: an abscisic acid inducible promoter operably linked to N-terminal part of the split blasticidin selectable marker (Bs) attached to N terminal Npu DnaE split intein (Int_{N}), furin-T2A linker and attP site-specific recombination site. The construct further comprises a promoter operably linked to CD19, the cargo of the pDeliver-CD19 plasmid.

The delivery nucleic acid pDeliver-FOLR1A comprises in 5′ to 3′ direction: an abscisic acid inducible promoter operably linked to N-terminal part of the split blasticidin selectable marker (Bs) attached to N terminal Npu DnaE split intein (Int_{N}), furin-T2A linker and attP site-specific recombination site. The construct further comprises a promoter operably linked to FOLR1A, the cargo of the pDeliver-FOLR1A plasmid.

Retargeting pPlatform Cells with CD19 and FOLR1A Retargeting Constructs and BXB1 Expression Vector

CHO-K1 cells were seeded in 6-well plates and incubated for a period of 24 hrs before being transfected with the pDeliver retargeting plasmid (FOLR1A or CD19) and the pIntegrase BXB1 expression plasmid at 1:4 ratio, 2.5 μg total DNA, using Lipofectamine LTX transfection reagent. Following a 48-hour incubation period, media containing blasticidin (Gibco) was added to the cells and the cells cultured for a period of 3 weeks in selection medium. Following selection, expression of FOLR1A or CD19 was induced by the addition of abscisic acid and 24 hrs later the cells were analysed for the expression of EGFP, FOLR1A and CD19 via flow cytometry. Cells in which the pDeliver retargeting plasmid has been stably integrated in the correct position and orientation in the pPlatform ‘landing pad’ construct are expected to survive in the selection media due to the reconstruction of blasticidin resistance protein (as described in Example 2).

### Experimental Data

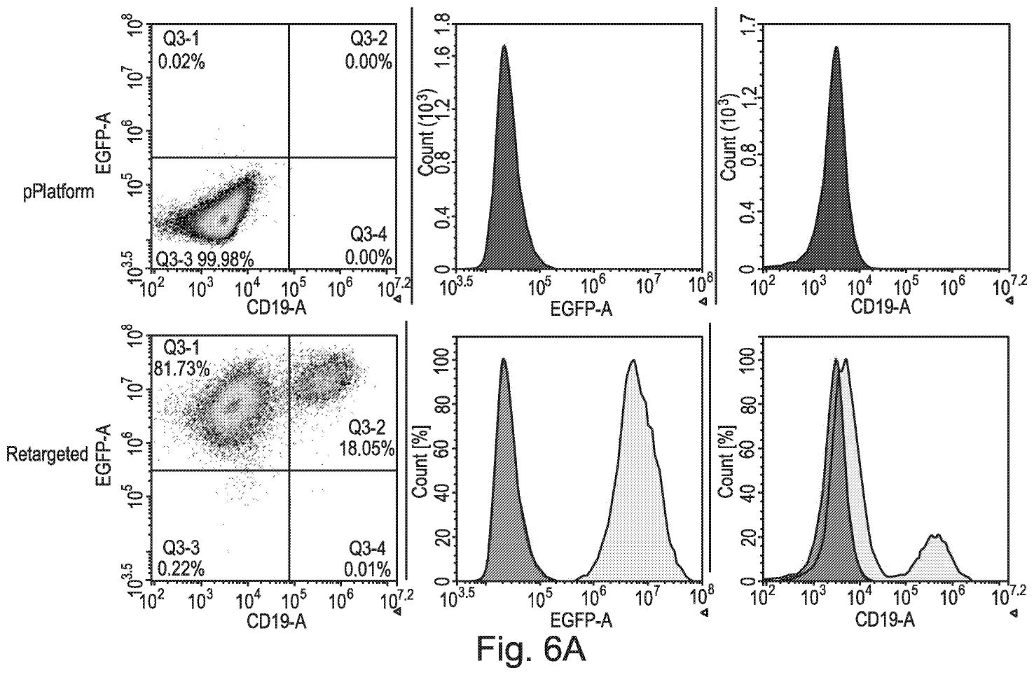

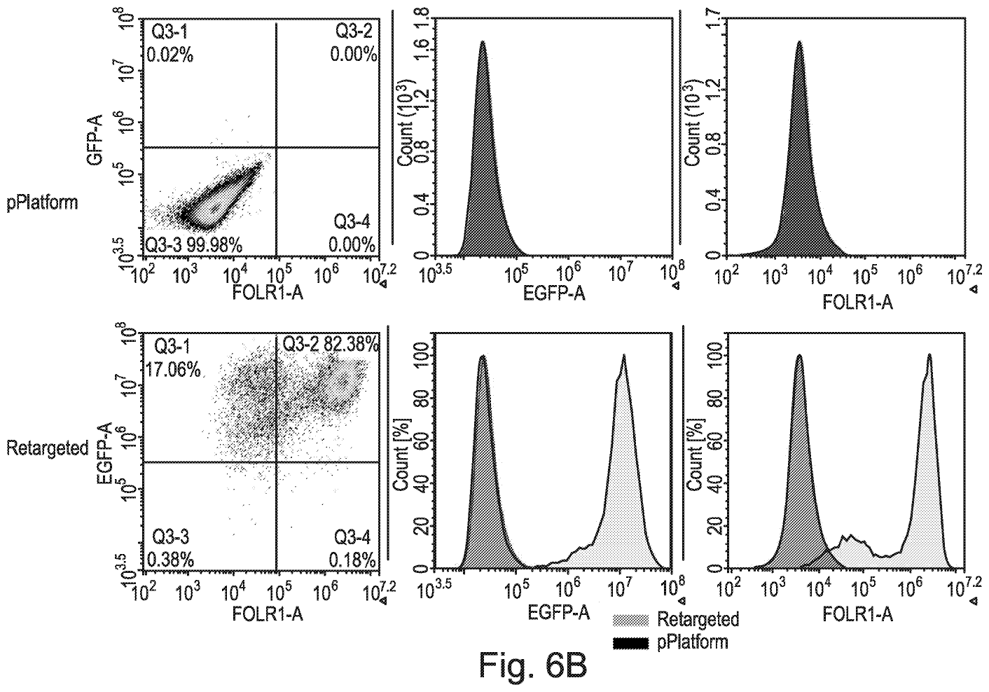

Following successful growth through both selection media described above, the retargeted cells were analysed by flow cytometry for expression of EGFP and CD19 or FOLR1A. The retargeted cells were compared to the parental platform pools which have undergone only media selection with hygromycin as described above i.e. the parental platform pools have only been selected for presence of the pPlatform “landing pad”. The results of this experiment are shown in FIG. 6A and FIG. 6B for pDeliver-CD19 and pDeliver-FOLR1A, respectively.

As described above, the pPlatform ‘landing pad’ construct comprises EGFP which is downstream of an internal ribosome entry site downstream of the C-terminal portion of the blasticidin gene (r^{R} in FIGS. 1A and 1B) and the NpuDnaE split intein C-terminal portion (Int_{C} in FIGS. 1A and 1B). EGFP is not expected to be expressed unless the construct in FIG. 1B has been created following successful integration of the pDeliver cargo sequence (CD19 or FOLR1A),

The pDeliver retargeting plasmids comprise CD19 or FOLR1A operably linked to a promoter so the cargo proteins are expected to be expressed provided that the pDeliver retargeting plasmid is present or integrated in the cell.

Cells which have been successfully retargeted are expected to express the cargo protein (CD19 of FOLR1A) and EGFP. FIGS. 6A and 6B show that the platform system can be successfully retargeted with different cargo proteins. The EGFP expression confirms that successful integration of the pDeliver retargeting plasmid has taken place at the platform site within the pPlatform ‘landing pad’ construct as EGFP is only expressed in the case of successful retargeting.

The expression profile of CD19 and FOLR1A show that both cargo proteins were successfully integrated into the respective cells (FIGS. 6A and 6B). The double peaks in both expression profiles suggest that there may be two integration sites (e.g. because the pPlatform “landing pad” has been inserted into two sites in the genome which are expressed to different levels) or a mixed population of cells (e.g. two cell populations in which the pPlatform “landing pad” has been inserted into different sites).

This experiment has successfully demonstrated that, as expected, the pPlatform and pDeliver constructs are useful tools for retargeting cell using a diverse range of cargo proteins of interest.

### CLAUSES

1. A system comprising:
a landing pad nucleic acid comprising in the 5′ to 3′ direction: an expression stop signal, a first site-specific recombination site, a first transcriptional or translational split mechanism, a C-terminal part of a split intein, and a C-terminal part of a split selectable marker;
a delivery nucleic acid comprising in the 5′ to 3′ direction: a second promoter operably linked to a N-terminal part of the split selectable marker, a N-terminal part of the split intein, a second transcriptional or translational split mechanism and a second site-specific recombination site, wherein the delivery nucleic acid further comprises a first promoter operably linked to a gene of interest; and
an integrating enzyme, or a nucleic acid encoding an integrating enzyme, wherein the integrating enzyme is configured to catalyse site specific recombination between the first site-specific recombination site and the second site-specific recombination site.

2. The system according to clause 1 wherein the integrating enzyme is a unidirectional serine integrase.

3. The system according to any preceding clause wherein the integrating enzyme is selected from the group consisting of: Bxb1, Wβ, BL3, R4, A118, TG1, MR11, ¢370, SPBc, TP901-1, φRV, FC1, K38, φBT1 and φC31, preferably selected from the group consisting of: Bxb1, φC31, R4 and φBT1.

4. The system according to any preceding clause wherein the integrating enzyme is Bxb1.

5. The system according to clause 4 wherein the nucleic acid encoding the integrating enzyme comprises or consists of SEQ ID NO: 13 or a functional variant thereof.

6. The system according to clause 1 and 2, wherein the first site-specific recombination site is selected from the group consisting of: Bxb1 attB, Wβ attB, BL3 attB, R4 attB, A118 attB, TG1 attB, MR11 attB, φC370 attB, SPBc attB, TP901 attB, RV attB, FC1 attB, φK38 attB, φBT1 attB and C31 attB.

7. The system according to clauses 1, 2 or 6, wherein the second site-specific recombination site is selected from the group consisting of: Bxb attP, Wβ attP, BL3 attP, R4 attP, A118 attP, TG1 attP, MR11 attP, φC370 attP, SPBc attP, TP901 attP, RV attP, FC1 attP, φK38 attP, φBT1 attP and C31 attP.

8. The system according to any preceding clause wherein the first site-specific recombination site is Bxb attB.

9. The system according to clause 8 wherein the first site-specific recombination site comprises or consists of SEQ ID NO: 1 or a functional variant thereof.

10. The system according to any preceding clause wherein the second site-specific recombination site is Bxb attP.

11. The system according to clause 10 wherein the second site-specific recombination site comprises or consists of SEQ ID NO: 2 or a functional variant thereof.

12. The system according to any preceding clause wherein the C-terminal part of the split intein is the C-terminal part of NpuDnaE, SspDnaB or SspDnaE intein.

13. The system according to any preceding clause wherein the N-terminal part of a split intein is the N-terminal part of NpuDnaE, SspDnaB or SspDnaE intein.

14. The system according to any preceding clause wherein the C-terminal part of the split intein is the C-terminal part of the NpuDnaE intein.

15. The system according to any preceding clause wherein the C-terminal part of the split intein comprises or consists of SEQ ID NO: 4 or a functional variant thereof.

16. The system according to any preceding clause wherein the N-terminal part of the split intein is the N-terminal part of the NpuDnaE intein.

17. The system according to any preceding clause wherein the N-terminal part of the split intein comprises or consists of SEQ ID NO: 5 or a functional variant thereof.

18. The system according to any preceding clause wherein the first transcriptional or translational split mechanism is internal ribosome entry site (IRES) or a 2A peptide.

19. The system according to any preceding clause wherein the first transcriptional or translational split mechanism is a 2A peptide selected from the group consisting of: F2A, P2A, E2A, T2A, GF2A, GP2A, GE2A and GT2A.

20. The system according to clause 19 wherein the first transcriptional or translational split mechanism is a 2A peptide with a furin recognition site.

21. The system according to any preceding clause wherein the first transcriptional or translational split mechanism is a T2A peptide.

22. The system according to any preceding clause wherein the first transcriptional or translational split mechanism is a T2A peptide with a furin recognition site.

23. The system according to any preceding clause wherein the first transcriptional or translational split mechanism comprises or consist of SEQ ID NO: 3 or SEQ ID NO: 22 or a functional variant thereof.

24. The system according to any preceding clause wherein the second transcriptional or translational split mechanism is internal ribosome entry site (IRES) or 2A peptide.

25. The system according to any preceding clause wherein the second transcriptional or translational split mechanism is a 2A peptide selected from the group consisting of: F2A, P2A, E2A, T2A, GF2A, GP2A, GE2A and GT2A.

26. The system according to clause 19 wherein the second transcriptional or translational split mechanism is a 2A peptide with a furin recognition site.

27. The system according to any preceding clause wherein the second transcriptional or translational split mechanism is a T2A peptide.

28. The system according to any preceding clause wherein the second transcriptional or translational split mechanism is a T2A peptide with a furin recognition site.

29. The system according to any preceding clause wherein the second transcriptional or translational split mechanism comprises or consist of SEQ ID NO: 3 or SEQ ID NO: 23 or a functional variant thereof.

30. The system according to any preceding clause wherein the expression stop signal is one or more stop codons, suitably 2, 3, 4, 5, 6, 7, 8, 9, or 10 stop codons.

31. The system according to any preceding clause wherein the expression stop signal is two sets of 3 stop codons.

32. The system according to any preceding clause wherein the expression stop signal comprises or consists of SEQ ID NO: 19 or a functional variant thereof or SEQ ID NO: 20 or a functional variant thereof.

33. The system according to any preceding clause wherein the split selectable marker is an antibiotic resistance gene, a gene encoding a fluorescent protein, a gene encoding glutamine synthetase or a gene encoding a luminescent protein.

34. The system according to any preceding clause wherein the C-terminal part of the split selectable marker and the N-terminal part of the split selectable marker are the C-terminal part and the N-terminal part of:
a hygromycin resistance gene (HygroR) split at amino acid position 52, 69, 89, 131, 171, 200, 240 or 292;
a puromycin resistance gene (PuroR) split at amino acid position 32, 84, 100 or 119;
a neomycin resistance gene (Neo^{R}) split at amino acid position 133 or 195;
a blasticidin resistance gene (BsrR) split at amino acid position 102;
a gene encoding the mScarlet fluorescent protein split at amino acid position 46, 48, 51, 75, 122, 140 or 163; or
a gene encoding the luciferase protein split at amino acid position 437.

35. The system according to any preceding clause wherein the C-terminal part of the split selectable marker and the N-terminal part of the split selectable marker are the C-terminal part and the N-terminal part of a blasticidin resistance gene (BsrR) split at amino acid position 102.

36. The system according to any preceding clause wherein the C-terminal part of the split selectable marker comprises or consist of SEQ ID NO: 6 or a functional variant thereof and the N-terminal part of a split selectable marker comprises or consist of SEQ ID NO: 7 or a functional variant thereof.

37. The system according to any preceding clause wherein the landing pad nucleic acid further comprises a third promoter operably linked to a second selectable marker.

38. The system according to any preceding clause wherein the second selectable marker is an antibiotic resistance gene, a gene encoding a fluorescent protein, or a gene encoding a luminescent protein, preferably an antibiotic resistance gene.

39. The system according to any preceding clause wherein the second selectable marker is selected from the group consisting of: kanamycin resistance gene, spectinomycin resistance gene, streptomycin resistance gene, ampicillin resistance gene, carbenicillin resistance gene, bleomycin resistance gene, erythromycin resistance gene, polymyxin B resistance gene, tetracycline resistance gene, chloramphenicol resistance gene, hygromycin resistance gene, puromycin resistance gene, neomycin resistance gene and blasticidin resistance gene.

40. The system according to any preceding clause wherein the second selectable marker is hygromycin.

41. The system according to any preceding clause wherein the landing pad nucleic acid further comprises an IREs followed by a further selectable marker immediately 3′ from the C-terminal part of a split selectable marker.

42. The system according to clause 41 wherein the further selectable marker is an antibiotic resistance gene, a gene encoding a fluorescent protein, or a gene encoding a luminescent protein.

43. The system according to any one of clauses 41-42 wherein the further selectable marker is a gene encoding a fluorescent protein.

44. The system according to any one of clauses 41-43 wherein the further selectable marker is selected from the group consisting of: EBFP, ECFP, EGFP, YFP, mHoneydew, mBanana, mOrange, tdTomato, mTangerine, mStrawberry, mCherry,mGrape1, mRaspberry, mGrape2 and mPlum.

45. The system according to any one of clauses 41-44 wherein the further selectable marker is EGFP.

46. A cell comprising the system according to any preceding clause.

47. The cell according to clause 46 wherein the cell is a HEK 293 cell or a CHO-K1 cell.

48. A cell comprising a landing pad nucleic acid, wherein the landing pad nucleic acid comprises in the 5′ to 3′ direction: an expression stop signal, a first site-specific recombination site, a first transcriptional or translational split mechanism, a C-terminal part of a split intein, and a C-terminal part of a split selectable marker.

49. The cell according to clause 48 wherein the landing pad nucleic acid is stably integrated into the genome of the cell.

50. The cell according to any one of clauses 48-49 further comprising:
a delivery nucleic acid comprising in the 5′ to 3′ direction: a second promoter operably linked to a N-terminal part of the split selectable marker, a N-terminal part of the split intein, a second transcriptional or translational split mechanism and a second site-specific recombination site, wherein the delivery nucleic acid further comprises a first promoter operably linked to the gene of interest; and
an integrating enzyme, or a nucleic acid encoding an integrating enzyme, wherein the integrating enzyme is configured to catalyse site specific recombination between the first site-specific recombination site and the second site-specific recombination site.

51. A method for providing a gene of interest and selecting a cell with a gene of interest, the method comprising the following steps:
providing a cell with a landing pad nucleic acid comprising in the 5′ to 3′ direction: an expression stop signal, a first site-specific recombination site, a first transcriptional or translational split mechanism, a C-terminal part of a split intein, and a C-terminal part of a split selectable marker;
providing the cell with (i) a delivery nucleic acid comprising in the 5′ to 3′ direction: a second promoter operably linked to a N-terminal part of the split selectable marker, a N-terminal part of the split intein, a second transcriptional or translational split mechanism and a second site-specific recombination site, wherein the delivery nucleic acid further comprises a first promoter operably linked to the gene of interest and (ii) an integrating enzyme, or a nucleic acid encoding an integrating enzyme, wherein the integrating enzyme is configured to catalyse site specific recombination between the first site-specific recombination site and the second site-specific recombination site;
selecting a cell which expresses the selectable marker gene, preferably wherein the steps are performed in the specified order.

52. The method according to clause 51, wherein the landing pad nucleic acid is provided via electroporation, microinjection, gene gun, impalefection, hydrostatic pressure, continuous infusion, sonication, calcium phosphate-based transfection, cationic polymers-based transfection, lipofection-based transfection, fugene-based transfection or viral delivery.

53. The method according to any one of clauses 51-52, wherein the landing pad nucleic acid is provided via lentiviral delivery.

54. The method according to any one of clauses 51-53, wherein the delivery nucleic acid is provided via electroporation, microinjection, gene gun, impalefection, hydrostatic pressure, continuous infusion, sonication, calcium phosphate-based transfection, cationic polymers-based transfection, lipofection-based transfection, fugene-based transfection or viral delivery.

55. The method according to any one of clauses 51-54, wherein the delivery nucleic acid is provided via lipofection-based transfection.

56. The method according to any one of clauses 51-55 further comprising culturing the cell under conditions to express the integrating enzyme, the C-terminal part of a split intein and the C-terminal part of a split selectable marker and the N-terminal part of the split selectable marker and a N-terminal part of the split intein.

57. A landing pad nucleic acid comprising in the 5′ to 3′ direction: an expression stop signal, a site-specific recombination site, a transcriptional or translational split mechanism, a part of a split intein, and a part of a split selectable marker.

58. A delivery nucleic acid comprising in the 5′ to 3′ direction: a second promoter operably linked to a part of a split selectable marker, a part of a split intein, a transcriptional or translational split mechanism and a site-specific recombination site, wherein the delivery nucleic acid further comprises a first promoter operably linked to a gene of interest.

59. A kit for providing and selecting a cell with a gene of interest, the kit comprising:
a landing pad nucleic acid comprising in the 5′ to 3′ direction: an the expression stop signal, a first site-specific recombination site, a first transcriptional or translational split mechanism, a C-terminal part of a split intein, and a C-terminal part of a split selectable marker;
a delivery nucleic acid comprising in the 5′ to 3′ direction: a second promoter operably linked to a N-terminal part of the split selectable marker, a N-terminal part of the split intein, a second transcriptional or translational split mechanism and a second site-specific recombination site, wherein the delivery nucleic acid further comprises a first promoter operably linked to a gene of interest; and
an integrating enzyme, or a nucleic acid encoding an integrating enzyme, wherein the integrating enzyme is configured to catalyse site specific recombination between the first site-specific recombination site and the second site-specific recombination site.

60. A kit for providing and selecting a cell with a gene of interest, the kit comprising:
a landing pad nucleic acid comprising in the 5′ to 3′ direction: an expression stop signal, a first site-specific recombination site, a first transcriptional or translational split mechanism, a first split intein and split selectable marker combination;
a delivery nucleic acid comprising in the 5′ to 3′ direction: a second split intein and split selectable marker combination N-terminal part of the split selectable marker, a N-terminal part of the split intein, a second transcriptional or translational split mechanism and a second site-specific recombination site, wherein the delivery nucleic acid further comprises a first promoter operably linked to a gene of interest; and
an integrating enzyme, or a nucleic acid encoding an integrating enzyme, wherein the integrating enzyme is configured to catalyse site specific recombination between the first site-specific recombination site and the second site-specific recombination site, optionally wherein the second split intein and split selectable marker combination is operably linked to a second promoter and wherein the first split intein and split selectable marker combination comprises in the 5′ to 3′ C-terminal part of a split intein and a C-terminal part of a split selectable marker and wherein the second split intein and split selectable marker combination comprises in the 5′ to 3′ direction N-terminal part of the split selectable marker and a N-terminal part of the split intein.

1. A system comprising:
a landing pad nucleic acid comprising in the 5′ to 3′ direction: an expression stop signal, a first site-specific recombination site, a first transcriptional or translational split mechanism, a first split intein and split selectable marker combination;
a delivery nucleic acid comprising in the 5′ to 3′ direction: a second split intein and split selectable marker combination, a second transcriptional or translational split mechanism and a second site-specific recombination site, wherein the delivery nucleic acid further comprises a first promoter operably linked to a gene of interest and
an integrating enzyme, or a nucleic acid encoding an integrating enzyme, wherein the integrating enzyme is configured to catalyse site specific recombination between the first site-specific recombination site and the second site-specific recombination site.

2. The system according to claim 1, wherein the second split intein and split selectable marker combination is operably linked to a second promoter or wherein the second split intein and split selectable marker combination is operably linked to the first promoter and separated from the gene of interest via IRES.

3. The system according to any preceding claim, wherein the first split intein and split selectable marker combination comprises in the 5′ to 3′ direction a C-terminal part of a split intein and a C-terminal part of a split selectable marker and the second split intein and split selectable marker combination comprises in the 5′ to 3′ direction a N-terminal part of the split selectable marker and a N-terminal part of the split intein.

4. The system according to claim 3, wherein the C-terminal part of the split intein and the N-terminal part of the split intein are configured to recombine the C-terminal part of the split selectable marker and the N-terminal part of the split selectable marker into a functional selectable marker.

5. The system according to any preceding claim wherein the integrating enzyme is a unidirectional serine integrase.

6. The system according to any preceding claim wherein the integrating enzyme is selected from the group consisting of: Bxb1, Wβ, BL3, R4, A118, TG1, MR11, φ370, SPBc, TP901-1, φRV, FC1, K38, φBT1 and φC31, preferably selected from the group consisting of: Bxb1, φC31, R4 and φBT1.

7. The system according to any preceding claim wherein the integrating enzyme is Bxb1.

8. The system according to claim 7 wherein the nucleic acid encoding the integrating enzyme comprises or consists of SEQ ID NO: 13 or a functional variant thereof.

9. The system according to any one of any preceding claim, wherein the first site-specific recombination site is selected from the group consisting of:
Bxb1 attB, Wβ attB, BL3 attB, R4 attB, A118 attB, TG1 attB, MR11 attB, φC370 attB, SPBc attB, TP901 attB, RV attB, FC1 attB, φK38 attB, φBT1 attB and C31 attB.

10. The system according to any one of any preceding claim wherein the second site-specific recombination site is selected from the group consisting of:
Bxb attP, Wβ attP, BL3 attP, R4 attP, A118 attP, TG1 attP, MR11 attP, φC370 attP, SPBc attP, TP901 attP, RV attP, FC1 attP, φK38 attP, φBT1 attP and C31 attP.

11. The system according to any preceding claim wherein the first site-specific recombination site is Bxb attB and the second site-specific recombination site is Bxb attP, optionally wherein the Bxb attB comprises or consists of SEQ ID NO: 1 or a functional variant thereof and the Bxb attP comprises or consists of SEQ ID NO: 2 or a functional variant thereof.

12. The system according to any preceding claim wherein the C-terminal part of the split intein is the C-terminal part of NpuDnaE, SspDnaB or SspDnaE intein.

13. The system according to any preceding claim wherein the N-terminal part of a split intein is the N-terminal part of NpuDnaE, SspDnaB or SspDnaE intein.

14. The system according to any one of claims 2-13 wherein the C-terminal part of the split intein is the C-terminal part of the NpuDnaE intein, optionally wherein the C-terminal part of the split intein comprises or consists of SEQ ID NO: 4 or a functional variant thereof and/or wherein the N-terminal part of the split intein is the N-terminal part of the NpuDnaE intein, optionally wherein the N-terminal part of the split intein comprises or consists of SEQ ID NO: 5 or a functional variant thereof.

15. The system according to any preceding claim wherein the first transcriptional or translational split mechanism is internal ribosome entry site (IRES) or a 2A peptide.

16. The system according to any preceding claim wherein the first transcriptional or translational split mechanism is a 2A peptide selected from the group consisting of: F2A, P2A, E2A, T2A, GF2A, GP2A, GE2A and GT2A.

17. The system according to any preceding claim wherein the first transcriptional or translational split mechanism is a 2A peptide with a furin recognition site.

18. The system according to any preceding claim wherein the first transcriptional or translational split mechanism is a T2A peptide with a furin recognition site, optionally wherein the first transcriptional or translational split mechanism comprises or consist of SEQ ID NO: 3 or SEQ ID NO: 22 or a functional variant thereof.

19. The system according to any preceding claim wherein the second transcriptional or translational split mechanism is internal ribosome entry site (IRES) or 2A peptide.

20. The system according to any preceding claim wherein the second transcriptional or translational split mechanism is a 2A peptide selected from the group consisting of: F2A, P2A, E2A, T2A, GF2A, GP2A, GE2A and GT2A.

21. The system according to claim 20 wherein the second transcriptional or translational split mechanism is a 2A peptide with a furin recognition site.

22. The system according to any preceding claim wherein the second transcriptional or translational split mechanism is a T2A peptide.

23. The system according to any preceding claim wherein the second transcriptional or translational split mechanism is a T2A peptide with a furin recognition site, optionally wherein the second transcriptional or translational split mechanism comprises or consist of SEQ ID NO: 3 or SEQ ID NO: 23 or a functional variant thereof.

24. The system according to any preceding claim wherein the expression stop signal is one or more stop codons, suitably 2, 3, 4, 5, 6, 7, 8, 9, or 10 stop codons and/or suitably two sets of 3 stop codons, optionally wherein the expression stop signal comprises or consists of SEQ ID NO: 19 or a functional variant thereof or SEQ ID NO: 20 or a functional variant thereof.

25. The system according to any preceding claim wherein the split selectable marker is an antibiotic resistance gene, a gene encoding a fluorescent protein, a gene encoding glutamine synthetase or a gene encoding a luminescent protein.

26. The system according to any one of claims 2-25 wherein the C-terminal part of the split selectable marker and the N-terminal part of the split selectable marker are the C-terminal part and the N-terminal part of:
a hygromycin resistance gene (HygroR) split at amino acid position 52, 69, 89, 131, 171, 200, 240 or 292;
a puromycin resistance gene (PuroR) split at amino acid position 32, 84, 100 or 119;
a neomycin resistance gene (Neo^{R}) split at amino acid position 133 or 195; or
a blasticidin resistance gene (BsrR) split at amino acid position 102, optionally wherein the C-terminal part of BsrR comprises or consists of SEQ ID NO: 6 or a functional variant thereof and the N-terminal part of BsrR comprises or consists of SEQ ID NO: 7 or a functional variant thereof;
a gene encoding the mScarlet fluorescent protein split at amino acid position 46, 48, 51, 75, 122, 140 or 163; or
a gene encoding the luciferase protein split at amino acid position 437.

27. The system according to any preceding claim wherein the C-terminal part of the split selectable marker and the N-terminal part of the split selectable marker are the C-terminal part and the N-terminal part of a blasticidin resistance gene (BsrR) split at amino acid position 102, optionally wherein the C-terminal part of the split selectable marker comprises or consists of SEQ ID NO: 6 or a functional variant thereof and the N-terminal part of a split selectable marker comprises or consists of SEQ ID NO: 7 or a functional variant thereof.

28. The system according to any preceding claim wherein the landing pad nucleic acid further comprises a third promoter operably linked to a second selectable marker.

29. The system according to any preceding claim wherein the second selectable marker is an antibiotic resistance gene, a gene encoding a fluorescent protein, or a gene encoding a luminescent protein, preferably an antibiotic resistance gene.

30. The system according to any preceding claim wherein the second selectable marker is selected from the group consisting of: kanamycin resistance gene, spectinomycin resistance gene, streptomycin resistance gene, ampicilin resistance gene, carbenicillin resistance gene, bleomycin resistance gene, erythromycin resistance gene, polymyxin B resistance gene, tetracycline resistance gene, chloramphenicol resistance gene, hygromycin resistance gene, puromycin resistance gene, neomycin resistance gene and blasticidin resistance gene.

31. The system according to any preceding claim wherein the second selectable marker is hygromycin.

32. The system according to any preceding claim wherein the landing pad nucleic acid further comprises an IREs followed by a further selectable marker immediately 3′ from the C-terminal part of a split selectable marker.

33. The system according to claim 32 wherein the further selectable marker is an antibiotic resistance gene, a gene encoding a fluorescent protein, or a gene encoding a luminescent protein.

34. The system according to any one of claims 32-33 wherein the further selectable marker is a gene encoding a fluorescent protein.

35. The system according to any one of claims 32-34 wherein the further selectable marker is selected from the group consisting of: EBFP, ECFP, EGFP, YFP, mHoneydew, mBanana, mOrange, tdTomato, mTangerine, mStrawberry, mCherry, mGrape1, mRaspberry, mGrape2 and mPlum.

36. The system according to any one of claims 32-35 wherein the further selectable marker is EGFP.

37. A cell comprising the system according to any preceding claim.

38. The cell according to claim 37 wherein the cell is a HEK 293 cell or a CHO-K1 cell.

39. A cell comprising a landing pad nucleic acid, wherein the landing pad nucleic acid comprises in the 5′ to 3′ direction: an expression stop signal, a first site-specific recombination site, a first transcriptional or translational split mechanism, a first split intein and split selectable marker combination, optionally wherein the landing pad nucleic acid is stably integrated into the genome of the cell, optionally wherein the first split intein and split selectable marker combination comprises in the 5′ to 3′ direction C-terminal part of a split intein and a C-terminal part of a split selectable marker, optionally wherein the cell further comprises:
a delivery nucleic acid comprising in the 5′ to 3′ direction: a second split intein and split selectable marker combination, a second transcriptional or translational split mechanism and a second site-specific recombination site, wherein the delivery nucleic acid further comprises a first promoter operably linked to the gene of interest; and
an integrating enzyme, or a nucleic acid encoding an integrating enzyme, wherein the integrating enzyme is configured to catalyse site specific recombination between the first site-specific recombination site and the second site-specific recombination site, optionally wherein the second split intein and split selectable marker combination is operably linked a second promoter and wherein the second split intein and split selectable marker combination comprises in the 5′ to 3′ direction N-terminal part of the split selectable marker and a N-terminal part of the split intein.

40. A method for providing a gene of interest and selecting a cell with a gene of interest, the method comprising the following steps:
providing a cell with a landing pad nucleic acid comprising in the 5′ to 3′ direction: an expression stop signal, a first site-specific recombination site, a first transcriptional or translational split mechanism, a first split intein and split selectable marker combination;
providing the cell with (i) a delivery nucleic acid comprising in the 5′ to 3′ direction: a second split intein and split selectable marker combination, a second transcriptional or translational split mechanism and a second site-specific recombination site, wherein the delivery nucleic acid further comprises a first promoter operably linked to the gene of interest and (ii) an integrating enzyme, or a nucleic acid encoding an integrating enzyme, wherein the integrating enzyme is configured to catalyse site specific recombination between the first site-specific recombination site and the second site-specific recombination site;
selecting a cell which expresses the selectable marker gene, preferably wherein the steps are performed in the specified order, optionally wherein the second split intein and split selectable marker combination is operably linked a second promoter and wherein the first split intein and split selectable marker combination comprises in the 5′ to 3′ direction C-terminal part of a split intein and a C-terminal part of a split selectable marker and wherein the second split intein and split selectable marker combination comprises in the 5′ to 3′ direction N-terminal part of the split selectable marker and a N-terminal part of the split intein.

41. The method according to claim 40, wherein the landing pad nucleic acid is provided via electroporation, microinjection, gene gun, impalefection, hydrostatic pressure, continuous infusion, sonication, calcium phosphate-based transfection, cationic polymers-based transfection, lipofection-based transfection, fugene-based transfection or viral delivery.

42. The method according to claim 40 or 41 wherein the landing pad nucleic acid is provided via lentiviral delivery and/or wherein the delivery nucleic acid is provided via lipofection-based transfection.

43. The method according to any one of claims 40-42 further comprising culturing the cell under conditions to express the integrating enzyme, the C-terminal part of a split intein and the C-terminal part of a split selectable marker and the N-terminal part of the split selectable marker and a N-terminal part of the split intein.

44. The method according to any one of claims 40-43, wherein, following providing the cell with (i) the delivery nucleic acid and (ii) the integrating enzyme, or the nucleic acid encoding an integrating enzyme, the integrating enzyme catalyses integration of the delivery nucleic acid into the landing pad nucleic acid, and thereby the selectable marker is reconstructed following translation of the parts of the split selectable marker prior to selecting the cell which expresses the selectable marker gene.

45. The method according to any one of claims 40-44, wherein the split selectable marker is re-joined to form a functional selectable marker when the gene of interest is successfully integrated in the correct location.

46. The method according to any one of claims 40-45 wherein expression of the selectable marker gene indicates that the gene of interest has been integrated into the landing pad nucleic acid.

47. A landing pad nucleic acid comprising in the 5′ to 3′ direction: an expression stop signal, a site-specific recombination site, a transcriptional or translational split mechanism, a part of a split intein, and a part of a split selectable marker.

48. A delivery nucleic acid comprising in the 5′ to 3′ direction: a part of a split selectable marker, a part of a split intein, a transcriptional or translational split mechanism and a site-specific recombination site, wherein the delivery nucleic acid further comprises a first promoter operably linked to a gene of interest, optionally wherein the part of the split selectable marker and the part of the split intein is operably linked to a second promoter.

49. A kit for providing and selecting a cell with a gene of interest, the kit comprising:
a landing pad nucleic acid comprising in the 5′ to 3′ direction: an expression stop signal, a first site-specific recombination site, a first transcriptional or translational split mechanism, a first split intein and split selectable marker combination;
a delivery nucleic acid comprising in the 5′ to 3′ direction: a second split intein and split selectable marker combination, a second transcriptional or translational split mechanism and a second site-specific recombination site, wherein the delivery nucleic acid further comprises a first promoter operably linked to a gene of interest; and
an integrating enzyme, or a nucleic acid encoding an integrating enzyme, wherein the integrating enzyme is configured to catalyse site specific recombination between the first site-specific recombination site and the second site-specific recombination site, optionally wherein the second split intein and split selectable marker combination is operably linked to a second promoter and wherein the first split intein and split selectable marker combination comprises in the 5′ to 3′ direction a nucleotide sequence encoding a C-terminal part of a split intein and a C-terminal part of a split selectable marker and wherein the second split intein and split selectable marker combination comprises in the 5′ to 3′ direction a nucleotide sequence encoding a N-terminal part of the split selectable marker and a N-terminal part of the split intein.
